(19) United States
(12) Patent Application Publication
(10) Pub. No.: US 2024/0049597 A1
(43) Pub. Date: Feb. 8, 2024

(54) ORGANIC LUMINESCENT COMPOUND, AND ORGANIC ELECTROLUMINESCENT ELEMENT COMPRISING SAME

(71) Applicant: SOLUS ADVANCED MATERIALS CO., LTD, lksan-si, Jeollabuk-do (KR)

(72) Inventors: Jeongkeun PARK, Yongin-si, Gyeonggi-do (KR); Minsik EUM, Yongin-si, Gyeonggi-do (KR); Jaeyi SIM, Yongin-si, Gyeonggi-do (KR); Doshik KIM, Yongin-si, Gyeonggi-do (KR)

(73) Assignee: SOLUS ADVANCED MATERIALS CO., LTD, lksan-si, Jeollabuk-do (KR)

(21) Appl. No.: 18/266,370

(22) PCT Filed: Dec. 10, 2021
(86) PCT No.: PCT/KR2021/018731

§ 371 Date: Jun. 9, 2023

(30) Foreign Application Priority Data
Dec. 11, 2020 (KR) 10-2020-0173693

(51) Int. Cl. H10K 85/60; C07D 307/91; C09K 11/06

(52) U.S. Cl. CPC H10K 85/6574; C07D 307/91; C09K 11/06; H10K 85/626; H10K 50/11

(57) ABSTRACT

A novel organic compound and an organic electroluminescent element using the organic compound are disclosed. The novel organic compound has excellent thermal stability and light-emitting ability. Organic electroluminescent elements containing the organic compound in one or more organic layers has improved properties such as a high light-emitting efficiency, a low driving voltage, and a long lifespan.

### TECHNICAL FIELD

The present invention relates to a novel organic compound and an organic electroluminescent element using the same and, more particularly, to a compound having excellent thermal stability and light-emitting ability; and an organic electroluminescent element that contains the compound in one or more organic layers and thus has improved properties such as a light-emitting efficiency, a low driving voltage, and a long lifespan.

### BACKGROUND ART

Starting from Bernanose's observation of light emission from organic thin films in the 1950s, the study of organic electroluminescent (“EL”) elements led to blue electroluminescence using anthracene monocrystals in 1965, and Tang suggested in 1987 an organic EL element in a stack structure which may be divided into functional layers of hole layers and light emitting layers. Then, in order to develop high efficiency, long lifespan organic EL elements, organic layers each having distinctive characteristics have been introduced in the EL elements, leading to the development of specialized materials used therein.

In organic EL elements, upon application of voltage between two electrodes, holes are injected from an anode (e.g., positive electrode) to an organic layer and electrons are injected from a cathode (e.g., negative electrode) into the organic layer. Injected holes and electrons meet each other to form excitons, and light emission occurs when the excitons fall to a ground state. In such a case, materials used for the organic layer may be classified into, for example, luminescent (e.g., light emitting) materials, hole injection materials, hole transport materials, electron transport materials and electron injection materials depending on their function.

Light emitting materials of an organic EL element may be classified into blue-, green- and red-light emitting materials depending on their emission colors. Besides, yellow and orange light emitting materials may also be used as such a light emitting material for realizing better natural colors. In addition, a host/dopant system may be employed in the light emitting material to increase color purity and luminescence efficiency through energy transferring. Dopant materials may be classified into fluorescent dopants using organic materials and phosphorescent dopants using metal complex compounds which include heavy atoms such as Ir and Pt. The developed phosphorescent materials may improve the luminescence efficiency theoretically up to four times as compared to fluorescent materials, so attention is given to phosphorescent dopants as well as phosphorescent host materials. To date, NPB, BCP and Alq₃, for example, are widely known as materials used in the hole injection layer, the hole transporting layer, the hole blocking layer and the electron transporting layer, and anthracene derivatives have been reported as fluorescent dopant/host materials for light emitting materials. Particularly, metal complex compounds including Ir, such as FIrpic, Ir(ppy)₃, and Ir(btp)₂(acac), are known as phosphorescent dopant materials for efficiency improvement among light emitting materials, and they are used as blue, green and red dopant materials. Up to this day, CBP has shown excellent properties as a phosphorescent host material.

However, conventional materials, despite their good luminescence properties, have low glass transition temperatures and poor thermal stability and thus are not satisfactory in terms of lifespan characteristics of organic EL elements. Accordingly, there is a demand for the development of an organic layer material having excellent performance.

Meanwhile, deuterium has a natural abundance of approximately 0.015%. Deuterated compounds rich in concentrations of deuterium are well known. Deuterated aromatic compounds have been used to study chemical reactions and metabolic pathways and have also been used as raw materials for pharmaceuticals, agricultural chemicals, functional materials, and analytical tracers. Some deuterated electroluminescent materials have been reported to exhibit improved performance (efficiency, lifespan) compared to non-deuterated isotopomers (see, e.g., Tong, et al. J. Phys. Chem. C 2007, 111, 3490-4). Current methods of synthesizing deuterated compounds may require self-weighting to achieve high levels of deuteration. Since these methods are expensive or time-consuming, they are not suitable in terms of cost and efficiency. Accordingly, there is a continuous need for an improved manufacturing method for synthesizing deuterated aromatic compounds.

### DETAILED DESCRIPTION OF THE INVENTION

### Technical Objectives

The present invention has been conceived to solve the above problems, and specifically, is directed to a novel organic compound applicable to organic electroluminescent elements and having excellent characteristics such as thermal stability, luminescence, hole injection, hole transport, luminescence, electron transport, and electron injection, and more preferably to a blue fluorescent light emitting layer material excellent in thermal stability and luminescence.

The present invention is also directed to an organic electroluminescent element including the novel organic compound described above and thereby has thermal stability, a low driving voltage, a high luminescence efficiency, and an improved lifespan.

Other objectives and advantages of the present invention may be more clearly explained by the following detailed description and claims.

### Technical Means to Solve the Problem

To achieve the above objectives, the present invention provides a compound represented by the following Chemical Formula 1:

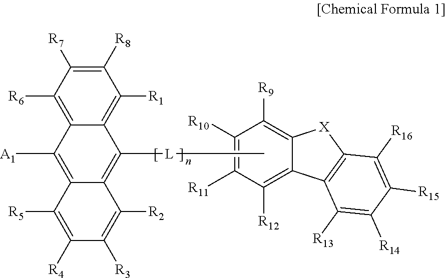

wherein in Chemical Formula 1,
R₁ to R₈ are the same as or different from each other, each independently being hydrogen or deuterium, provided that at least one of R₁ to R₈ is deuterium,
X is O, S or CRₐR_{b},
Rₐ and R_{b} are the same as or different from each other, each independently being a C₁ to C₄₀ alkyl group or a C₆ to C₆₀ aryl group, or Rₐ and R_{b} being bonded to each other to form a condensed ring,
R₉ to R₁₄ are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group,
L is a single bond or is selected from: a C₆ to C₁₈ arylene group and a heteroarylene group having 5 to 18 nuclear atoms,
n is an integer in a range from 0 to 2,
A₁ is a substituent represented by the following Chemical Formula 2,

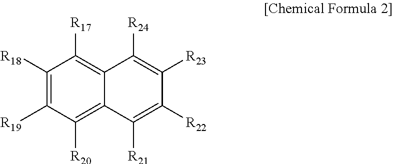

in Chemical Formula 2,
one of R₁₇ to R₂₄ is bonded to Chemical Formula 1, and another one of R₁₇ to R₂₄ is bonded to an aryl group A₂ having at least one deuterium,
Chemical Formula 1 and the aryl group A₂ having at least one deuterium are bonded in an ortho position,
the others of R₁₇ to R₂₄ that are not bonded to Chemical Formula 1 and the aryl group having at least one deuterium are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group, and
the arylene group and the heteroarylene group of L and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the phosphine group, the phosphine oxide group, and the arylamine group of R₉ to R₁₄, Rₐ, R_{b}, and the others of R₁₇ to R₂₄ that are not bonded to Chemical Formula 1 and the aryl group having at least one deuterium are each independently substitutable with one or more kinds of substituents selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and when the substituents are plural in number, the substituents are the same as or different from each other.

The present invention further provides an organic electroluminescent element including: an anode, a cathode, and one or more organic layers disposed between the anode and the cathode, wherein at least one of the one or more organic layers includes the compound represented by Chemical Formula 1.

In such a case, at least one of the organic layers including the compound represented by Chemical Formula 1 may be selected from: a hole injection layer, a hole transport layer, a light emitting auxiliary layer, a light emitting layer, an electron transport layer, and an electron injection layer, and may preferably be a light emitting layer. In such a case, the compound represented by Chemical Formula 1 may be used as a blue fluorescent host material of a light emitting layer.

### Effects of the Invention

According to an embodiment of the present invention, a compound represented by Chemical Formula 1 may be used as a material for an organic layer of an organic electroluminescent (“EL”) element due to its excellent thermal stability and luminescent properties.

In particular, when the compound represented by Chemical Formula 1 of the present invention is used as a fluorescent host material, organic EL elements having characteristics of low voltage, high efficiency, and long lifespan compared to conventional host materials may be manufactured, and full color display panels having improved performance and lifespan may further be manufactured.

Effects according to the present invention are not limited by the description exemplified above, and more diverse effects are included in the present specification.

### DETAILED DESCRIPTION OF THE INVENTION

Hereinafter, the present invention will be described in detail.

<Organic Compound>

The present invention is directed to a deuterated electroluminescent material having a novel structure capable of simultaneously implementing characteristics of an element such as a low voltage, a high efficiency, and a long lifespan.

According to the present invention, a compound represented by Chemical Formula 1 has a basic skeleton in which a deuterated anthracene moiety and a non-deuterated naphthalene (e.g., A₁) moiety are included as a core, another deuterated aryl group (e.g., A₂) is substituted at an ortho position of the naphthyl group (A₁), and a dibenzo-based moiety (e.g., X-containing ring) is bonded to one phenyl ring of the deuterated anthracene directly or through a separate linker (e.g., L).

Specifically, the compound of Chemical Formula 1 may be improved in terms of lifespan characteristics of an element because deuterium is substituted in the anthracene core, and characteristics such as voltage and efficiency of the element may be further improved because the naphthyl group A₁ having a high carrier mobility is introduced into the deuterated anthracene core. When the naphthyl group is substituted with deuterium, a bond dissociation energy increases according to the Marcus theory, so stability of the element is increased compared to a material containing a naphthyl group that is unsubstituted with deuterium, such that lifespan characteristics of the element may be further improved.

In particular, in the present invention, in introducing the deuterated aryl group A₂ into the naphthyl group A₁ to improve lifespan, two deuterated moieties, for example, a deuterated anthracene and a deuterated aryl group A₂, are bonded in an ortho-position with respect to the naphthyl group A₁ to impart a steric effect. In such a way, the introduction of the deuterated aryl group A₂ in an ortho bonding position may result in improved efficiency by preventing packing between molecules. In addition, blue fluorescence characteristics may be maximized by introducing the dibenzo-based moiety, particularly a dibenzofuran group, which is known as a blue fluorescence material.

In the present invention, since a plurality of deuterium is included in the basic skeletal structure described above, color purity may be further maximized compared to compounds of the same structure without deuterium, and an intramolecular bonding strength between carbon and hydrogen, which is weakened, may be further increased to significantly improve lifespan characteristics.

Furthermore, each of red and green light emitting layers of an organic electroluminescent (“EL”) element uses a phosphorescent material, and their technological maturity is currently high. On the other hand, in the case of a blue light emitting layer which may be classified into a fluorescent material and a phosphorescent material, the fluorescent material is in need of performance improvement, and the blue phosphorescent material is still under development and thus has a high entry barrier. That is, since the blue light emitting layer has a high development potential but has a relatively high technical difficulty, there is a limit to performance improvement (e.g., driving voltage, efficiency, lifespan, etc.) of a blue organic EL element including the blue light emitting layer. In this respect, since the compound of Chemical Formula 1 according to the present invention may be usefully applicable as a material for a blue fluorescent light emitting layer, it may simultaneously improve the performance of a blue light emitting layer as well as characteristics of an organic EL element including the blue light emitting layer such as a low voltage, a high efficiency and a long lifespan of the organic EL element.

Accordingly, the compound represented by Chemical Formula 1 may improve the luminescence characteristics, as well as characteristics such as electron injection/transport ability, luminescence efficiency, driving voltage, lifespan, and the like of an organic EL element. Accordingly, the compound of Chemical Formula 1 according to the present invention may be used as a material for any one of a hole injection layer, a hole transport layer, a light emitting layer, an electron transport layer, and an electron injection layer, which are organic layers of an organic EL element, and may preferably be a light emitting layer material (a blue fluorescent host material). In particular, when the compound represented by Chemical Formula 1 of the present invention is used as a blue fluorescent host material, an organic EL element having a low driving voltage, a high efficiency and a long lifespan compared to conventional light emitting host materials (e.g., CBP) may be manufactured, and furthermore, a full color display panel with improved characteristics such as a high efficiency and a long lifespan may be manufactured.

Specifically, the compound represented by Chemical Formula 1 according to the present invention may have a basic skeleton in which a deuterated anthracene moiety and a non-deuterated naphthalene (e.g., A₁) moiety are used as a core, another deuterated aryl group (e.g., A₂) is substituted at an ortho position of the naphthyl group A₁, and a dibenzo-based moiety (e.g., X-containing ring) is bonded to one phenyl ring of the deuterated anthracene directly or through a separate linker (e.g., L).

The anthracene contains at least one deuterium (D). In an embodiment of anthracene, R₁ to R₈ are the same as or different from each other, and each independently represent hydrogen or deuterium, provided that at least one of R₁ to R₈ is deuterium. Specifically, at least one of R₁ to R₈ may be deuterium and the others may be hydrogen, and more specifically, all of R₁ to R₈ may be deuterium. By including the plurality of deuterium in such a way, the lifespan characteristics of the element may be improved.

The non-deuterated naphthyl A₁ moiety having excellent carrier characteristics is bonded to the deuterated anthracene, and the deuterated aryl group A₂ is bonded to a specific position of the naphthyl A₁ group. Specifically, the deuterated anthracene and the deuterated aryl group A₂ are bonded to each other in an ortho position with respect to a carbon of the naphthyl group A₁. As such, the two deuterated moieties (anthracene and aryl group A₂) ortho-bonded with respect to the naphthyl group A₁ may enhance both voltage and efficiency characteristics of an element through steric hindrance effects and intermolecular packing prevention effects.

The naphthyl group A₁ may be represented by the following Chemical Formula 2.

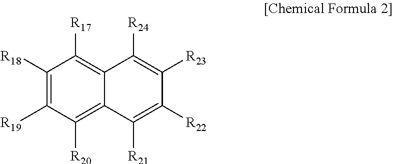

Among R₁₇ to R₂₄ in Chemical Formula 2, the two bonding positions where bonds with Chemical Formula 1 and the deuterated aryl group A₂ are made are not particularly limited as long as they are in an ortho position. For example, any one of R₁₇ to R₂₄ is bonded to Chemical Formula 1, and any one of the others is bonded to the aryl group A₂ having at least one deuterium, where Chemical Formula 1 and the aryl group A₂ having deuterium are bonded in the ortho position. Specifically, one of R₁₇ and R₁₈ in Chemical Formula 2 may be bonded to Chemical Formula 1, and the other may be bonded to the aryl group A₂ having at least one deuterium. That is, in the substituent represented by Chemical Formula 2, one of R₁₇ and R₁₈ is bonded to Chemical Formula 1, and the other of R₁₇ and R₁₈ is bonded to the aryl group A₂ having deuterium.

In addition, the others of R₁₇ to R₂₄ that are not bonded to each of Chemical Formula 1 and the aryl group A₂ having at least one deuterium may be the same as or different from each other, and may each independently be selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group. Specifically, R₉ to R₁₄ that are not bonded to each of Chemical Formula 1 and the aryl group A₂ having deuterium may be the same as or different from each other, and may each independently be selected from: hydrogen, deuterium, a C₁ to C₄₀ alkyl group and a C₆ to C₆₀ aryl group.

The deuterated aryl group A₂ introduced into the naphthyl group A₁ of Chemical Formula 2 is not particularly limited as long as it is a conventional C₆ to C₆₀ aryl group known in the art in which at least one deuterium (D) is substituted. For example, it may be a C₆ to C₁₂ aryl group such as phenyl group or a naphthyl group.

For a specific example, the aryl group A₂ having deuterium may be selected from structural formulas represented by the following Chemical Formulas 3a to 3c. However, it is not particularly limited thereto.

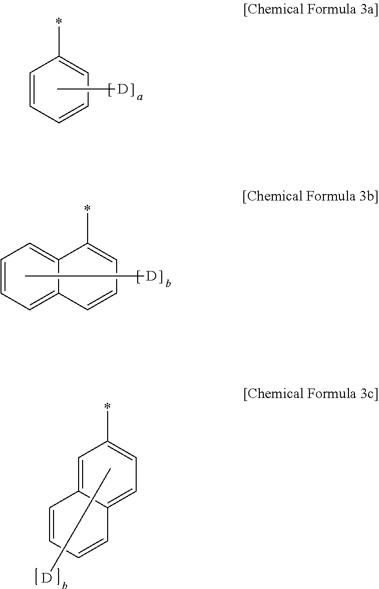

In Chemical Formulas 3a to 3c,
* indicates a site where a bond to Chemical Formula 2 is made, and
a is an integer in a range from 0 to 5, and b is an integer in a range from 0 to 7.

Such an aryl group A₂ having deuterium may be embodied as shown in the following structural formulas. However, it is not limited thereto.

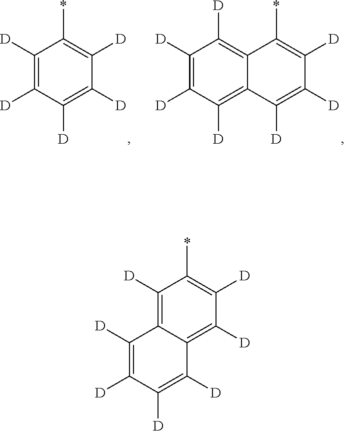

A specific example of a moiety of Chemical Formula 2 (naphthyl group, A₁) into which the aryl group A₂ having deuterium is introduced according to an embodiment of the present invention may be represented by the following structural formulas.

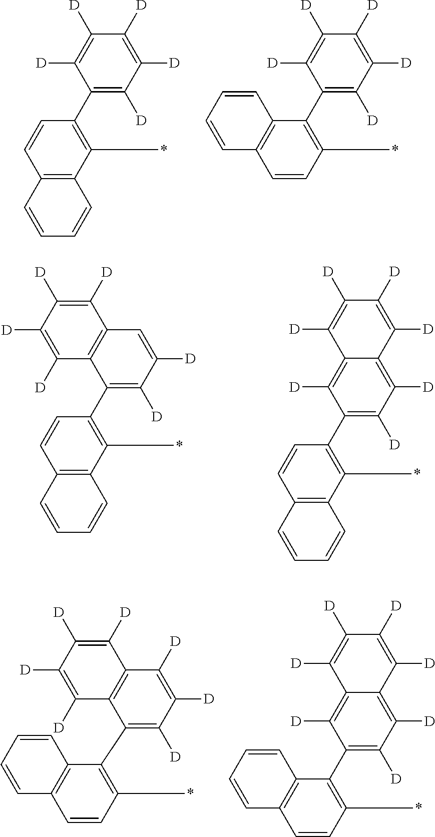

In such a case, although not shown in the above structural formulas, at least one substituent known in the art (e.g., the same as the definition of R₉) may be substituted.

In the deuterated anthracene according to the present invention, a dibenzo-based moiety (e.g., X-containing ring) is introduced through direct bonding or through a separate linker (e.g., L). The dibenzo-based moiety (e.g., X-containing ring) is superior in terms of a high glass transition temperature (Tg) and thermal stability. In an embodiment of such a dibenzo-based moiety (e.g., X-containing ring), Y is O, S, or CRₐR_{b}, specifically O (dibenzofuran) or S (dibenzothiophene), and preferably is O.

In such a case, Rₐ and R_{b} may be the same as or different from each other and may each independently be a C₁ to C₄₀ alkyl group or a C₆ to C_{H} aryl group, or they (e.g., Rₐ and R_{b}) may be bonded to each other to form a condensed (e.g., fused) ring (e.g., a spiro ring).

In an embodiment, R₉ to R₁₆ may be substitutable as various substituents in the dibenzo-based moiety. These R₉ to R₁₆ may be the same as or different from each other, and may each independently be selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group, or adjacent groups of R₉ to R₁₆ (e.g., among R₁₃ to R₁₆) may be bonded to each other to form a condensed ring. Specifically, R₉ to R₁₆ may be the same as or different from each other, and may each independently be selected from: hydrogen, deuterium, a C₁ to C₄₀ alkyl group and a C₆ to C₆₀ aryl group. In such a case, at least one of R₉ to R₁₆, specifically at least one of R₁₃ to R₁₆ may be a C₆ to C₆₀ aryl group, or a C₆ to C₆₀ aryl group substituted with at least one deuterium.

The aforementioned dibenzo-based moiety (e.g., X-containing ring) is bonded to the deuterated anthracene directly or through a linker L. As such, when a separate linker L is present between the dibenzo-based moiety and the deuterated anthracene, a HOMO region may be expanded to give a benefit to a HOMO-LUMO distribution, and charge transfer efficiency may be increased through an appropriate overlap of HOMO-LUMO.

This linker L is not particularly limited, and may be a single bond or a common divalent group linker known in the art. Specifically, L may be the same as or different from each other, and may each independently be a single bond (e.g., direct bond) or selected from: a C₆ to C₁₈ arylene group and a heteroarylene group having 5 to 18 nuclear atoms. Specific examples of the arylene group and the heteroarylene group may include, for example, a phenylene group, a biphenylene group, a pyrrolylene group, an imidazolylene group, an oxazolylene group, a thiazolylene group, a triazolylene group, a pyridinylene group, and a pyrimidinylene group. More specifically, L may be the same as or different from each other, and may each independently be a single bond or selected from: a C₆ to C₁₂ arylene group and a heteroarylene group having 5 to 12 nuclear atoms.

In such a case, the number (e.g., n) of linkers may each be an integer in a range from 0 to 2. For example, when n is 0, L may be a single bond. In addition, when n is greater than 0 and less than or equal to 2, L may be a substituent other than a single bond in the above definition of the linker.

For a specific example, L may be a single bond or a linking group selected from the following structural formulas.

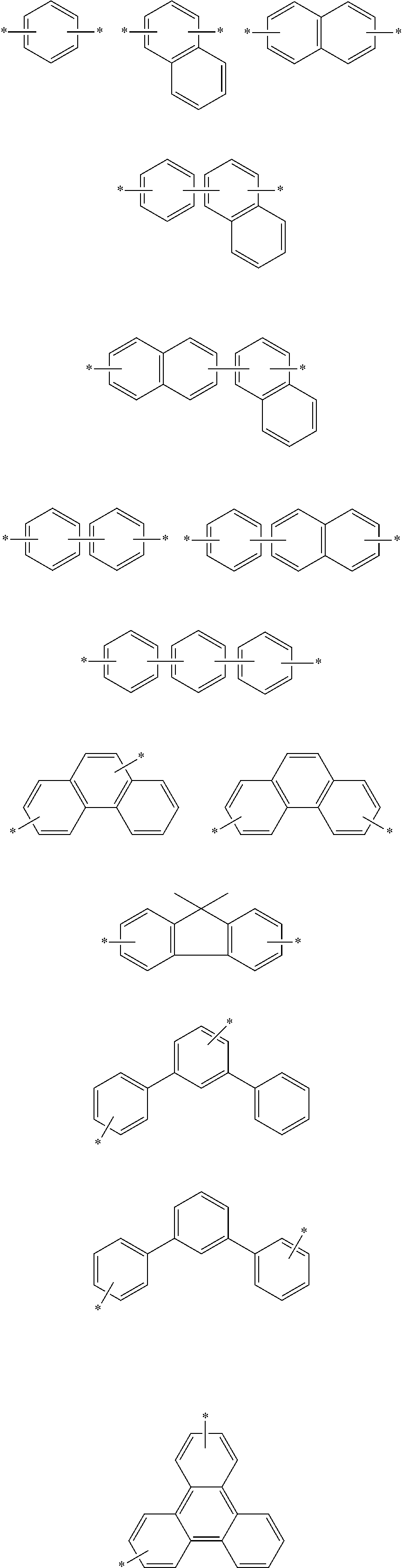

In the above structural formulas,
* indicates a site where a bond with Chemical Formula 1 is made. In addition, although not shown in the above structural formulas, at least one substituent known in the art (e.g., the same as the definition of R₉) may be substituted.

In the aforementioned Chemical Formula 1, the arylene group and the heteroarylene group of L and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the phosphine group, the phosphine oxide group, and the arylamine group of R₉ to R₁₄, Rₐ, R_{b}, and the others of R₁₇ to R₂₄ that are not bonded to each of Chemical Formula 1 and the aryl group having at least one deuterium may each independently be substitutable with one or more kinds of substituents selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and when the substituents are plural in number, the substituents may be the same as or different from each other.

In an embodiment of the present invention, the compound represented by Chemical Formula 1 may be represented by any one of Chemical Formulas 4 to 9 according to the type of the naphthyl group A₁ and the deuterated aryl group A₂ in Chemical Formula 2.

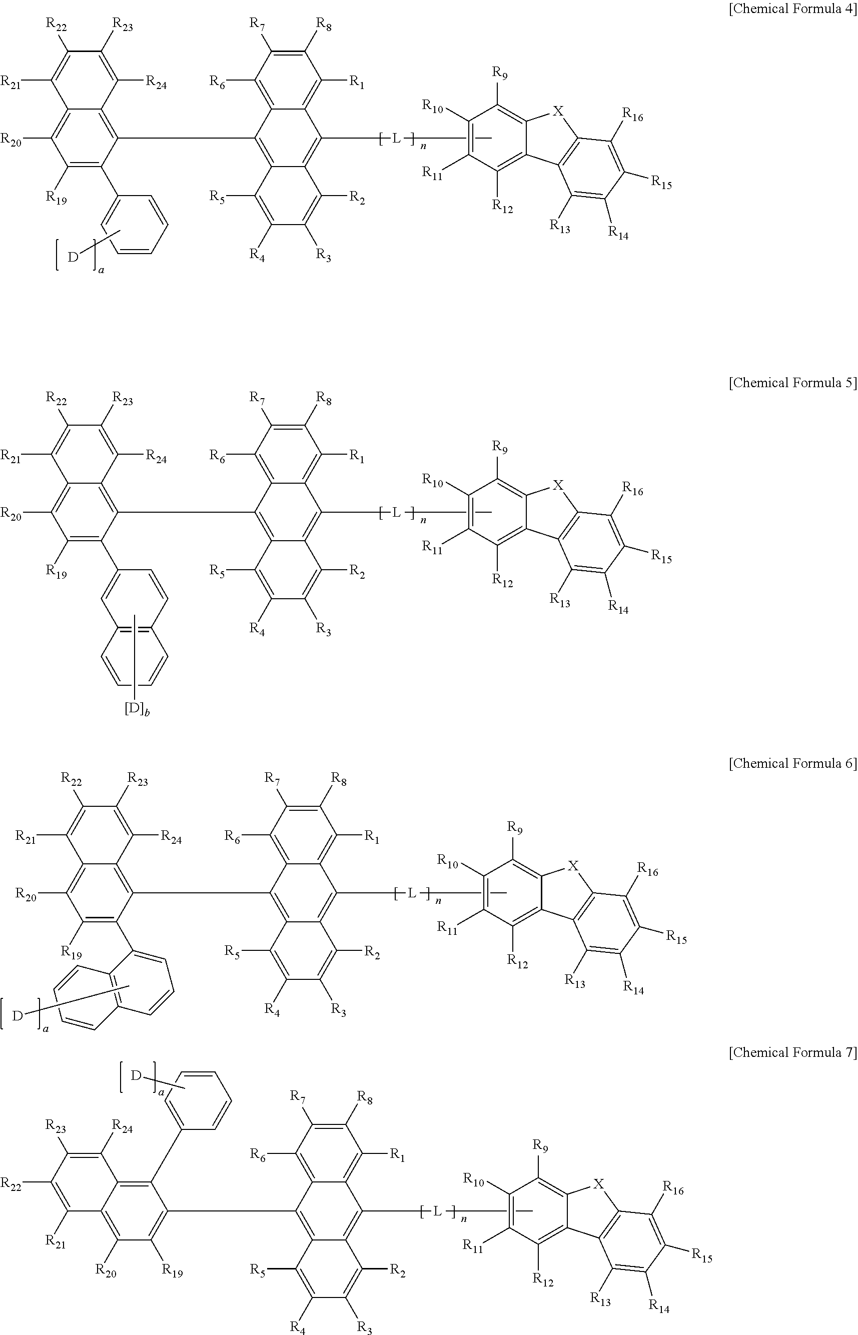

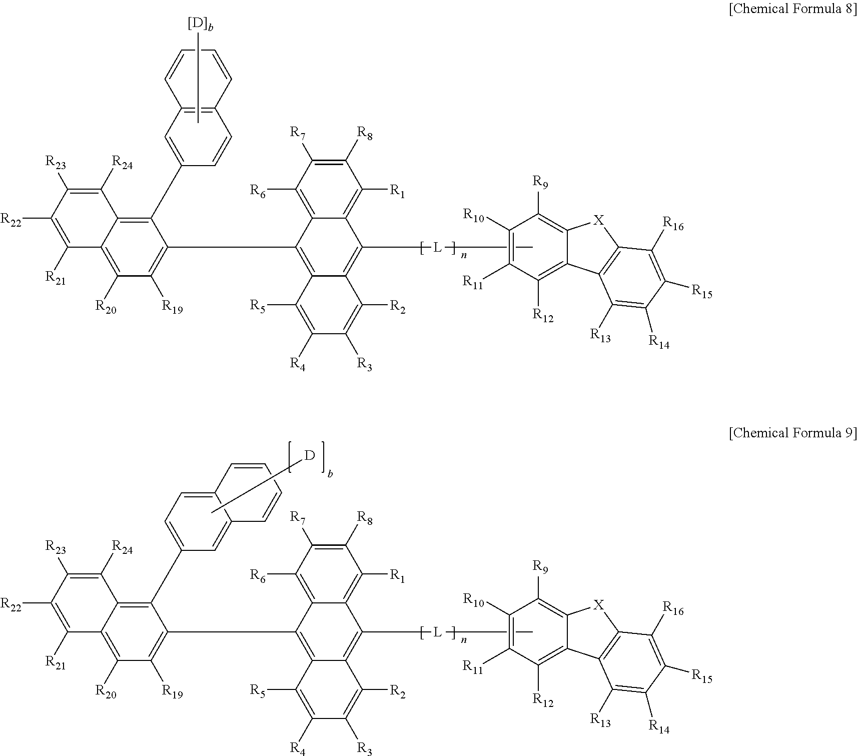

In Chemical Formulas 4 to 9,

X, L, R₁ to R₁₆, R₁₉ to R₂₄, n, a and b are each as defined in Chemical Formula 1.

The compounds represented by Chemical Formulas 4 to 9 may be further embodied as any one of Chemical Formulas 4a to 9a.

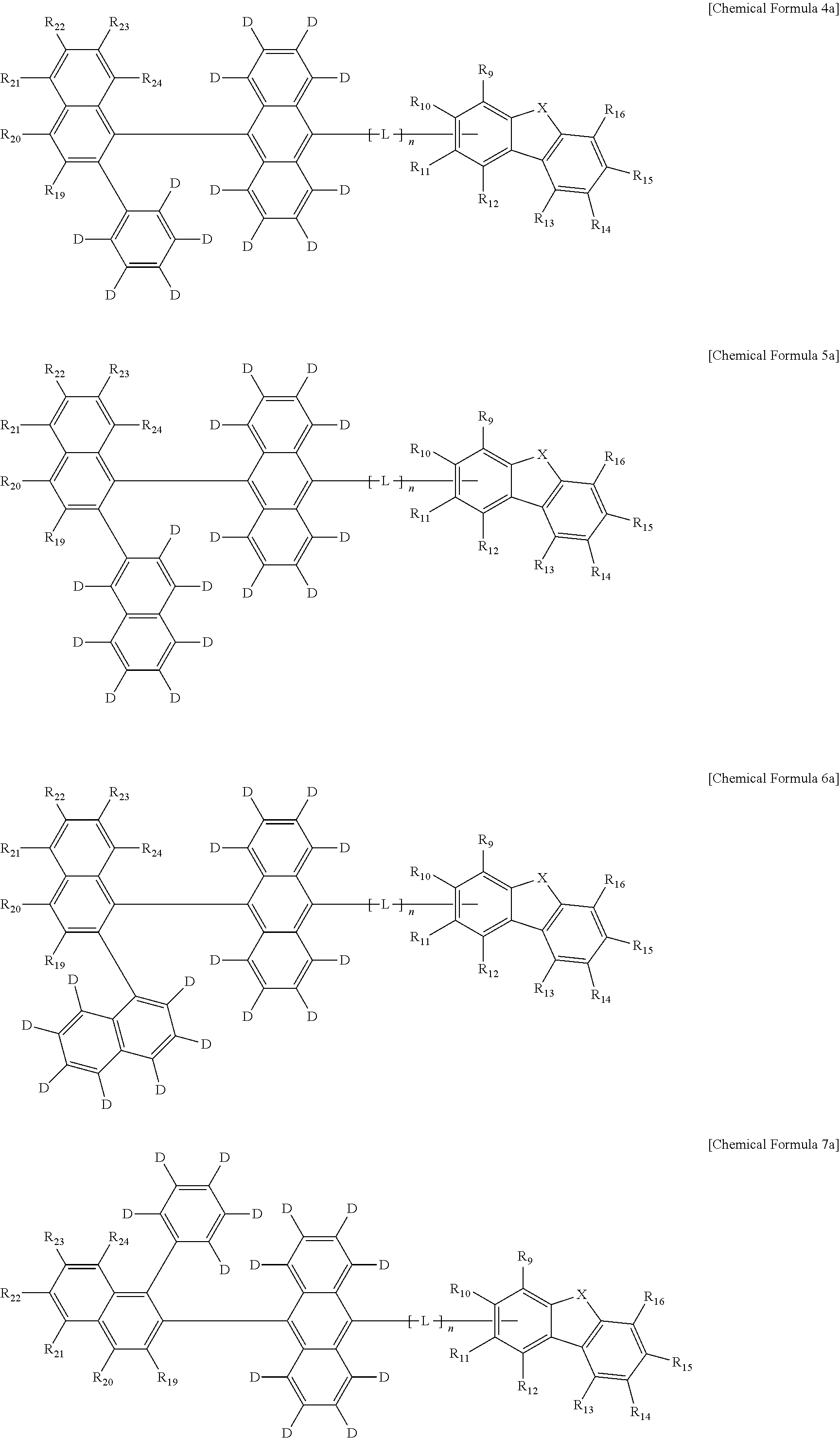

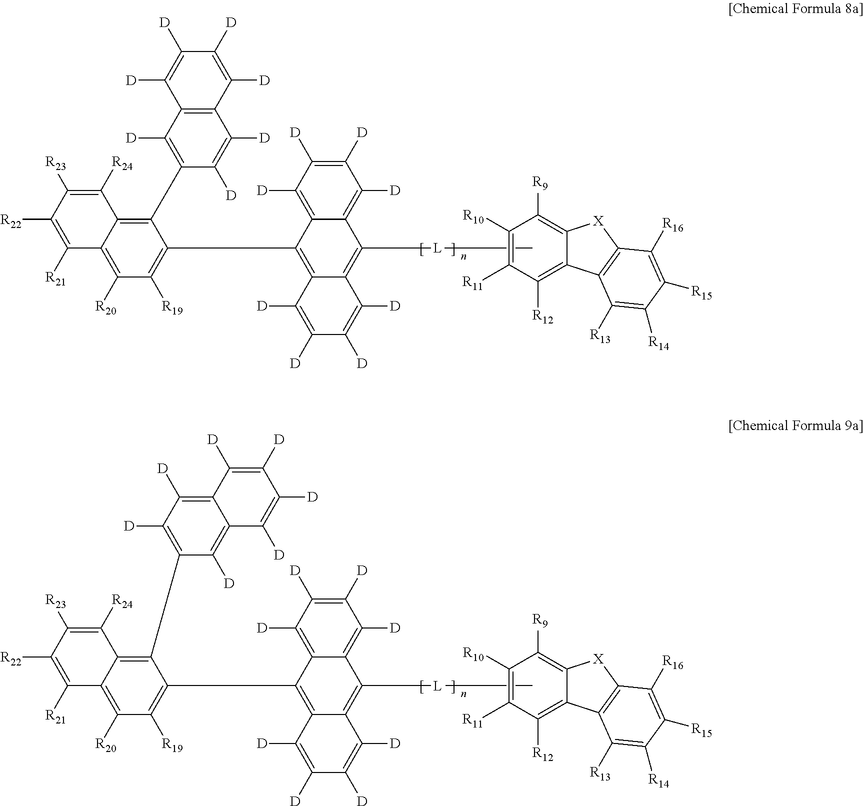

In another embodiment of the present invention, the compound represented by Chemical Formula 1 may be represented by any one of the following Chemical Formulas 10 to 13 according to a bonding position of the dibenzo-based moiety (e.g., X-containing ring).

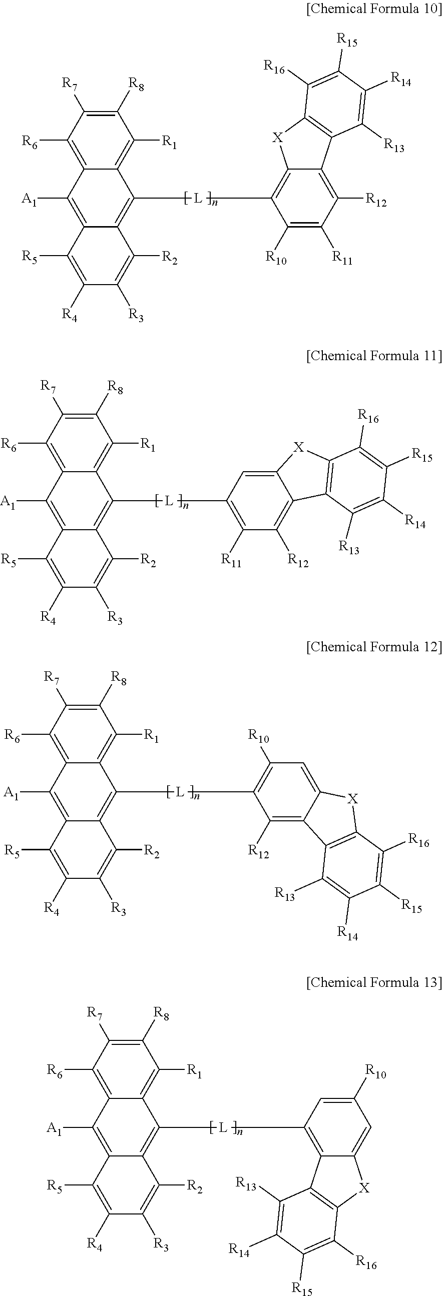

In Chemical Formulas 10 to 13,

A₁, X, L, R₁ to R₁₆, R₁₉ to R₂₄, n, a and b are each as defined in Chemical Formula 1.

In another embodiment of the present invention, the compound represented by Chemical Formula 1 may be represented by any one of the following Chemical Formulas 14 to 16 according to the type of the dibenzo-based moiety (e.g., X-containing ring).

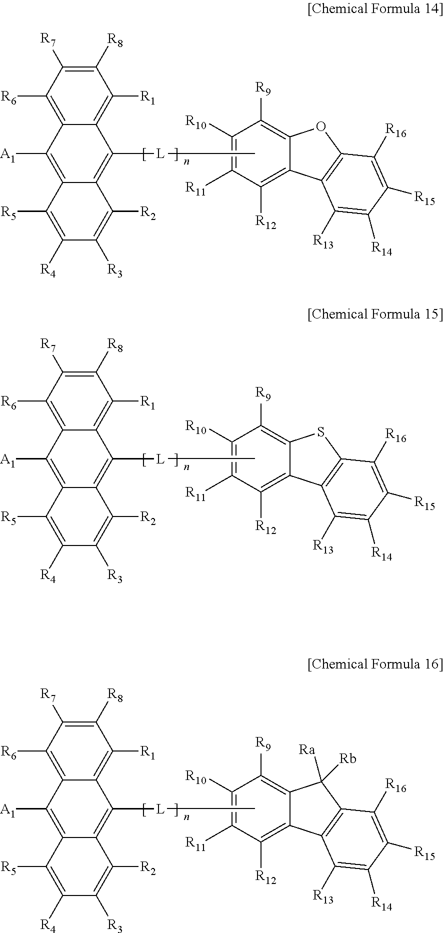

In Chemical Formulas 14 to 16,

A₁, X, L, R₁ to R₁₆, R₁₉ to R₂₄, Rₐ, R_{b}, and n are each as defined in Chemical Formula 1.

In another embodiment of the present invention, the compound represented by Chemical Formula 1 may be represented by Chemical Formula 17 or Chemical Formula 18 according to the type of substituent introduced into the dibenzo-based moiety (e.g., X-containing ring).

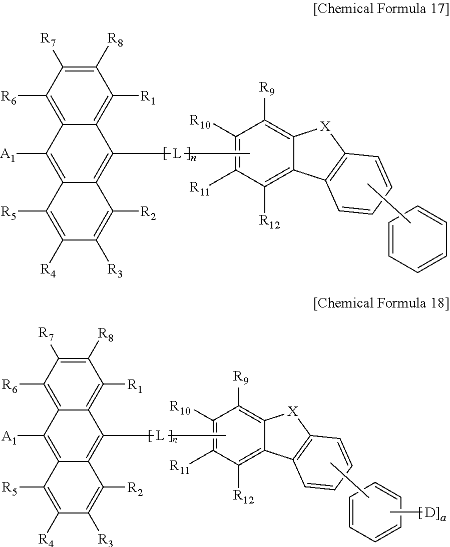

In Chemical Formula 17 or 18,

A₁, X, L, R₁ to R₁₂, and n are each as defined in Chemical Formula 1.

The compound represented by Chemical Formula 1 according to the present invention as described above may be further embodied as a compound represented by any one of Compounds 1 to 64 exemplified below. However, the compound represented by Chemical Formula 1 of the present invention is not limited to those exemplified below.

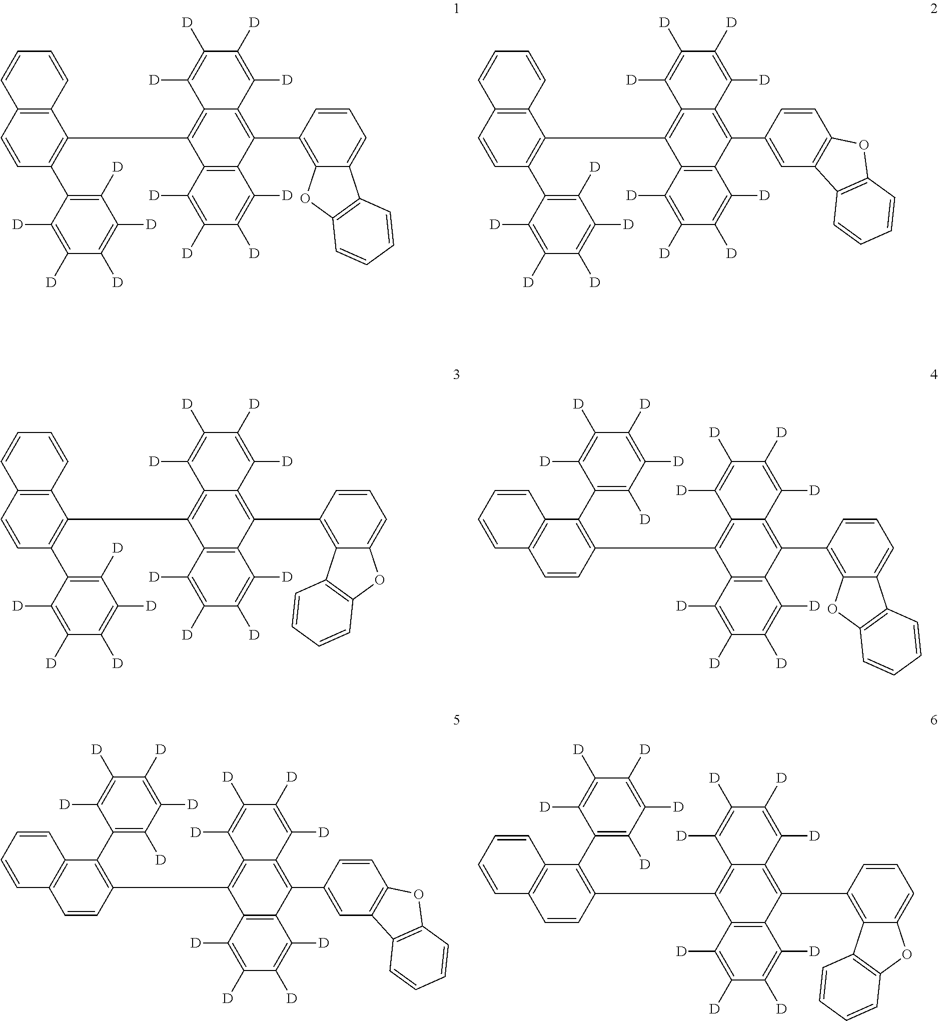

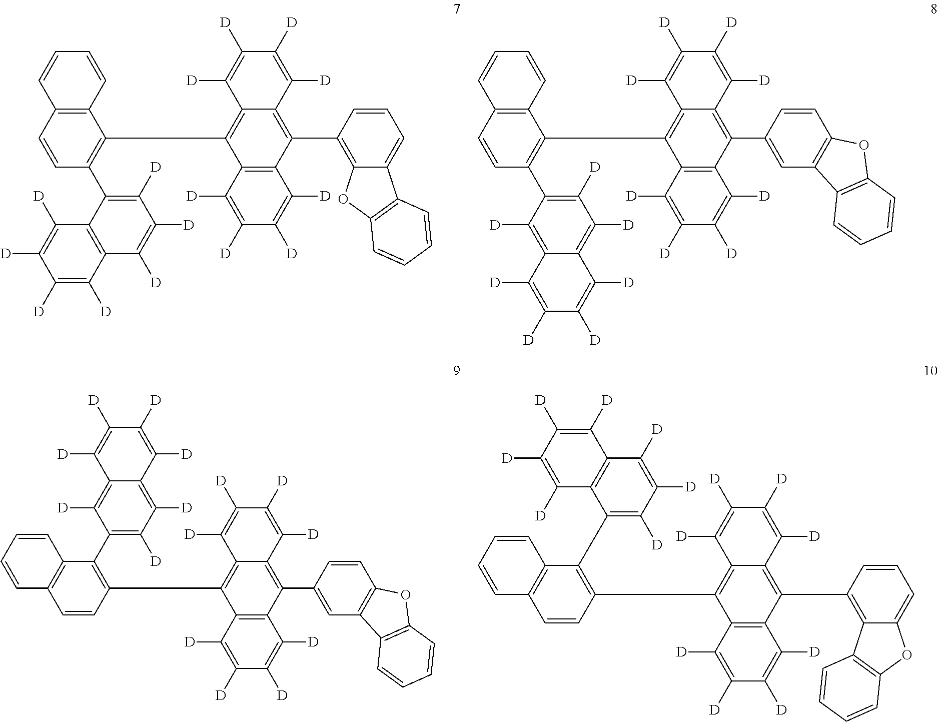

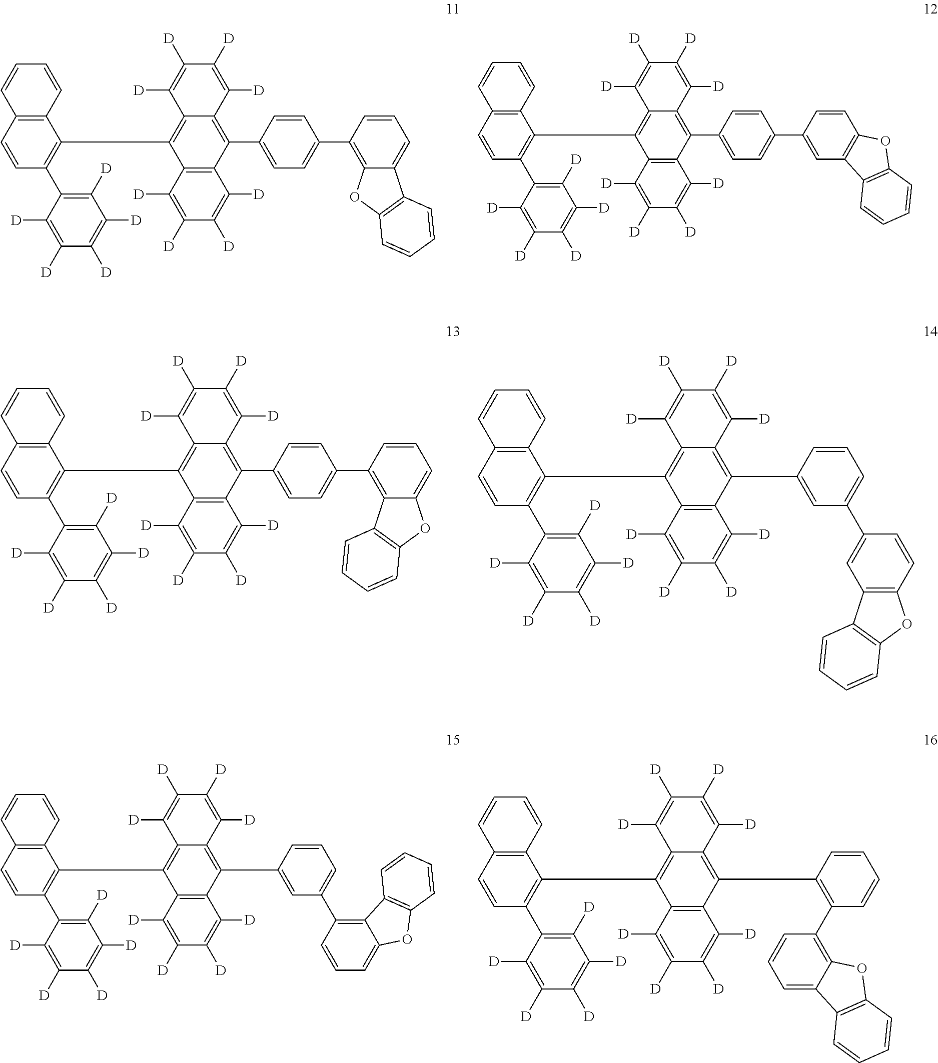

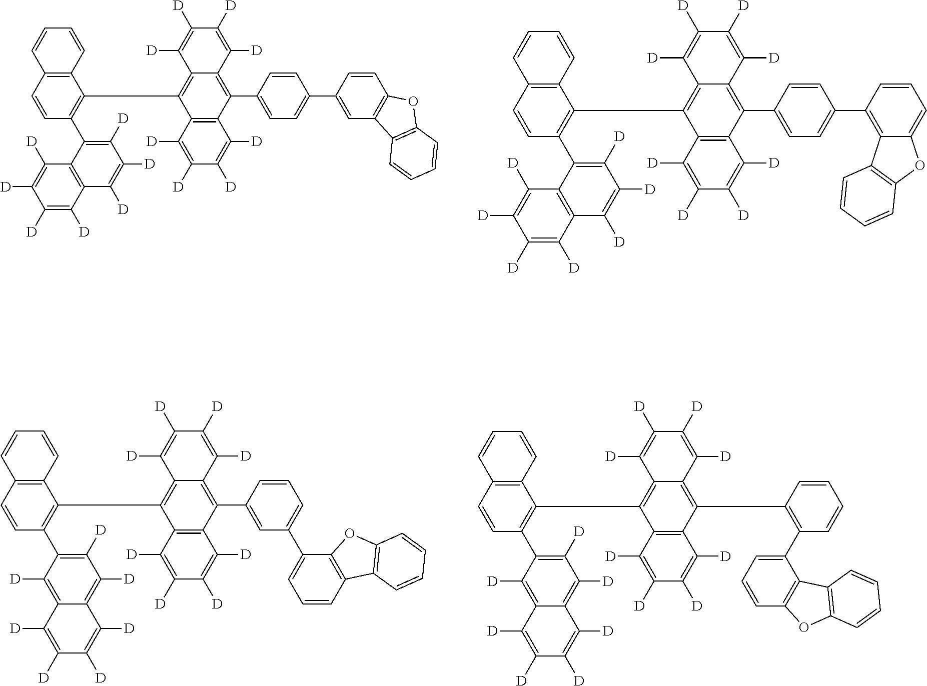

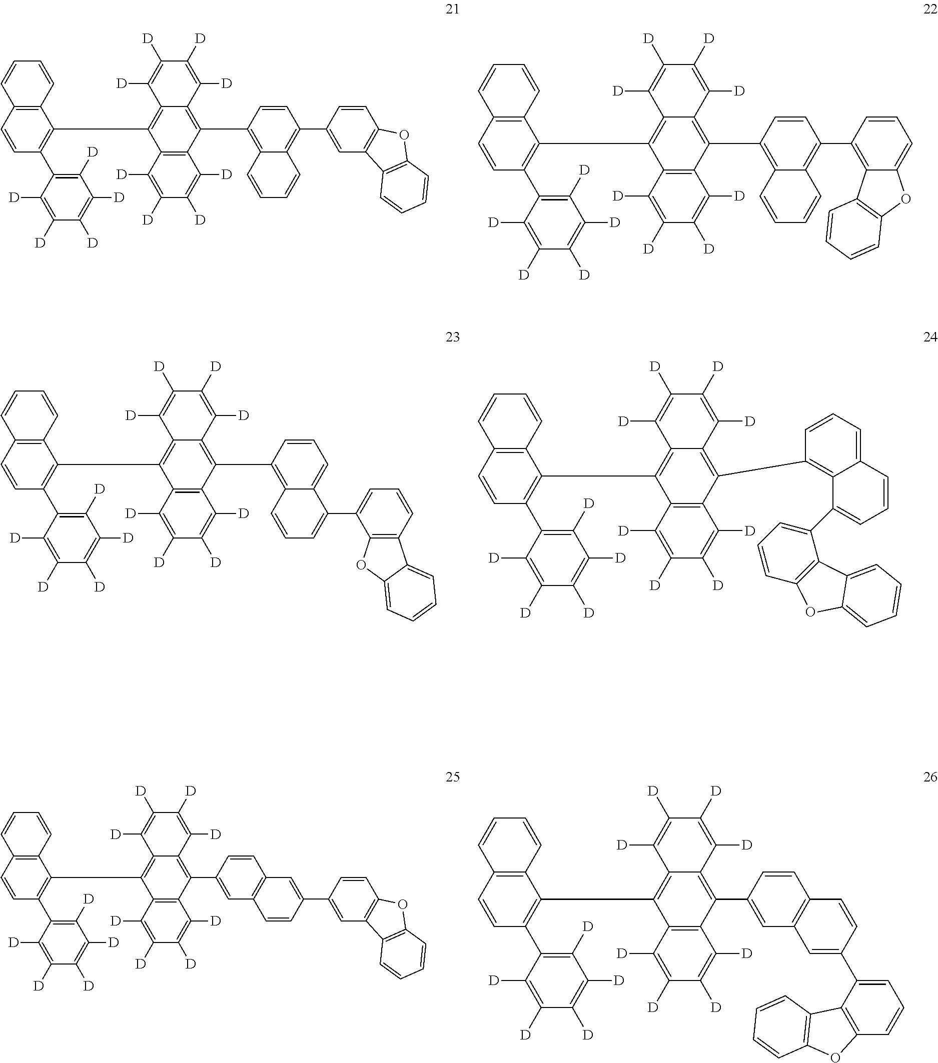

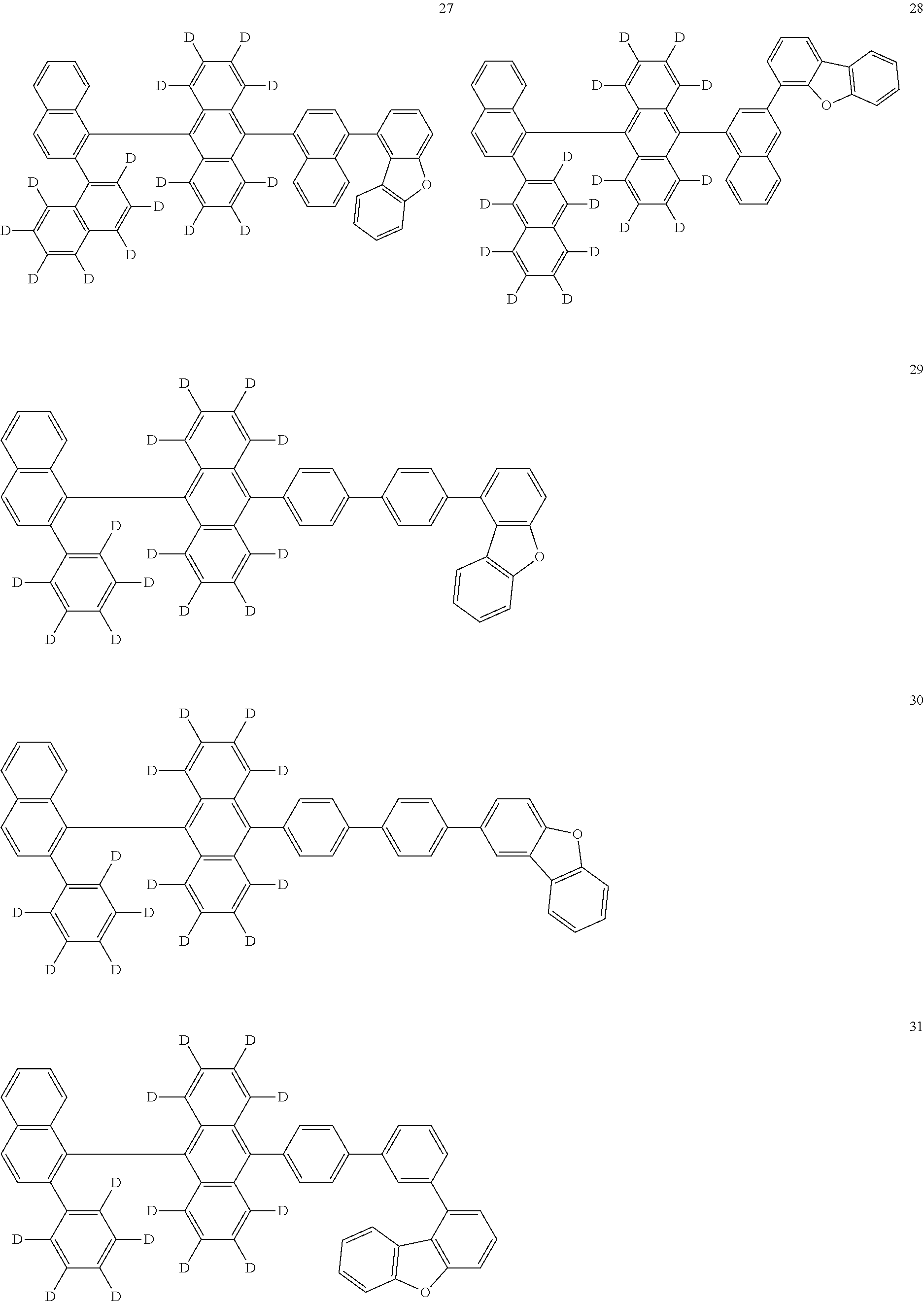

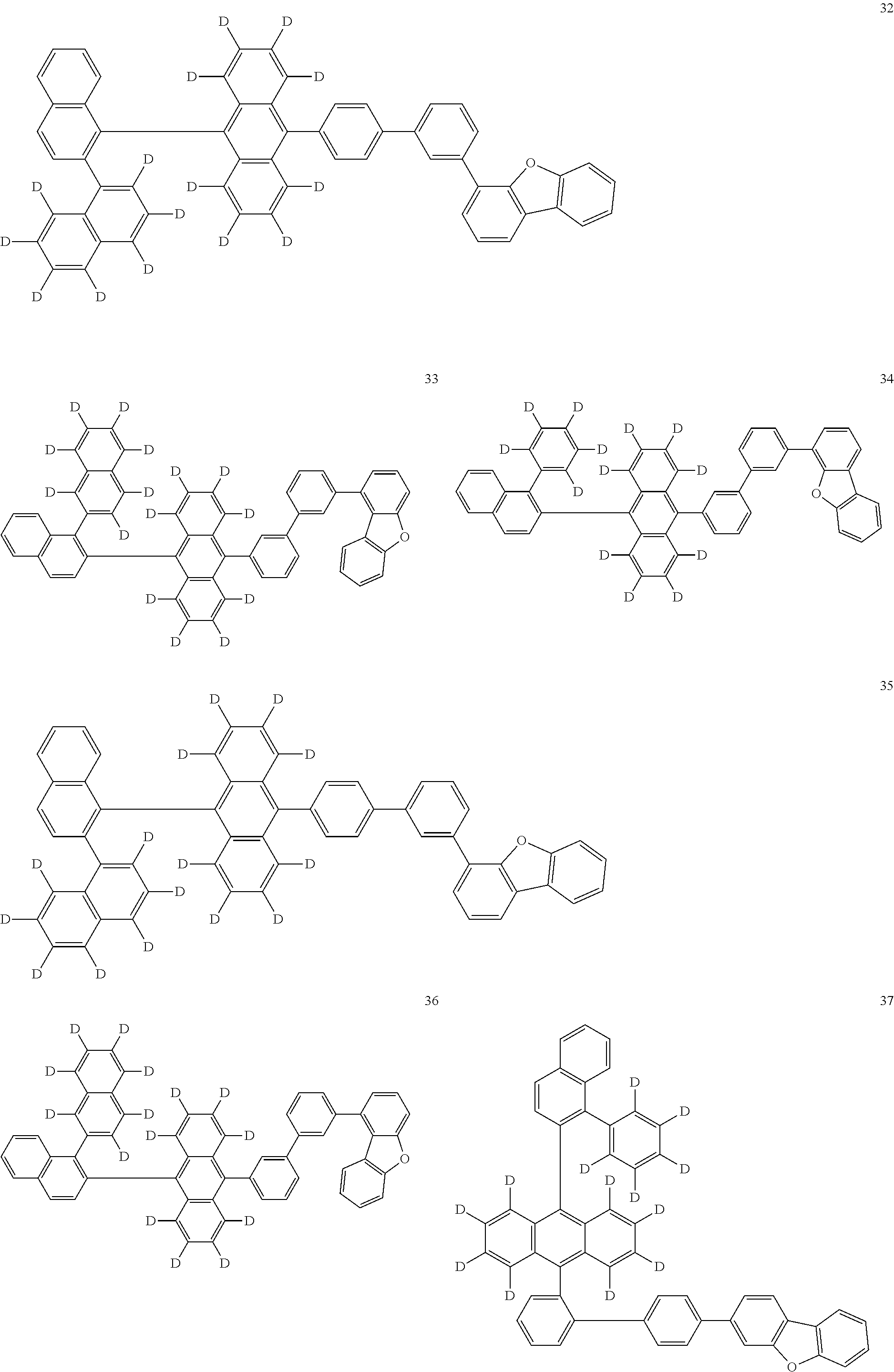

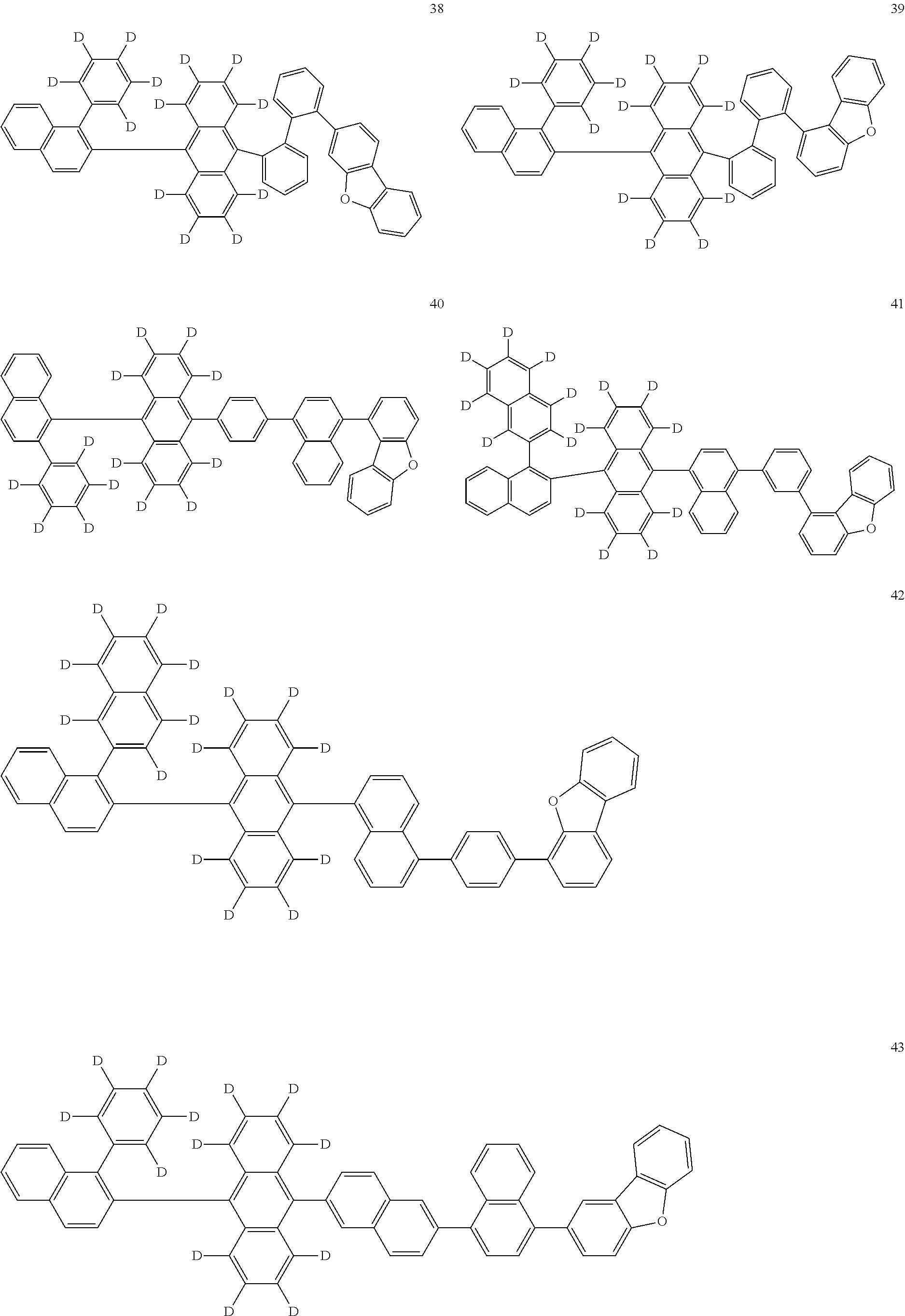

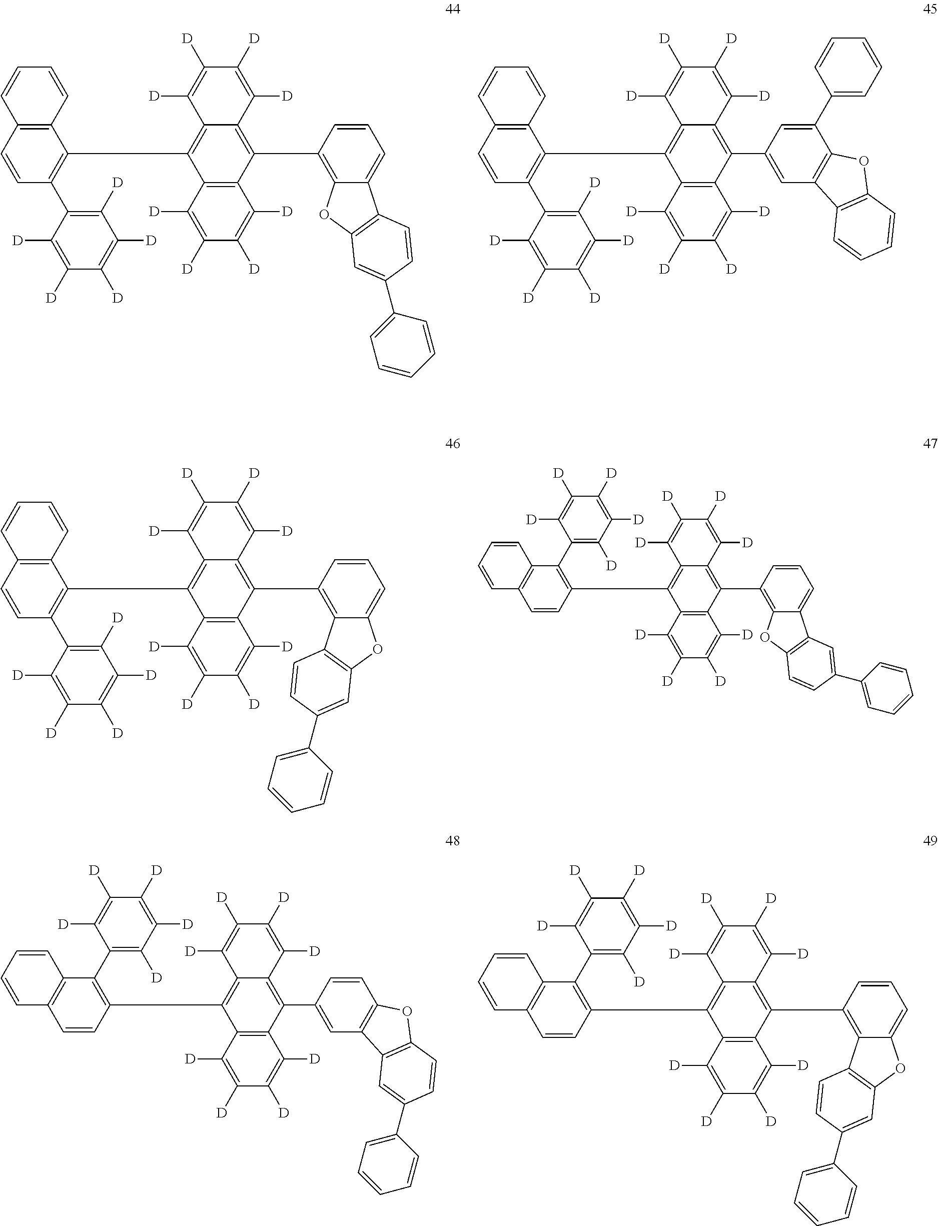

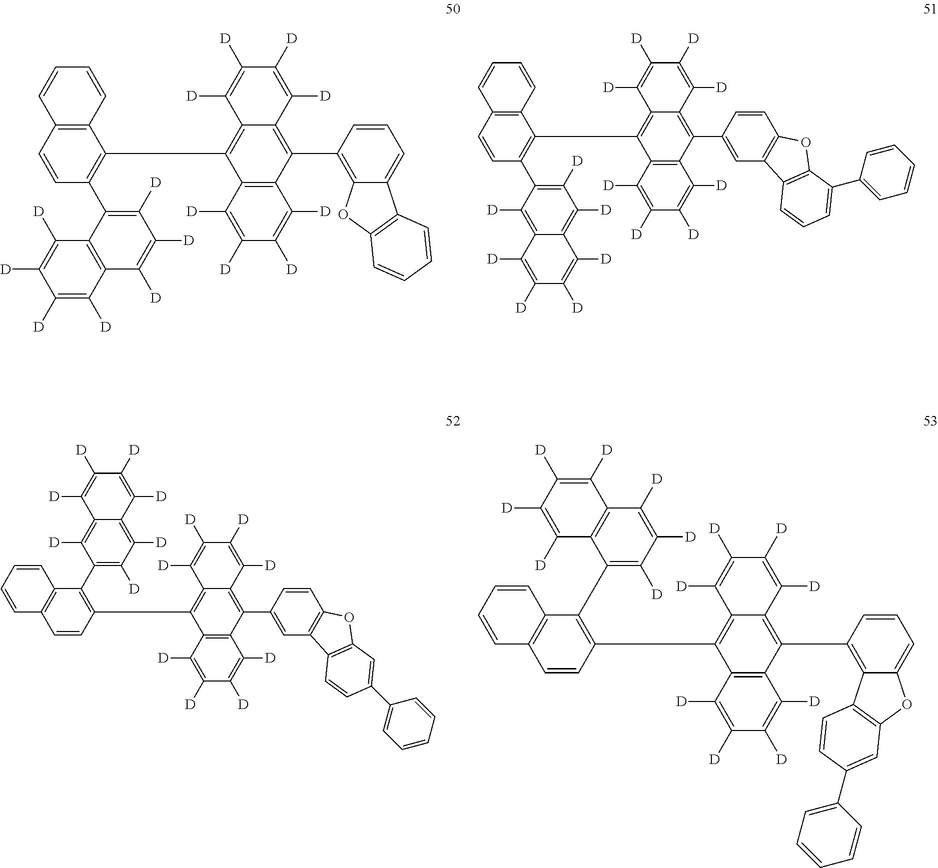

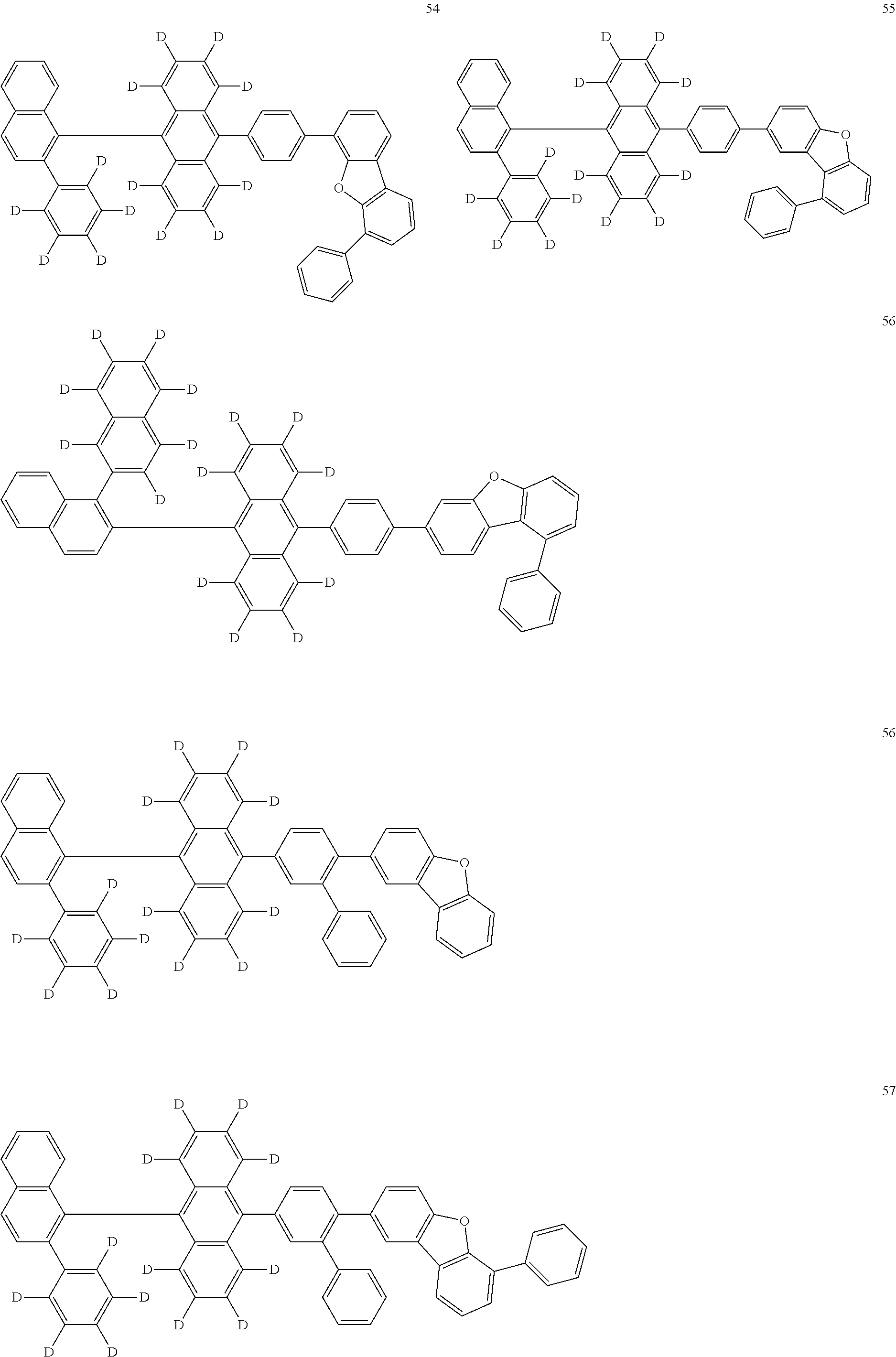

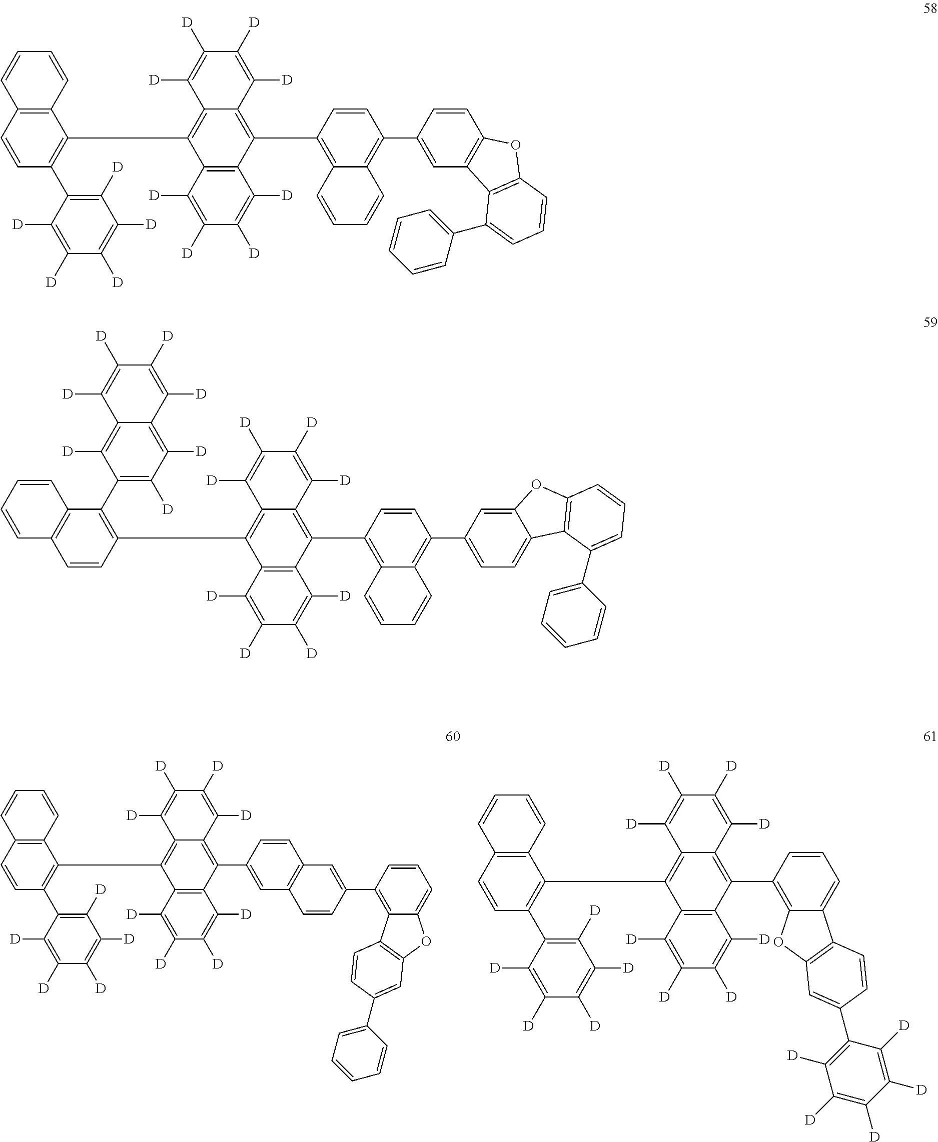

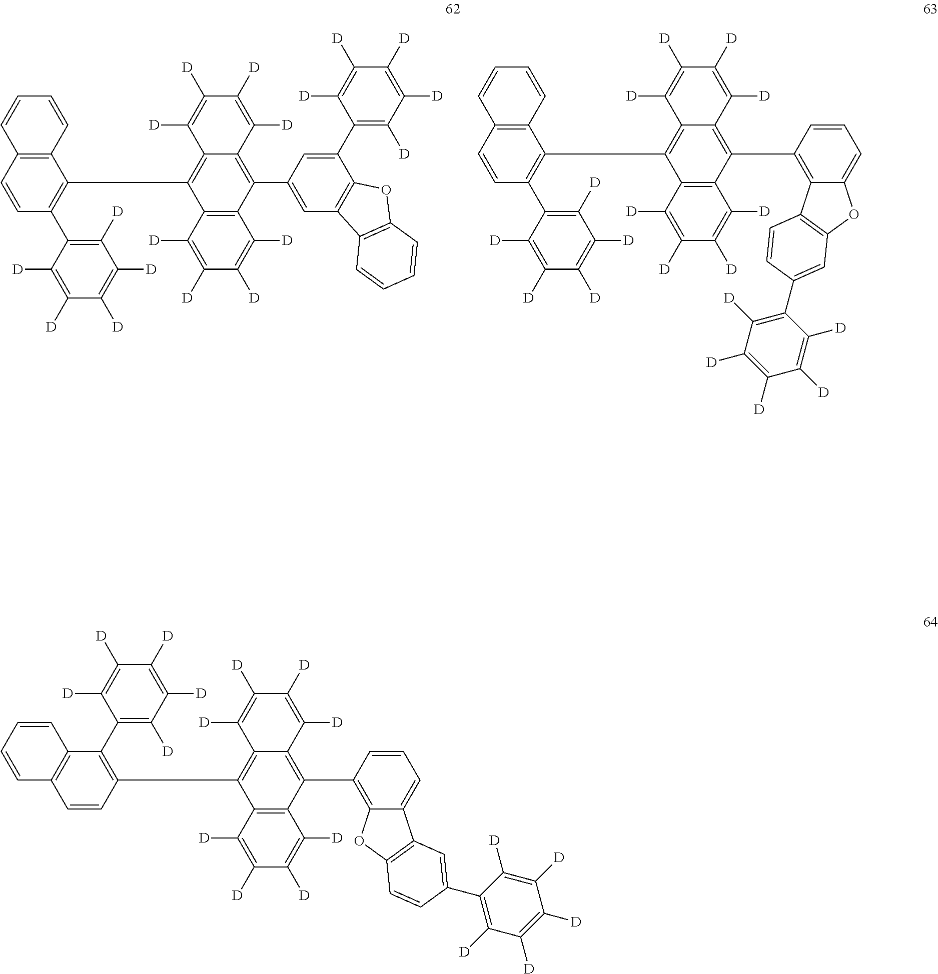

As used herein, “alkyl” refers to a monovalent substituent derived from a linear or branched chain saturated hydrocarbon having 1 to 40 carbon atoms. Examples of such alkyl may include, but not limited to, methyl, ethyl, propyl, isobutyl, sec-butyl, pentyl, iso-amyl, hexyl or the like.

As used herein, “alkenyl” refers to a monovalent substituent derived from a linear or branched chain unsaturated hydrocarbon having 2 to 40 carbon atoms, having at least one carbon-carbon double bond. Examples of such alkenyl may include, but not limited to, vinyl, allyl, isopropenyl, 2-butenyl or the like.

As used herein, “alkynyl” refers to a monovalent substituent derived from a linear or branched chain unsaturated hydrocarbon having 2 to 40 carbon atoms, having at least one carbon-carbon triple bond. Examples of such alkynyl may include, but not limited to, ethynyl, 2-propynyl or the like.

As used herein, “cycloalkyl” refers to a monovalent substituent derived from a monocyclic or polycyclic non-aromatic hydrocarbon having 3 to 40 carbon atoms. Examples of such cycloalkyl may include, but not limited to, cyclopropyl, cyclopentyl, cyclohexyl, norbornyl, adamantine or the like.

As used herein, “heterocycloalkyl” refers to a monovalent substituent derived from a non-aromatic hydrocarbon having 3 to 40 nuclear atoms, where one or more carbons in the ring, preferably one to three carbons, are substituted with a heteroatom such as N, O, S or Se. Examples of such heterocycloalkyl may include, but not limited to, morpholine, piperazine or the like.

As used herein, “aryl” refers to a monovalent substituent derived from a C₆ to C₄₀ aromatic hydrocarbon which is in a structure with a single ring or two or more rings combined with each other. In addition, a form in which two or more rings are pendant (e.g., simply attached) to or fused with each other may also be included. Examples of such aryl may include, but not limited to, phenyl, naphthyl, phenanthryl, anthryl or the like.

As used herein, “heteroaryl” refers to a monovalent substituent derived from a monoheterocyclic or polyheterocyclic aromatic hydrocarbon having 5 to 60 nuclear atoms. In such an embodiment, one or more carbons in the ring, preferably one to three carbons, are substituted with a heteroatom such as N, O, S or Se. In addition, a form in which two or more rings are pendant to or fused with each other may be included and a form fused with an aryl group may be included. Examples of such heteroaryl may include, but not limited to, a 6-membered monocyclic ring including, for example, pyridyl, pyrazinyl, pyrimidinyl, pyridazinyl and triazinyl; a polycyclic ring including, for example, phenoxathienyl, indolinzinyl, indolyl, purinyl, quinolyl, benzothiazole, and caR_{b}azolyl; 2-furanyl; N-imidazolyl; 2-isoxazolyl; 2-pyridinyl; 2-pyrimidinyl or the like.

As used herein, “alkyloxy” refers to a monovalent substituent represented by R′O—, where R′ refers to alkyl having 1 to 40 carbon atoms. Such alkyloxy may include a linear, branched or cyclic structure. Examples of such alkyloxy may include, but not limited to, methoxy, ethoxy, n-propoxy, 1-propoxy, t-butoxy, n-butoxy, pentoxy or the like.

As used herein, “aryloxy” is a monovalent substituent represented by RO—, where R refers to a C₆ to C₆₀ aryl. Examples of such aryloxy may include, but not limited to, phenyloxy, naphthyloxy, diphenyloxy or the like.

As used herein, “alkylsilyl” refers to silyl in which substitution with alkyl having 1 to 40 carbon atoms has been made, and “arylsilyl” refers to silyl in which substitution with a C₆ to C₆ aryl has been made.

As used herein, “alkyl boron” refers to boron substituted with a C₁ to C₄₀ alkyl, and “aryl boron” refers to boron substituted with a C₆ to C₆₀ aryl.

As used herein, “arylphosphine” refers to phosphine substituted with a C₆ to C₆₀ aryl, and “arylphosphine oxide group” refers to phosphine substituted with a C₆ to C₆₀ aryl and containing O.

As used herein, the term “condensed ring (e.g., fused ring)” refers to a condensed aliphatic ring, a condensed aromatic ring, a condensed heteroaliphatic ring, a condensed heteroaromatic ring, or a combination thereof.

As used herein, “arylamine” refers to amine substituted with a C₆ to C₆₀ aryl.

The compound represented by Chemical Formula 1 of the present invention may be prepared without limitation according to a method known in the art. As an example, it may be variously synthesized by referring to the synthesis process of the following examples.

<Organic Electroluminescent Element>

The present invention provides an organic electroluminescent element (“organic EL element”) including the compound represented by Chemical Formula 1.

More specifically, the organic EL element according to the present invention includes an anode (e.g., a positive electrode), a cathode (e.g., a negative electrode), and one or more organic layers disposed between the anode and the cathode, where at least one of the one or more organic layers includes the compound represented by Chemical Formula 1. In such a case, the compound may be used alone or in combination of two or more kinds thereof.

The one or more organic layers may be any one or more of a hole injection layer, a hole transport layer, a light emitting auxiliary layer, a light emitting layer, an electron transport layer, and an electron injection layer, and at least one of the organic layers may include the compound represented by Chemical Formula 1. Specifically, the organic layer including the compound represented by Chemical Formula 1 may be a light emitting layer, and more specifically, may be a blue fluorescent light emitting layer material.

In the light emitting layer of the organic EL element, a triplet energy gap of a host of a host material should be higher than that of a dopant. That is, in order to effectively provide phosphorescent light emission from the dopant, a lowest excited state of the host should have a higher energy than a lowest discharge state (e.g., emission state) of the dopant. The compound represented by Chemical Formula 1 has a high triplet energy and may be used as a host material because its energy level may be adjusted to be higher than that of the dopant. The compound represented by Chemical Formula 1 may prevent excitons generated in the light emitting layer from being diffused into an electron transport layer or a hole transport layer which are adjacent to the light emitting layer. Accordingly, the number of excitons contributing to light emission in the light emitting layer may be increased such that luminescence efficiency of the element may be improved and durability and stability of the element may be improved to effectively improve the lifespan of the element as well.

The light emitting layer of the organic EL element of the present invention includes a host material and a dopant material, and in such a case, the compound of Chemical Formula 1 may be used as a fluorescent host material. In addition to the host material of Chemical Formula 1 described above, the light emitting layer may include conventional hosts and/or dopants known in the art without limitation. Their content ratio (mixing ratio) is not particularly limited and may be appropriately adjusted within a content range known in the art.

A structure of the organic EL element of the present invention is not particularly limited, and a non-limiting example may be a structure in which a substrate, an anode, a hole injection layer, a hole transport layer, a light emitting auxiliary layer, a light emitting layer, an electron transport layer, an electron injection layer, and a cathode are sequentially stacked. In such a case, at least one of the hole injection layer, the hole transport layer, the light emitting auxiliary layer, the light emitting layer, the electron transport layer and the electron injection layer may include the compound represented by Chemical Formula 1, and preferably, the light emitting layer may include the compound represented by Chemical Formula 1. The structure of the organic EL element of the present invention may have a structure in which an insulating layer or an adhesive layer is inserted at an interface between the electrode and the organic layer.

The organic EL element of the present invention may be prepared using materials and methods known in the art, except that one or more layers of the aforementioned organic layers include the compound represented by Chemical Formula 1.

The organic layer may be formed by a vacuum deposition method or a solution coating method. Examples of the solution coating method may include, but not limited to, spin coating, dip coating, doctor blading, inkjet printing, thermal transfer or the like.

The substrate used in preparation of the organic EL element of the present invention is not particularly limited, and non-limiting examples thereof may include silicon wafers, quartz, glass plates, metal plates, plastic films, sheets or the like.

In addition, examples of an anode material may include, but not limited to, a metal such as vanadium, chromium, copper, zinc, and gold or an alloy thereof; metal oxides such as zinc oxide, indium oxide, indium tin oxide (ITO), or indium zinc oxide (IZO); combination of oxide with metal such as ZnO:Al or SnO₂:Sb; conductive polymers such as polythiophene, poly(3-methylthiophene), poly [3,4-(ethylene-1,2-dioxy) thiophene](PEDT), polypyrrole or polyaniline; carbon black or the like.

In addition, examples of a cathode material may include, but not limited to, a metal such as magnesium, calcium, sodium, potassium, titanium, indium, yttrium, lithium, gadolinium, aluminum, silver, tin, or lead or an alloy thereof; a multi-layered material such as LiF/Al or LiO₂/Al or the like.

In addition, materials for the hole injection layer, the light emitting layer, the electron injection layer, and the electron transport layer are not particularly limited and conventional materials known in the art may be used without limitation.

Hereinafter, the present invention will be described in detail with reference to the following examples. However, the following examples are merely to illustrate the invention, and the present invention is not limited to the following embodiments.

### PREPARATION EXAMPLE

### [Preparation Example 1] Synthesis of Core 1

<Step 1> Synthesis of Core 1-1

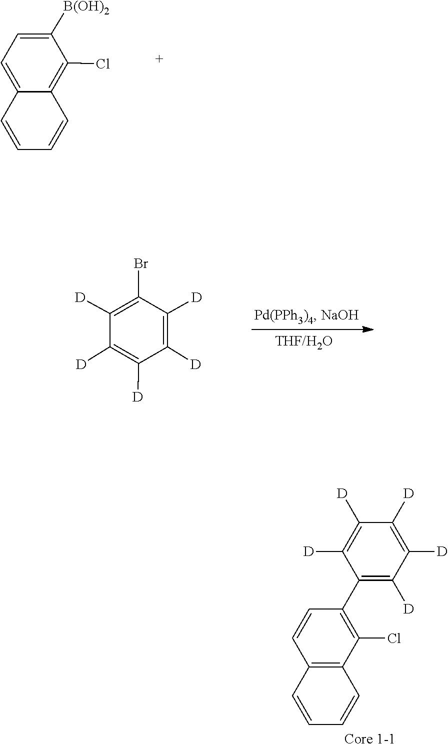 [SMILES: OB(O)c1ccc2ccccc2c1Cl ; [2H]c1c([2H])c([2H])c(Br)c([2H])c1[2H] ; [2H]c1c([2H])c([2H])c(-c2ccc3ccccc3c2Cl)c([2H])c1[2H]]

(1-chloronaphthalen-2-yl)boronic acid (41 g, 200 mmol) and 1-bromobenzene-2,3,4,5,6-d5 (32 g, 200 mmol), Pd(PPh₃)₄ (9 g, 8 mmol) and NaOH (24 g, 600 mmol) were added to 1,000 ml of THF and 500 ml of H₂O and stirred at 80° C. for 8 hours. After completion of the reaction, 500 ml of water was added thereto and stirred. After completion of the reaction, an organic layer was separated through extraction and then concentrated. The concentrated organic layer was dissolved in toluene, followed by silica filtering, and then recrystallized with ethanol to obtain the target compound, Core 1-1 (42 g, yield 88%).

¹H-NMR: δ 7.51 (t, 1H), 7.59 (t, 1H), 7.62 (d, 1H), 7.88 (d, 1H), 8.24 (d, 1H), 8.25 (d, 1H)

<Step 2> Synthesis of Core 1-2

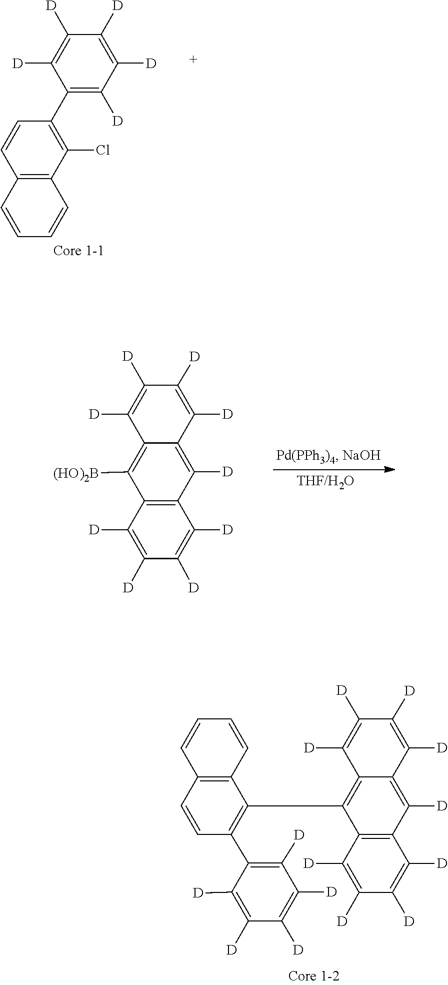 [SMILES: [2H]c1c([2H])c([2H])c(-c2ccc3ccccc3c2Cl)c([2H])c1[2H] ; [2H]c1c([2H])c([2H])c2c(B(O)O)c3c([2H])c([2H])c([2H])c([2H])c3c([2H])c2c1[2H] ; [2H]c1c([2H])c([2H])c(-c2ccc3ccccc3c2-c2c3c([2H])c([2H])c([2H])c([2H])c3c([2H])c3c([2H])c([2H])c([2H])c([2H])c23)c([2H])c1[2H]]

Core 1-1 (37 g, 150 mmol), (anthracen-9-yl-d9)boronic acid (35 g, 150 mmol), Pd(PPh₃)₄ (7 g, 6 mmol), and NaOH (18 g, 450 mmol) were added to 500 ml of THF and 250 ml of H₂O and stirred at 80° C. for 8 hours. After completion of the reaction, 200 ml of water was added thereto and stirred. After completion of the reaction, an organic layer was separated through extraction and then concentrated. The concentrated organic layer was dissolved in toluene, followed by silica filtering, and then recrystallized with ethanol to obtain the target compound, Core 1-2 (47 g, yield 79%).

¹H-NMR: δ 7.40 (t, 1H), 7.54 (t, 1H), 7.72 (d, 1H), 8.26 (d, 1H), 8.31 (d, 1H), 8.93 (d, 1H)

<Step 3> Synthesis of Core 1

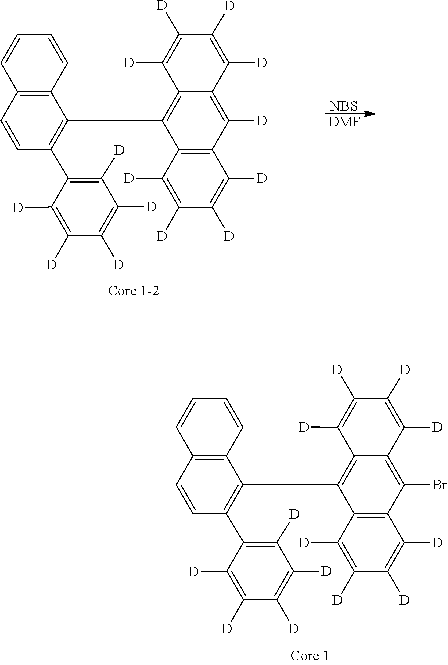

Core 1-2 (39 g, 100 mmol) and N-Bromosuccinimide (18 g, 100 mmol) were dissolved in 400 ml of dimethyl formamide, and a mixture thereof was then stirred at room temperature for 4 hours. After completion of the reaction, 400 ml of water was added thereto and a resultant solid was filtered. The filtered solid was dissolved in dichloromethane, and then filtered with silica. The organic solvent was concentrated, and then recrystallized with ethanol to obtain the target compound, Core 1 (40 g, yield 93%).

¹H-NMR: δ 7.40 (t, 1H), 7.54 (t, 1H), 7.72 (d, 1H), 8.26 (d, 1H), 8.31 (d, 1H), 8.93 (d, 1H)

### [Preparation Example 2] Synthesis of Core 2

<Step 1> Synthesis of Core 2-1

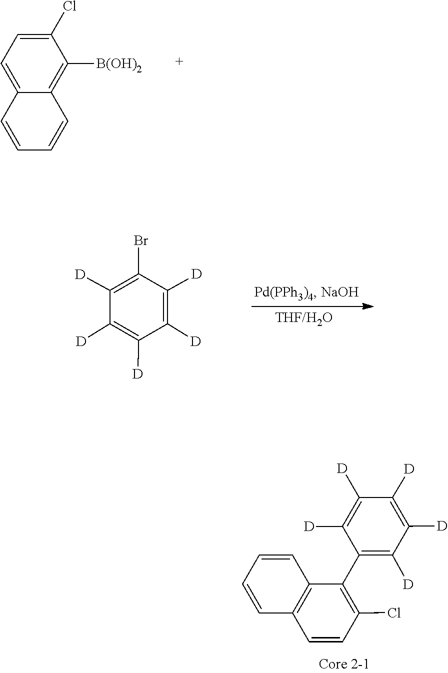 [SMILES: OB(O)c1c(Cl)ccc2ccccc12 ; [2H]c1c([2H])c([2H])c(Br)c([2H])c1[2H] ; [2H]c1c([2H])c([2H])c(-c2c(Cl)ccc3ccccc23)c([2H])c1[2H]]

(2-chloronaphthalen-1-yl)boronic acid (41 g, 200 mmol), 1-bromobenzene-2,3,4,5,6-d5 (32 g, 200 mmol), Pd(PPh₃)₄ (9 g, 8 mmol), and NaOH (24 g, 600 mmol) were added to 1,000 ml of THF and 500 ml of H₂O and stirred at 80° C. for 8 hours. After completion of the reaction, 500 ml of water was added thereto and stirred. After completion of the reaction, an organic layer was separated through extraction and then concentrated. The concentrated organic layer was dissolved in toluene, followed by silica filtering, and then recrystallized with ethanol to obtain the target compound, Core 2-1 (41 g, yield 85%).

¹H-NMR: δ 7.34 (t, 1H), 7.44 (d, 2H), 7.49 (t, 1H), 7.98 (d, 1H), 8.10 (d, 1H), 8.94 (d, 1H)

<Step 2> Synthesis of Core 2-2

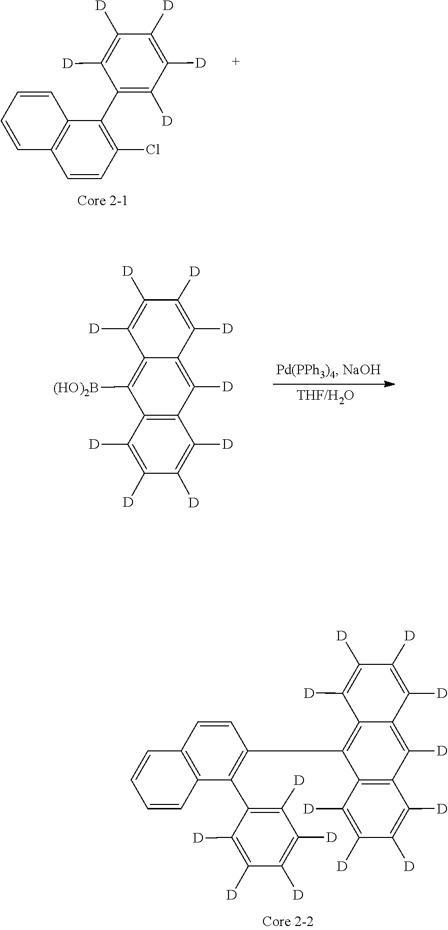 [SMILES: [2H]c1c([2H])c([2H])c(-c2c(Cl)ccc3ccccc23)c([2H])c1[2H] ; [2H]c1c([2H])c([2H])c2c(B(O)O)c3c([2H])c([2H])c([2H])c([2H])c3c([2H])c2c1[2H] ; [2H]c1c([2H])c([2H])c(-c2c(-c3c4c([2H])c([2H])c([2H])c([2H])c4c([2H])c4c([2H])c([2H])c([2H])c([2H])c34)ccc3ccccc23)c([2H])c1[2H]]

Core 2-1 (37 g, 150 mmol, (anthracen-9-yl-d9)boronic acid (35 g, 150 mmol), Pd(PPh₃)₄ (7 g, 6 mmol), and NaOH (18 g, 450 mmol) were added to 500 ml of THF and 250 ml of H₂O and stirred at 80° C. for 8 hours. After completion of the reaction, 200 ml of water was added thereto and stirred. After completion of the reaction, an organic layer was separated through extraction and then concentrated. The concentrated organic layer was dissolved in toluene, followed by silica filtering, and then recrystallized with ethanol to obtain the target compound, Core 2-2 (45 g, yield 76%).

¹H-NMR: δ 7.40 (t, 1H), 7.54 (t, 1H), 7.72 (d, 1H), 8.26 (d, 1H), 8.31 (d, 1H), 8.93 (d, 1H)

<Step 3> Synthesis of Core 2

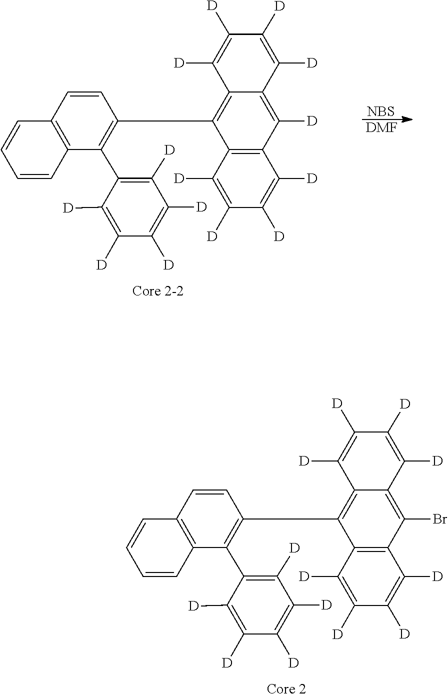

Core 2-2 (39 g, 100 mmol) and N-Bromosuccinimide (18 g, 100 mmol) were dissolved in 400 ml of dimethyl formamide, and a mixture thereof was then stirred at room temperature for 4 hours. After completion of the reaction, 400 ml of water was added thereto and a resultant solid was filtered. The filtered solid was dissolved in dichloromethane, and then filtered with silica. The organic solvent was concentrated, and then recrystallized with ethanol to obtain the target compound, Core 2 (41 g, yield 96%).

¹H-NMR: δ 7.40 (t, 1H), 7.54 (t, 1H), 7.72 (d, 1H), 8.26 (d, 1H), 8.31 (d, 1H), 8.93 (d, 1H)

### [Preparation Example 3] Synthesis of Core 3

<Step 1> Synthesis of Core 3-1

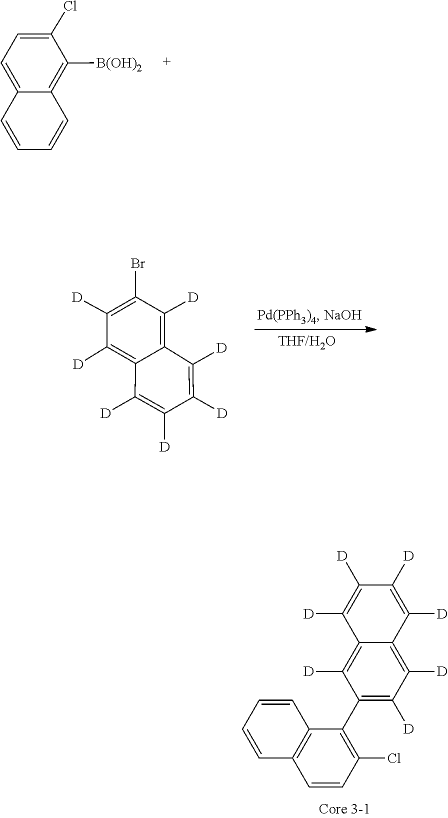 [SMILES: OB(O)c1c(Cl)ccc2ccccc12 ; [2H]c1c([2H])c([2H])c2c([2H])c(Br)c([2H])c([2H])c2c1[2H] ; [2H]c1c([2H])c([2H])c2c([2H])c(-c3c(Cl)ccc4ccccc34)c([2H])c([2H])c2c1[2H]]

(2-chloronaphthalen-1-yl)boronic acid (41 g, 200 mmol), 2-bromonaphthalene-1,3,4,5,6,7,8-d7 (43 g, 200 mmol), Pd(PPh₃)₄ (9 g, 8 mmol), and NaOH (24 g, 600 mmol) were added to 1,000 ml of THF and 500 ml of H₂O and stirred at 80° C. for 8 hours. After completion of the reaction, 500 ml of water was added thereto and stirred. After completion of the reaction, an organic layer was separated through extraction and then concentrated. The concentrated organic layer was dissolved in toluene, silica filtered, and recrystallized with ethanol to obtain the target compound, Core 3-1 (50 g, yield 85%).

¹H-NMR: δ 7.34 (t, 1H), 7.44 (d, 2H), 7.49 (t, 1H), 7.98 (d, 1H), 8.10 (d, 1H), 8.94 (d, 1H)

<Step 2> Synthesis of Core 3-2

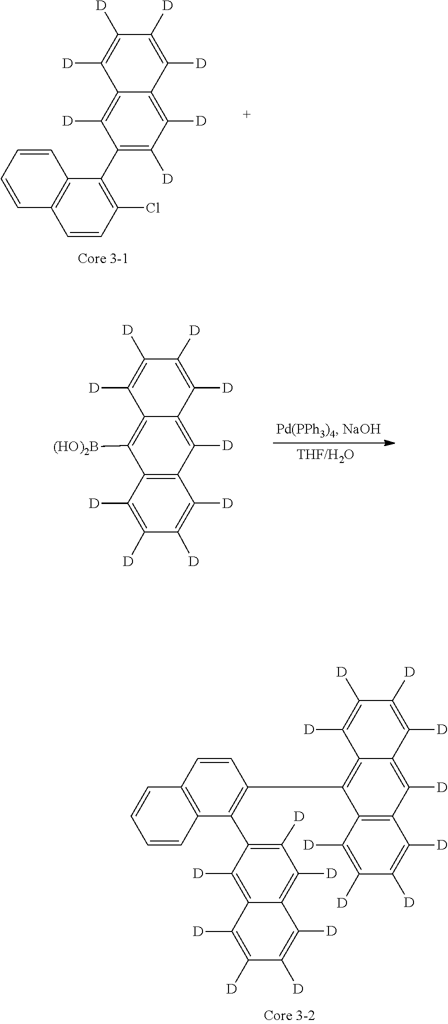 [SMILES: [2H]c1c([2H])c([2H])c2c([2H])c(-c3c(Cl)ccc4ccccc34)c([2H])c([2H])c2c1[2H] ; [2H]c1c([2H])c([2H])c2c(B(O)O)c3c([2H])c([2H])c([2H])c([2H])c3c([2H])c2c1[2H] ; [2H]c1c([2H])c([2H])c2c([2H])c(-c3c(-c4c5c([2H])c([2H])c([2H])c([2H])c5c([2H])c5c([2H])c([2H])c([2H])c([2H])c45)ccc4ccccc34)c([2H])c([2H])c2c1[2H]]

Core 3-1 (44 g, 150 mmol), (anthracen-9-yl-d9)boronic acid (35 g, 150 mmol), Pd(PPh₃)₄ (7 g, 6 mmol), and NaOH (18 g, 450 mmol) were added to 500 ml of THF and 250 ml of H₂O and stirred at 80° C. for 8 hours. After completion of the reaction, 200 ml of water was added thereto and stirred. After completion of the reaction, an organic layer was separated through extraction and then concentrated. The concentrated organic layer was dissolved in toluene, filtered with silica, and recrystallized with ethanol to obtain the target compound, Core 3-2 (50 g, yield 75%).

¹H-NMR: δ 7.40 (t, 1H), 7.54 (t, 2H), 7.72 (d, 1H), 8.26 (d, 1H), 7.31 (d, 1H), 8.93 (d, 1H)

<Step 3> Synthesis of Core 3

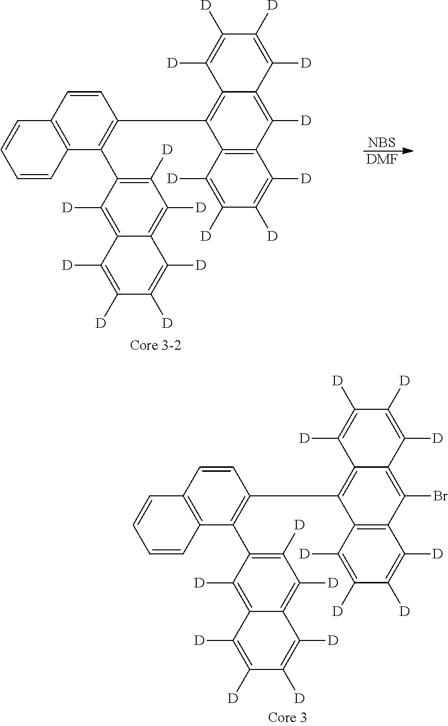

Core 3-2 (45 g, 100 mmol) and N-Bromosuccinimide (18 g, 100 mmol) were dissolved in 400 ml of dimethyl formamide, and a mixture thereof was then stirred at room temperature for 4 hours. After completion of the reaction, 400 ml of water was added thereto and a resultant solid was filtered. The filtered solid was dissolved in dichloromethane, and then filtered with silica. The organic solvent was concentrated, and then recrystallized with ethanol to obtain the target compound, Core 3 (47 g, yield 89%).

¹H-NMR: δ 7.40 (t, 1H), 7.54 (t, 2H), 7.72 (d, 1H), 8.26 (d, 1H), 7.31 (d, 1H), 8.93 (d, 1H)

### [Preparation Example 4] Synthesis of Core 4

<Step 1> Synthesis of Core 4-1

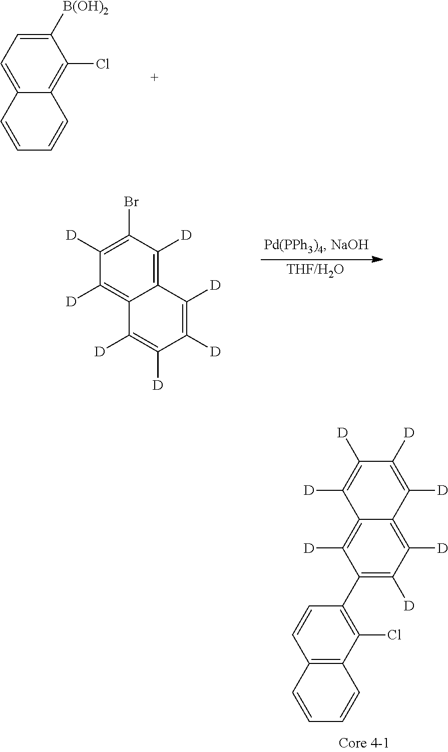 [SMILES: OB(O)c1ccc2ccccc2c1Cl ; [2H]c1c([2H])c([2H])c2c([2H])c(Br)c([2H])c([2H])c2c1[2H] ; [2H]c1c([2H])c([2H])c2c([2H])c(-c3ccc4ccccc4c3Cl)c([2H])c([2H])c2c1[2H]]

(1-chloronaphthalen-2-yl)boronic acid (37 g, 150 mmol), 2-bromonaphthalene-1,3,4,5,6,7,8-d7 (43 g, 200 mmol), Pd(PPh₃)₄ (7 g, 6 mmol), and NaOH (18 g, 450 mmol) were added to 500 ml of THF and 250 ml of H₂O and stirred at 80° C. for 8 hours. After completion of the reaction, 200 ml of water was added thereto and stirred. After completion of the reaction, an organic layer was separated through extraction and then concentrated. The concentrated organic layer was dissolved in toluene, filtered with silica, and recrystallized with ethanol to obtain the target compound, Core 4-1 (50 g, yield 85%).

¹H-NMR: δ 7.51 (t, 1H), 7.59 (t, 1H), 7.62 (d, 1H), 7.88 (d, 1H), 8.24 (d, 1H), 8.25 (d, 1H)

<Step 2> Synthesis of Core 4-2

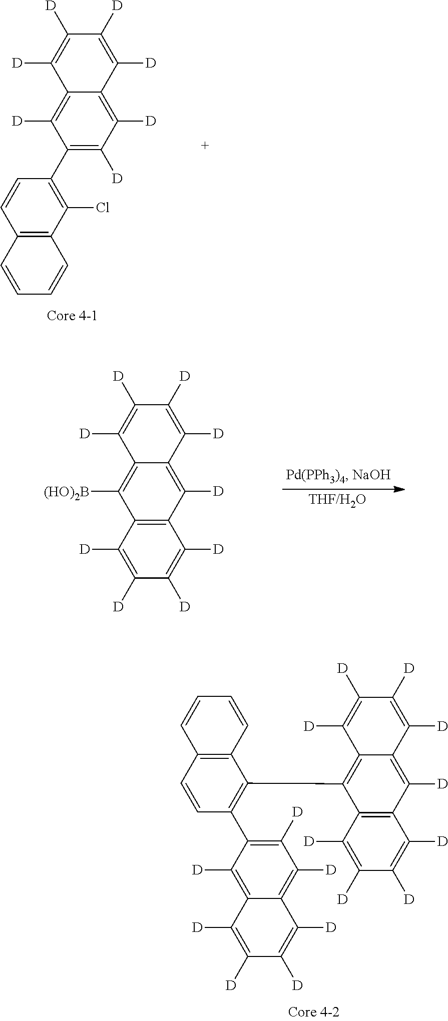 [SMILES: [2H]c1c([2H])c([2H])c2c([2H])c(-c3ccc4ccccc4c3Cl)c([2H])c([2H])c2c1[2H] ; [2H]c1c([2H])c([2H])c2c(B(O)O)c3c([2H])c([2H])c([2H])c([2H])c3c([2H])c2c1[2H] ; [2H]c1c([2H])c([2H])c2c([2H])c(-c3ccc4ccccc4c3-c3c4c([2H])c([2H])c([2H])c([2H])c4c([2H])c4c([2H])c([2H])c([2H])c([2H])c34)c([2H])c([2H])c2c1[2H]]

Core 4-1 (44 g, 150 mmol), (anthracen-9-yl-d9)boronic acid (35 g, 150 mmol), Pd(PPh₃)₄ (7 g, 6 mmol), and NaOH (18 g, 450 mmol) were added to 500 ml of THF and 250 ml of H₂O and stirred at 80° C. for 8 hours. After completion of the reaction, 200 ml of water was added thereto and stirred. After completion of the reaction, an organic layer was separated through extraction and then concentrated. The concentrated organic layer was dissolved in toluene, filtered with silica, and recrystallized with ethanol to obtain the target compound, Core 4-2 (50 g, yield 74%).

¹H-NMR: δ 7.40 (t, 1H), 7.54 (t, 2H), 7.72 (d, 1H), 8.26 (d, 1H), 7.31 (d, 1H), 8.93 (d, 1H)

<Step 3> Synthesis of Core 4

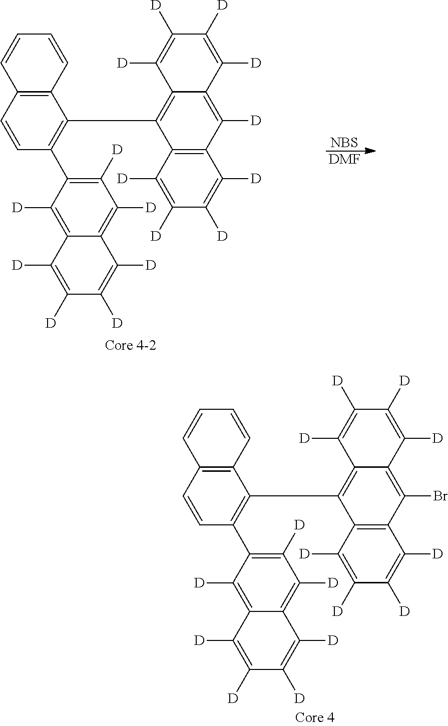

Core 4-2 (39 g, 100 mmol) and N-Bromosuccinimide (18 g, 100 mmol) were dissolved in 400 ml of dimethyl formamide, and a mixture thereof was then stirred at room temperature for 4 hours. After completion of the reaction, 400 ml of water was added thereto and a resultant solid was filtered. The filtered solid was dissolved in dichloromethane, and then filtered with silica. The organic solvent was concentrated, and then recrystallized with ethanol to obtain the target compound, Core 4 (47 g, yield 88%).

¹H-NMR: δ 7.40 (t, 1H), 7.54 (t, 2H), 7.72 (d, 1H), 8.26 (d, 1H), 7.31 (d, 1H), 8.93 (d, 1H)

### SYNTHESIS EXAMPLE

### [Synthesis Example 1] Synthesis of Compound 2

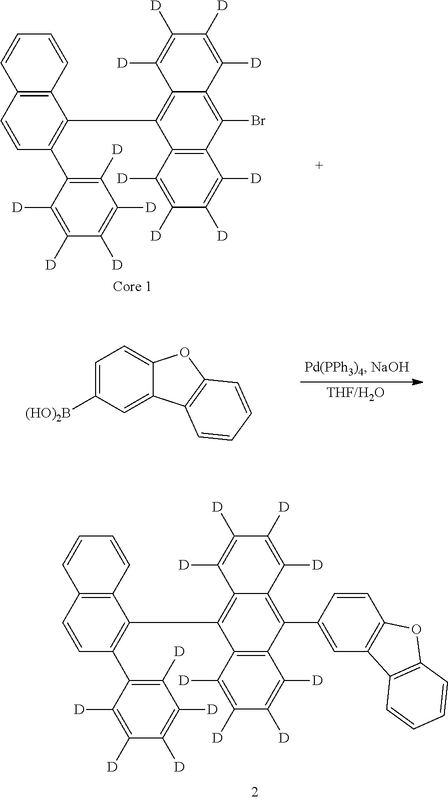 [SMILES: [2H]c1c([2H])c([2H])c(-c2ccc3ccccc3c2-c2c3c([2H])c([2H])c([2H])c([2H])c3c(Br)c3c([2H])c([2H])c([2H])c([2H])c23)c([2H])c1[2H] ; OB(O)c1ccc2oc3ccccc3c2c1 ; [2H]c1c([2H])c([2H])c(-c2ccc3ccccc3c2-c2c3c([2H])c([2H])c([2H])c([2H])c3c(-c3ccc4oc5ccccc5c4c3)c3c([2H])c([2H])c([2H])c([2H])c23)c([2H])c1[2H]]

Core 1 (4.7 g, 10.0 mmol), dibenzo[b,d]furan-2-ylboronic acid (2.1 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 2 (3.5 g, yield 63%).

[LCMS]: 559

### [Synthesis Example 2] Synthesis of Compound 3

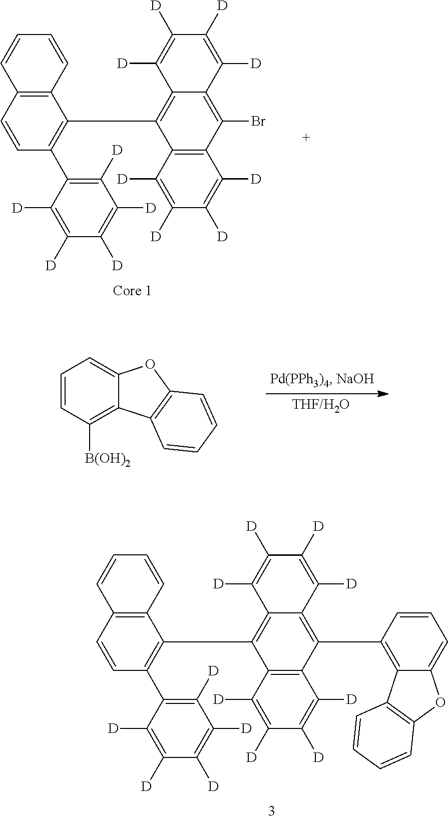 [SMILES: [2H]c1c([2H])c([2H])c(-c2ccc3ccccc3c2-c2c3c([2H])c([2H])c([2H])c([2H])c3c(Br)c3c([2H])c([2H])c([2H])c([2H])c23)c([2H])c1[2H] ; OB(O)c1cccc2oc3ccccc3c12 ; [2H]c1c([2H])c([2H])c(-c2ccc3ccccc3c2-c2c3c([2H])c([2H])c([2H])c([2H])c3c(-c3cccc4oc5ccccc5c34)c3c([2H])c([2H])c([2H])c([2H])c23)c([2H])c1[2H]]

Core 1 (4.7 g, 10.0 mmol), dibenzo[b,d]furan-1-ylboronic acid (2.1 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 3 (4.4 g, yield 78%).

[LCMS]: 559

### [Synthesis Example 3] Synthesis of Compound 6

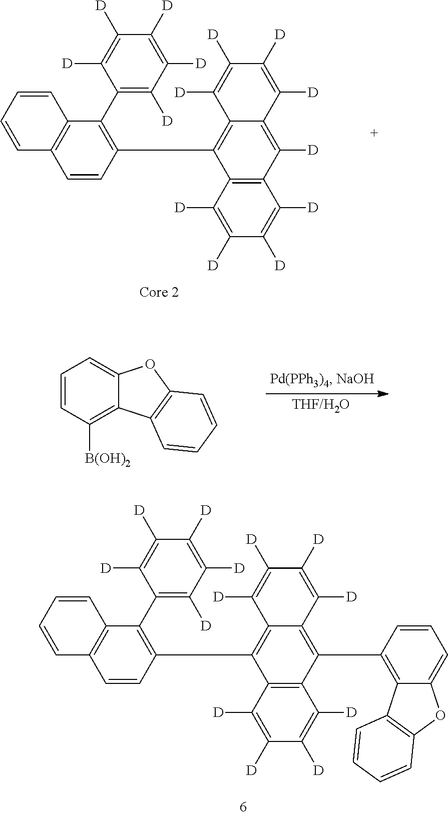 [SMILES: [2H]c1c([2H])c([2H])c(-c2c(-c3c4c([2H])c([2H])c([2H])c([2H])c4c([2H])c4c([2H])c([2H])c([2H])c([2H])c34)ccc3ccccc23)c([2H])c1[2H] ; OB(O)c1cccc2oc3ccccc3c12 ; [2H]c1c([2H])c([2H])c(-c2c(-c3c4c([2H])c([2H])c([2H])c([2H])c4c(-c4cccc5oc6ccccc6c45)c4c([2H])c([2H])c([2H])c([2H])c34)ccc3ccccc23)c([2H])c1[2H]]

Core 2 (4.7 g, 10.0 mmol), dibenzo[b,d]furan-1-ylboronic acid (2.1 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 6 (4.5 g, yield 79%).

[LCMS]: 559

### [Synthesis Example 4] Synthesis of Compound 9

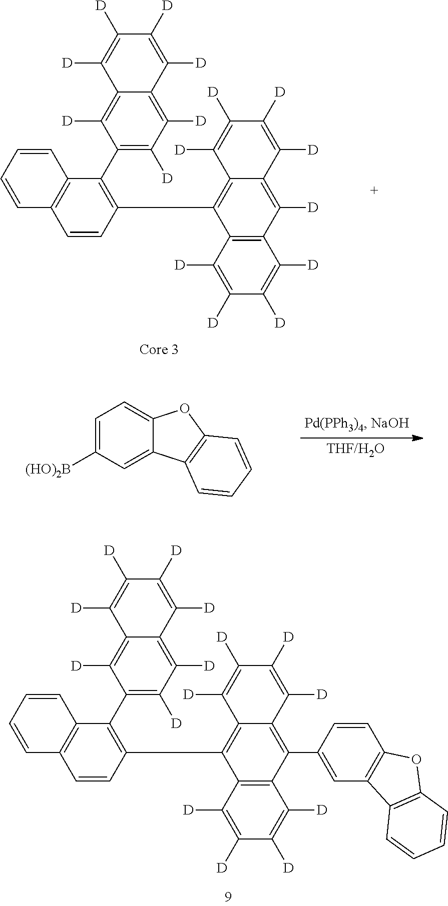 [SMILES: [2H]c1c([2H])c([2H])c2c([2H])c(-c3c(-c4c5c([2H])c([2H])c([2H])c([2H])c5c([2H])c5c([2H])c([2H])c([2H])c([2H])c45)ccc4ccccc34)c([2H])c([2H])c2c1[2H] ; OB(O)c1ccc2oc3ccccc3c2c1 ; [2H]c1c([2H])c([2H])c2c([2H])c(-c3c(-c4c5c([2H])c([2H])c([2H])c([2H])c5c(-c5ccc6oc7ccccc7c6c5)c5c([2H])c([2H])c([2H])c([2H])c45)ccc4ccccc34)c([2H])c([2H])c2c1[2H]]

Core 3 (5.2 g, 10.0 mmol), dibenzo[b,d]furan-2-ylboronic acid (2.1 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 9 (4.7 g, yield 77%).

[LCMS]: 611

### [Synthesis Example 5] Synthesis of Compound 13

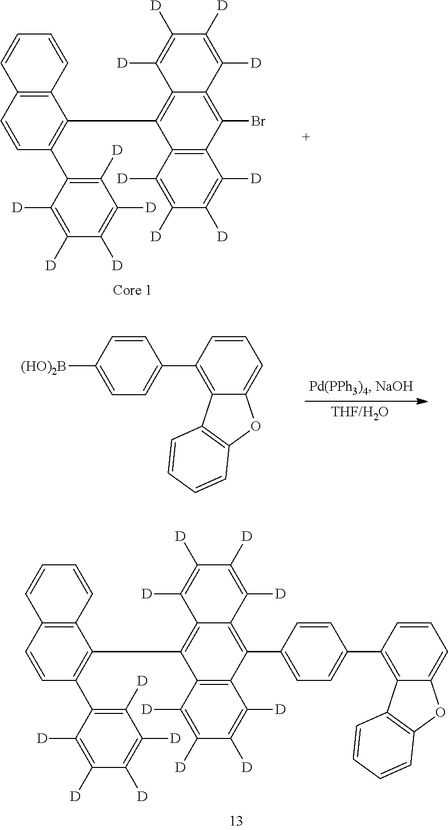

Core 1 (4.7 g, 10.0 mmol), (4-(dibenzo[b,d]furan-1-yl)phenyl)boronic acid (2.9 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 13 (5.9 g, yield 89%).

[LCMS]: 635

### [Synthesis Example 6] Synthesis of Compound 14

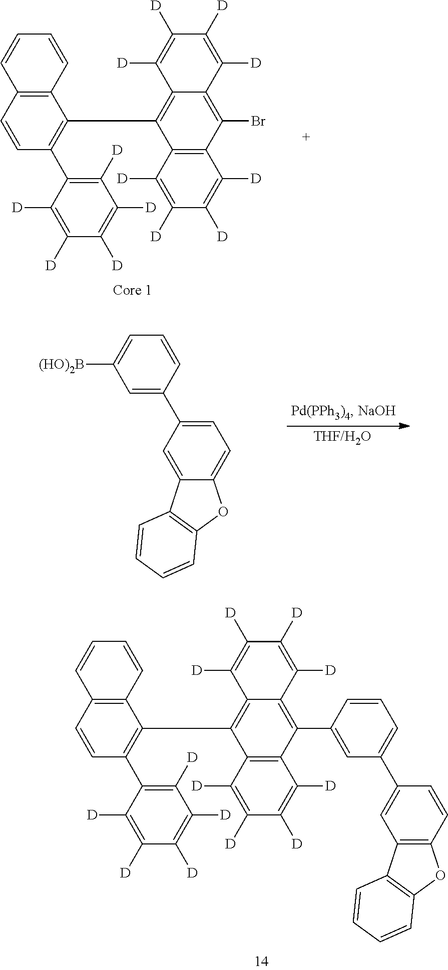 [SMILES: [2H]c1c([2H])c([2H])c(-c2ccc3ccccc3c2-c2c3c([2H])c([2H])c([2H])c([2H])c3c(Br)c3c([2H])c([2H])c([2H])c([2H])c23)c([2H])c1[2H] ; OB(O)c1cccc(-c2ccc3oc4ccccc4c3c2)c1 ; [2H]c1c([2H])c([2H])c(-c2ccc3ccccc3c2-c2c3c([2H])c([2H])c([2H])c([2H])c3c(-c3cccc(-c4ccc5oc6ccccc6c5c4)c3)c3c([2H])c([2H])c([2H])c([2H])c23)c([2H])c1[2H]]

Core 1 (4.7 g, 10.0 mmol), (3-(dibenzo[b,d]furan-2-yl)phenyl)boronic acid (2.9 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 14 (4.8 g, yield 75%).

[LCMS]: 635

### [Synthesis Example 7] Synthesis of Compound 22

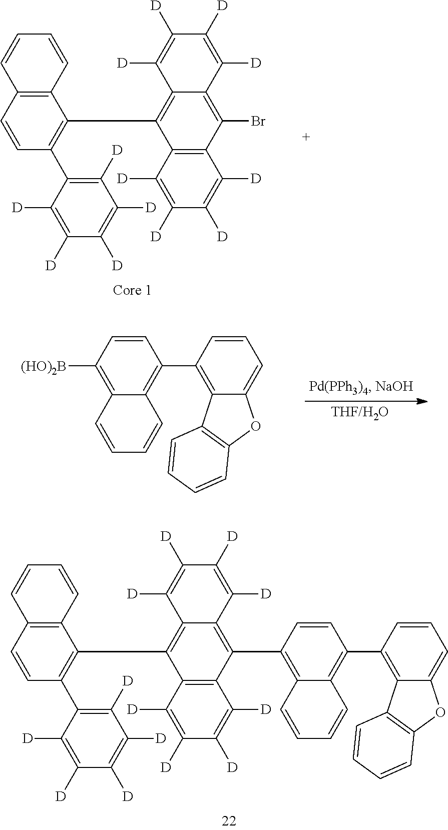 [SMILES: [2H]c1c([2H])c([2H])c(-c2ccc3ccccc3c2-c2c3c([2H])c([2H])c([2H])c([2H])c3c(Br)c3c([2H])c([2H])c([2H])c([2H])c23)c([2H])c1[2H] ; OB(O)c1ccc(-c2cccc3oc4ccccc4c23)c2ccccc12 ; [2H]c1c([2H])c([2H])c(-c2ccc3ccccc3c2-c2c3c([2H])c([2H])c([2H])c([2H])c3c(-c3ccc(-c4cccc5oc6ccccc6c45)c4ccccc34)c3c([2H])c([2H])c([2H])c([2H])c23)c([2H])c1[2H]]

Core 1 (4.7 g, 10.0 mmol), (4-(dibenzo[b,d]furan-1-yl)naphthalen-1-yl)boronic acid (3.4 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 22 (5.3 g, yield 77%).

[LCMS]: 685

### [Synthesis Example 8] Synthesis of Compound 45

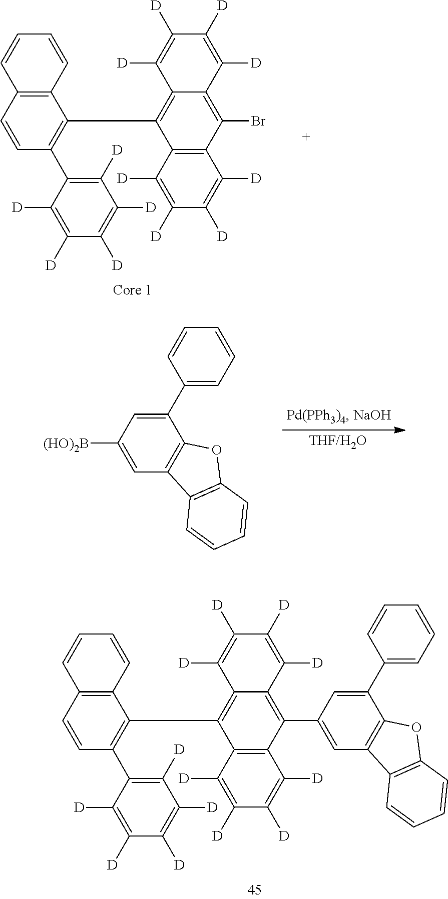 [SMILES: [2H]c1c([2H])c([2H])c(-c2ccc3ccccc3c2-c2c3c([2H])c([2H])c([2H])c([2H])c3c(Br)c3c([2H])c([2H])c([2H])c([2H])c23)c([2H])c1[2H] ; OB(O)c1cc(-c2ccccc2)c2oc3ccccc3c2c1 ; [2H]c1c([2H])c([2H])c(-c2ccc3ccccc3c2-c2c3c([2H])c([2H])c([2H])c([2H])c3c(-c3cc(-c4ccccc4)c4oc5ccccc5c4c3)c3c([2H])c([2H])c([2H])c([2H])c23)c([2H])c1[2H]]

Core 1 (4.7 g, 10.0 mmol), (4-phenyldibenzo[b,d]furan-2-yl)boronic acid (2.9 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 45 (4.9 g, yield 76%).

[LCMS]: 635

### [Synthesis Example 9] Synthesis of Compound 46

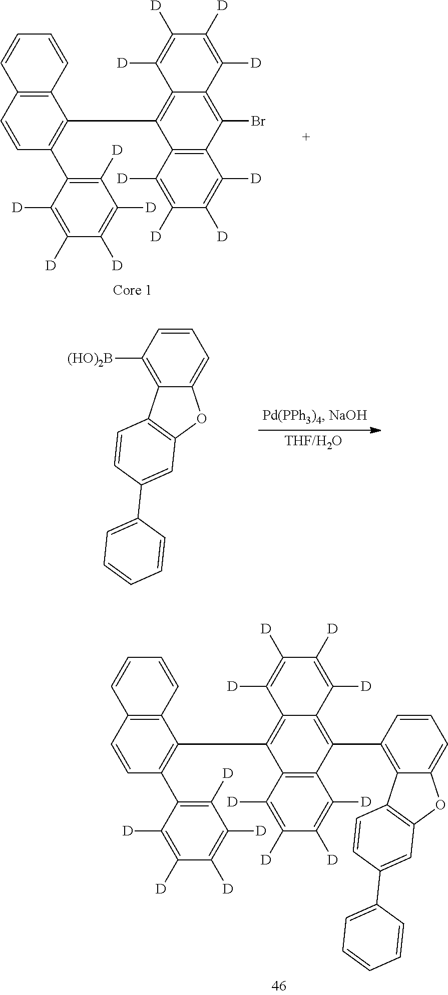 [SMILES: [2H]c1c([2H])c([2H])c(-c2ccc3ccccc3c2-c2c3c([2H])c([2H])c([2H])c([2H])c3c(Br)c3c([2H])c([2H])c([2H])c([2H])c23)c([2H])c1[2H] ; OB(O)c1cccc2oc3cc(-c4ccccc4)ccc3c12 ; [2H]c1c([2H])c([2H])c(-c2ccc3ccccc3c2-c2c3c([2H])c([2H])c([2H])c([2H])c3c(-c3cccc4oc5cc(-c6ccccc6)ccc5c34)c3c([2H])c([2H])c([2H])c([2H])c23)c([2H])c1[2H]]

Core 1 (4.7 g, 10.0 mmol), (4-(dibenzo[b,d]furan-1-yl)phenyl)boronic acid (2.9 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 46 (4.7 g, yield 73%).

[LCMS]: 635

### [Synthesis Example 10] Synthesis of Compound 51

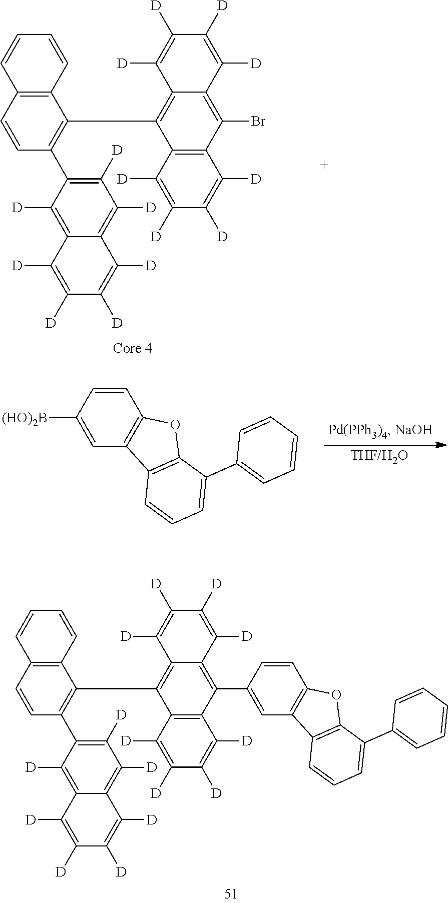 [SMILES: [2H]c1c([2H])c([2H])c2c([2H])c(-c3ccc4ccccc4c3-c3c4c([2H])c([2H])c([2H])c([2H])c4c(Br)c4c([2H])c([2H])c([2H])c([2H])c34)c([2H])c([2H])c2c1[2H] ; OB(O)c1ccc2oc3c(-c4ccccc4)cccc3c2c1 ; [2H]c1c([2H])c([2H])c2c([2H])c(-c3ccc4ccccc4c3-c3c4c([2H])c([2H])c([2H])c([2H])c4c(-c4ccc5oc6c(-c7ccccc7)cccc6c5c4)c4c([2H])c([2H])c([2H])c([2H])c34)c([2H])c([2H])c2c1[2H]]

Core 4 (5.2 g, 10.0 mmol), (6-phenyldibenzo[b,d]furan-2-yl)boronic acid (2.9 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added thereto and stirred. A resultant solid was filtered. After filtering, the solid was dissolved in toluene, followed by silica filtering, and then recrystallized with toluene to obtain the target compound, Compound 51 (5.9 g, yield 85%).

[LCMS]: 687

### [Examples 1 to 10] Manufacturing of Blue Organic EL Elements

The compound synthesized in Synthesis Examples 1 to 10 were subjected to high-purity sublimation purification in a conventionally known method, and then blue organic EL elements were manufactured according to the following procedure.

First, a glass substrate thin-film-coated with indium tin oxide (ITO) to a thickness of 1500 Å was washed with distilled water ultrasonically. After washing with distilled water was completed, the glass substrate was ultrasonically cleaned with a solvent, such as isopropyl alcohol, acetone and methanol, dried, transferred to a UV OZONE cleaner (Power sonic 405, Hwasin Tech) cleaned for 5 minutes using UV, and then transferred to a vacuum evaporator.

On the ITO transparent electrode prepared as above, DS-205 (Doosan Electronics Co., Ltd., 80 nm)/NPB (15 nm)/respective compounds+ADN+5% DS-405 (Doosan Electronics Co., Ltd., 300 nm)/BCP (10 nm)/Alq₃ (30 nm)/LiF (1 nm)/Al (200 nm) were stacked in order to manufacture organic EL elements.

For example, structures of NPB, ADN, BCP, Compounds A-1 to A-4 as used herein in Examples (Ex.) and Comparative Examples (Comp. Ex.) are as follows.

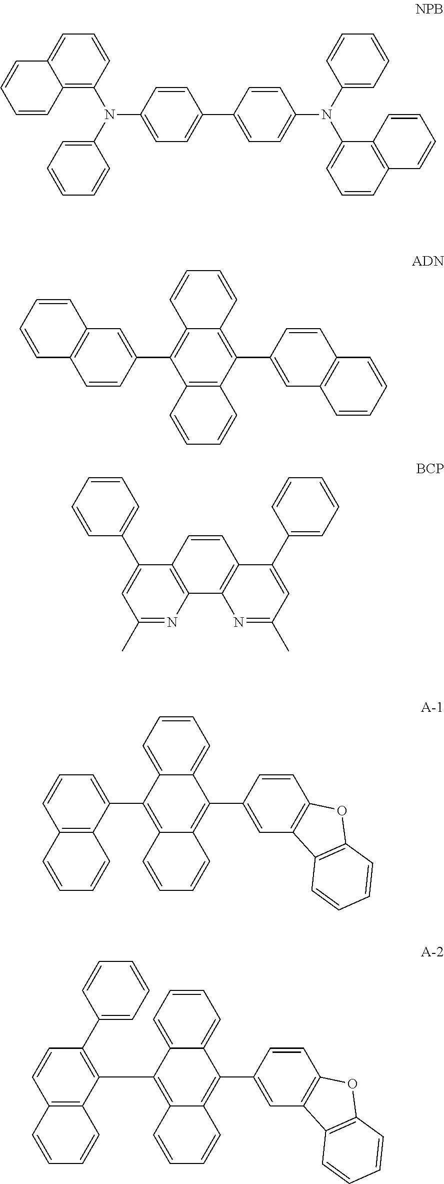

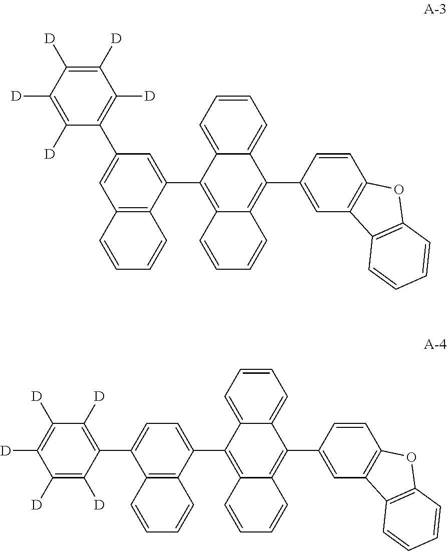 [SMILES: [2H]c1c([2H])c([2H])c(-c2cc(-c3c4ccccc4c(-c4ccc5oc6ccccc6c5c4)c4ccccc34)c3ccccc3c2)c([2H])c1[2H] ; [2H]c1c([2H])c([2H])c(-c2ccc(-c3c4ccccc4c(-c4ccc5oc6ccccc6c5c4)c4ccccc34)c3ccccc23)c([2H])c1[2H]]

### [Comparative Example 1] Manufacturing of Blue Organic EL Element

A blue organic EL element of Comparative Example 1 was manufactured in the same manner as in Example 1, except that Compound A-1 was used instead of Compound 2 used in Example 1.

### [Comparative Example 2] Manufacturing of Blue Organic EL Element

A blue organic EL element of Comparative Example 2 was manufactured in the same manner as in Example 1, except that Compound A-2 was used instead of Compound 2 used in Example 1.

### [Comparative Example 3] Manufacturing of Blue Organic EL Element

A blue organic EL element of Comparative Example 3 was manufactured in the same manner as in Example 1, except that Compound A-3 was used instead of Compound 2 used in Example 1.

### [Comparative Example 4] Fabrication of Blue Organic EL Element

A blue organic EL element of Comparative Example 4 was manufactured in the same manner as in Example 1, except that Compound A-4 was used instead of Compound 2 used in Example 1.

### Evaluation Example

For each of the blue organic EL elements manufactured in Examples 1 to 10 and Comparative Examples 1 to 4, a driving voltage, a current efficiency and a lifespan at a current density of 10 mA/cm² were measured, and the results are shown in Table 1 below.

TABLE 1

| | Light | Driving | Current | Lifespan |
|---|---|---|---|---|
| | emitting | voltage | efficiency | T₉₅ |
| Sample | layer | (V) | (cd/A) | (hr) |
| Ex. 1 | 2 | 3.6 | 9.2 | 300 |
| Ex. 2 | 3 | 3.4 | 9.6 | 330 |
| Ex. 3 | 6 | 3.5 | 9.2 | 310 |
| Ex. 4 | 9 | 3.7 | 8.9 | 310 |
| Ex. 5 | 13 | 3.2 | 9.3 | 305 |
| Ex. 6 | 14 | 3.3 | 9.1 | 290 |
| Ex. 7 | 22 | 3.7 | 9.0 | 270 |
| Ex. 8 | 45 | 3.8 | 9.0 | 300 |
| Ex. 9 | 46 | 3.4 | 9.7 | 330 |
| Ex. 10 | 51 | 3.5 | 8.9 | 310 |
| Comp. Ex. 1 | A-1 | 5.0 | 6.4 | 100 |
| Comp. Ex. 2 | A-2 | 4.3 | 7.0 | 100 |
| Comp. Ex. 3 | A-3 | 4.7 | 7.2 | 150 |
| Comp. Ex. 4 | A-4 | 4.8 | 7.0 | 180 |

As shown in Table 1, it was appreciated that the blue organic EL elements of Examples 1 to 10 that used the compound represented by Chemical Formula 1 according to the present invention as a light emitting layer material are superior in terms of the driving voltage, current efficiency and lifespan compared to the blue organic EL elements of Comparative Examples 1 and 2 in which deuterium is not contained in the molecule; and Comparative Examples 3 and 4 which uses a light emitting layer material in which anthracene and a naphthyl group are included, but the naphthyl group is substituted in meta- and para-positions. In particular, it was confirmed that the lifespan characteristics of the element was significantly improved by about 2 to 3 times.

1-16. (canceled)

17. A compound of the following Chemical Formula 1: 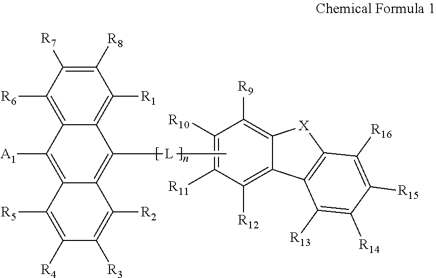
wherein in Chemical Formula 1,
R₁ to R₈ are the same as or different from each other, each independently being hydrogen or deuterium, provided that at least one of R₁ to R₈ is deuterium,
X is O, S or CRₐR_{b},
Rₐ and R_{b} are the same as or different from each other, each independently being a C₁ to C₄₀ alkyl group or a C₆ to C₆₀ aryl group, or Rₐ and R_{b} being bonded to each other to form a condensed ring,
R₉ to R₁₆ are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group,
L is a single bond or is selected from: a C₆ to C₁₈ arylene group and a heteroarylene group having 5 to 18 nuclear atoms,
n is an integer in a range from 0 to 2,
A₁ is a substituent represented by the following Chemical Formula 2, 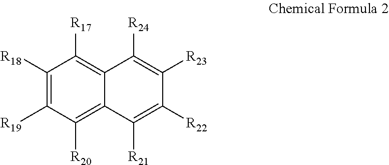
in Chemical Formula 2,
one of R₁₇ to R₂₄ is bonded to Chemical Formula 1, and another of R₁₇ to R₂₄ is bonded to an aryl group A₂ having at least one deuterium,
Chemical Formula 1 and the aryl group A₂ having at least one deuterium are bonded in an ortho position,
the others of R₁₇ to R₂₄ that are not bonded to Chemical Formula 1 and the aryl group having at least one deuterium are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group, and
the arylene group and the heteroarylene group of L and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the phosphine group, the phosphine oxide group, and the arylamine group of R₉ to R₁₄, Rₐ, R_{b}, and the others of R₁₇ to R₂₄ that are not bonded to Chemical Formula 1 and the aryl group having at least one deuterium are each independently substitutable with one or more kinds of substituents selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and when the substituents are plural in number, the substituents are the same as or different from each other.

18. The compound of claim 17, wherein one of R₁₇ and R₁₈ in Chemical Formula 2 is bonded to Chemical Formula 1, and the other of R₁₇ and R₁₈ is bonded to the aryl group A₂ having at least one deuterium.

19. The compound of claim 17, wherein the aryl group A₂ having at least one deuterium is represented by any one of Chemical Formulas 3a to 3c: 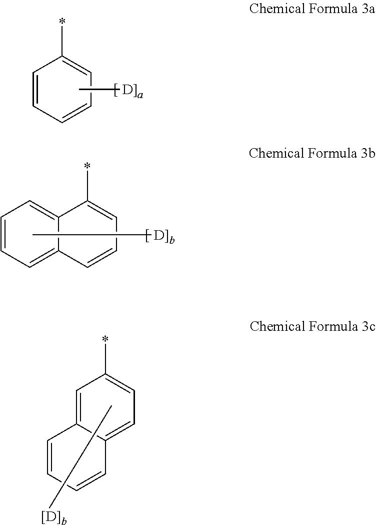
wherein in Chemical Formulas 3a to 3c,
* indicates a site where a bond with Chemical Formula 2 is made,
a is an integer in a range from 0 to 5, and
b is an integer in a range from 0 to 7.

20. The compound of claim 19, wherein the aryl group A₂ having at least one deuterium is selected from the following structural formulas: 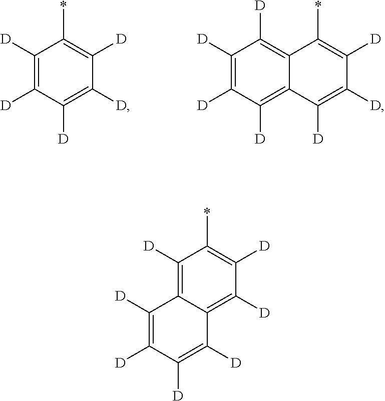
wherein in the above structural Formulas,
* indicates a site where a bond with Chemical Formula 2 is made.

21. The compound of claim 17, wherein Chemical Formula 2 is selected from substituents of the following structural formulas:

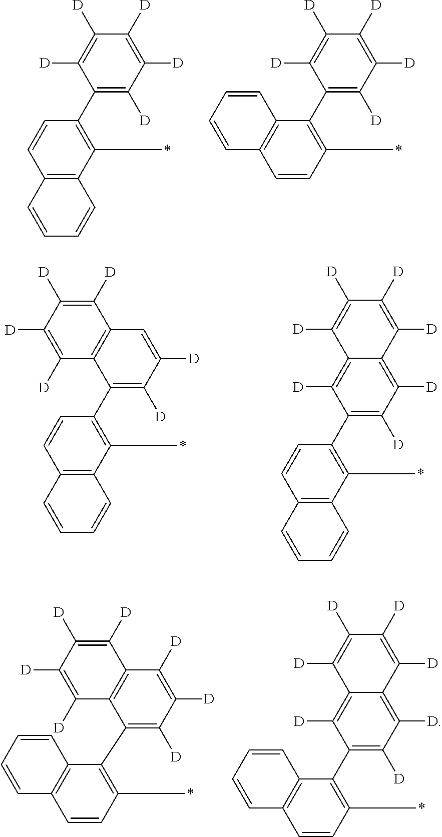

22. The compound of claim 17, wherein L is a single bond, or is selected from substituents of the following structural formulas: 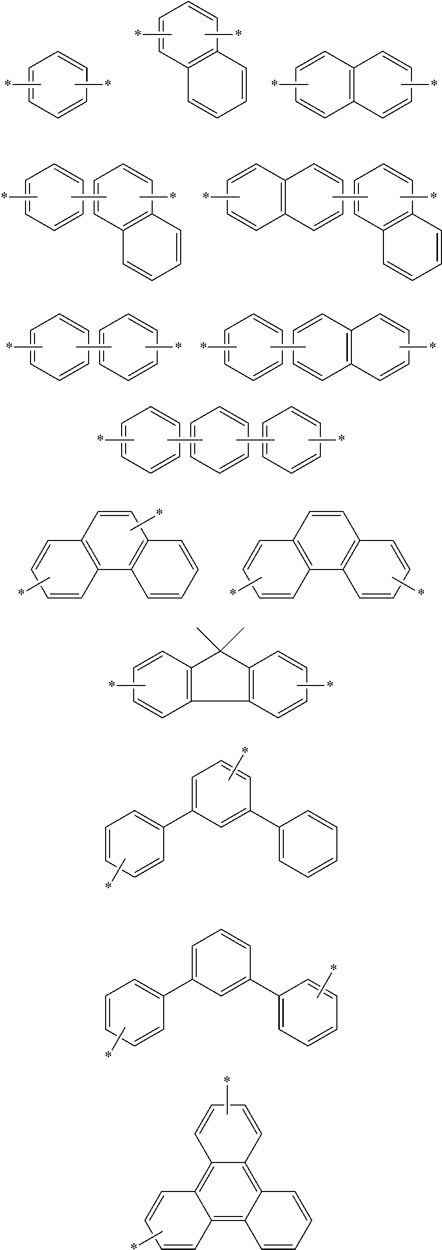
wherein in the above structural formulas,
* indicates a site where a bond with Chemical Formula 1 is made.

23. The compound of claim 17, wherein R₉ to R₁₄ are the same as or different from each other, each independently being selected from: hydrogen, deuterium, a C₁ to C₄₀ alkyl group, and a C₆ to C₆₀ aryl group.

24. The compound of claim 17, wherein the compound of Chemical Formula 1 is represented by any one of Chemical Formulas 4 to 9 below: 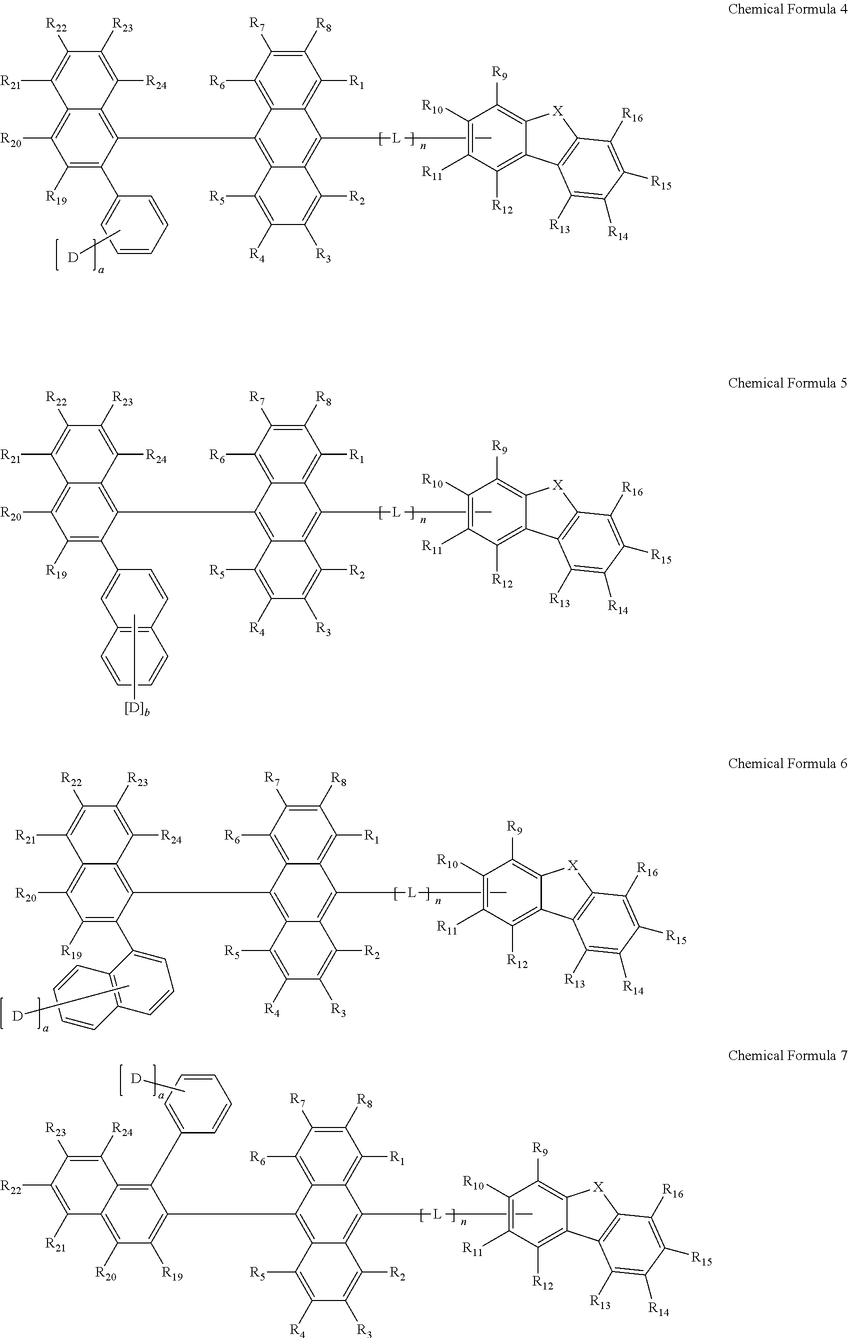 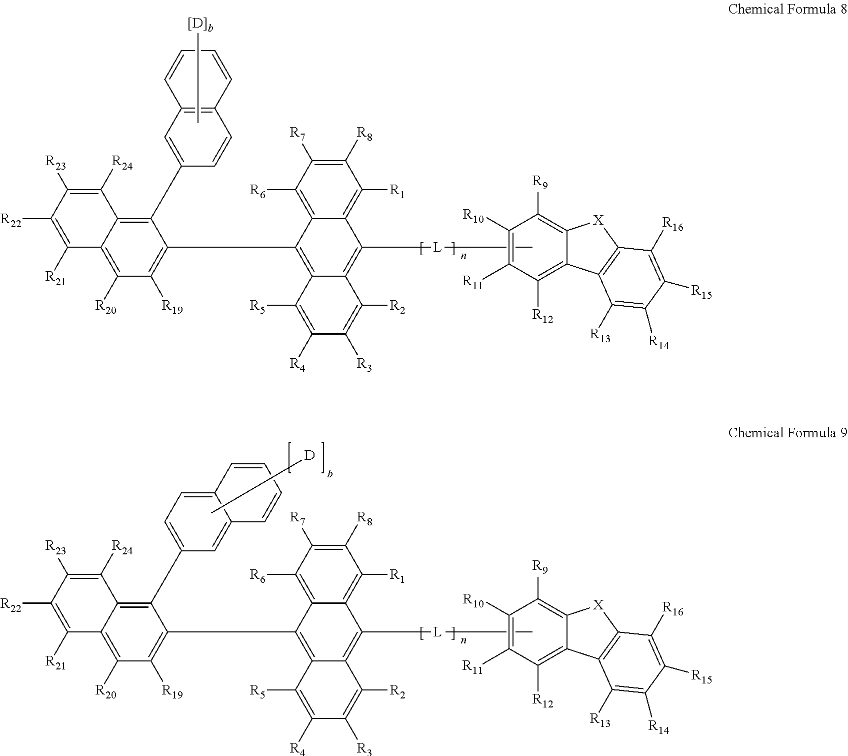
wherein in Chemical Formulas 4 to 9,
X, L, R₁ to R₁₆, R₁₉ to R₂₄, n, a and b are each as defined in claim 17.

25. The compound of claim 17, wherein the compound of Chemical Formula 1 is represented by any one of Chemical Formulas 10 to 13 below: 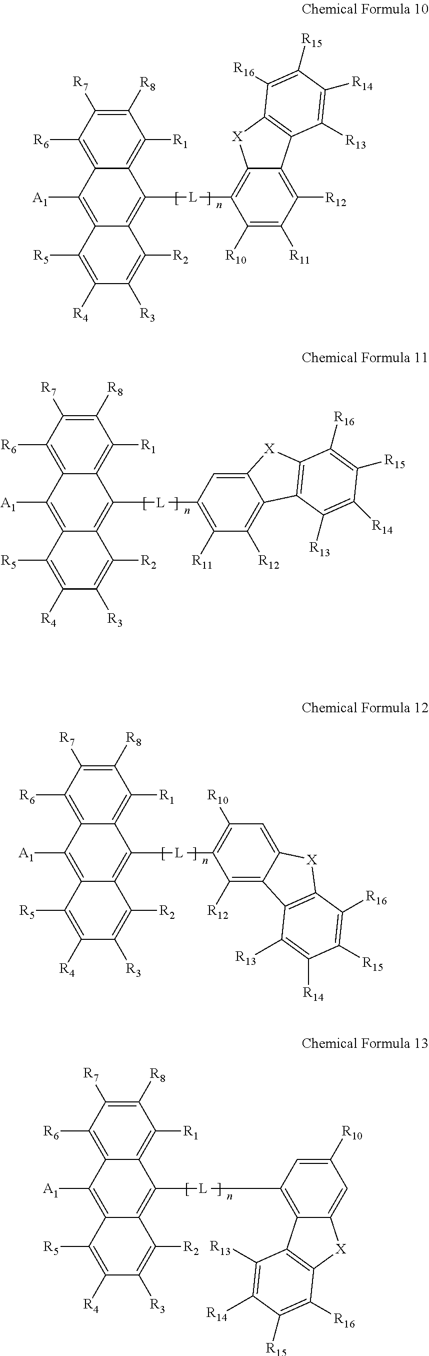
wherein in Chemical Formulas 10 to 13,
A₁, X, L, R₁ to R₁₆, R₁₉ to R₂₄, n, a and b are each as defined in claim 17.

26. The compound of claim 17, wherein the compound of Chemical Formula 1 is represented by any one of Chemical Formulas 14 to 16 below: 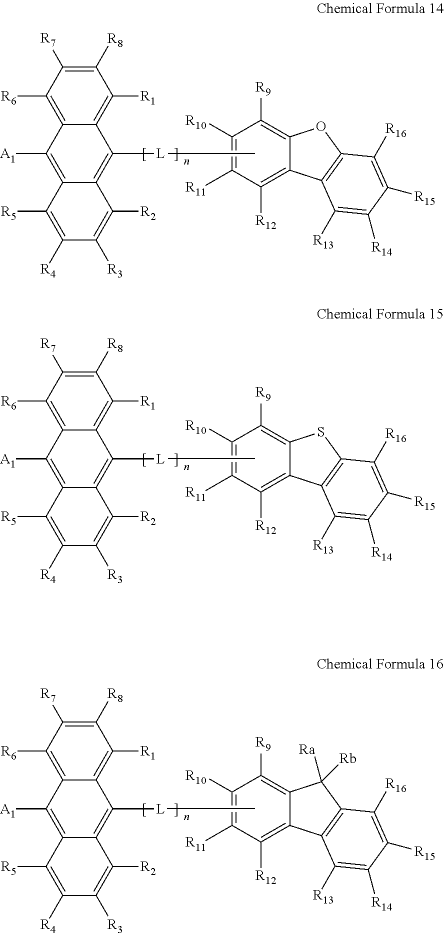
wherein in Chemical Formulas 14 to 16,
A₁, X, L, R₁ to R₁₆, R₁₉ to R₂₄, Rₐ, R_{b}, and n are each as defined in claim 17.

27. The compound of claim 17, wherein the compound represented by Chemical Formula 1 is represented by Chemical Formula 17 or Chemical Formula 18 below: 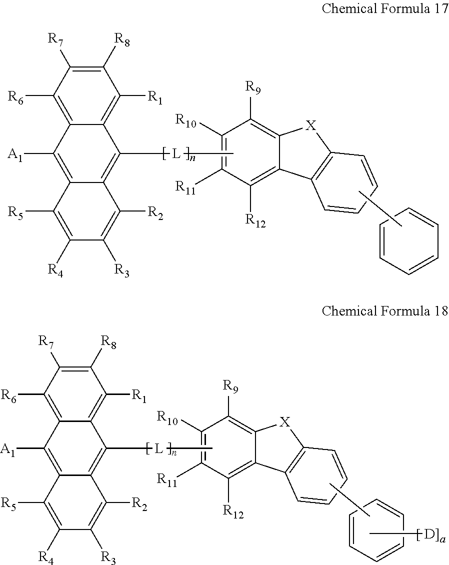
wherein in Chemical Formulas 17 to 18,
A₁, X, L, R₁ to R₁₂, and n are each as defined in claim 17.

28. The compound of claim 17, wherein the compound represented by Chemical Formula 1 is selected from compounds represented by the following Chemical Formulas:

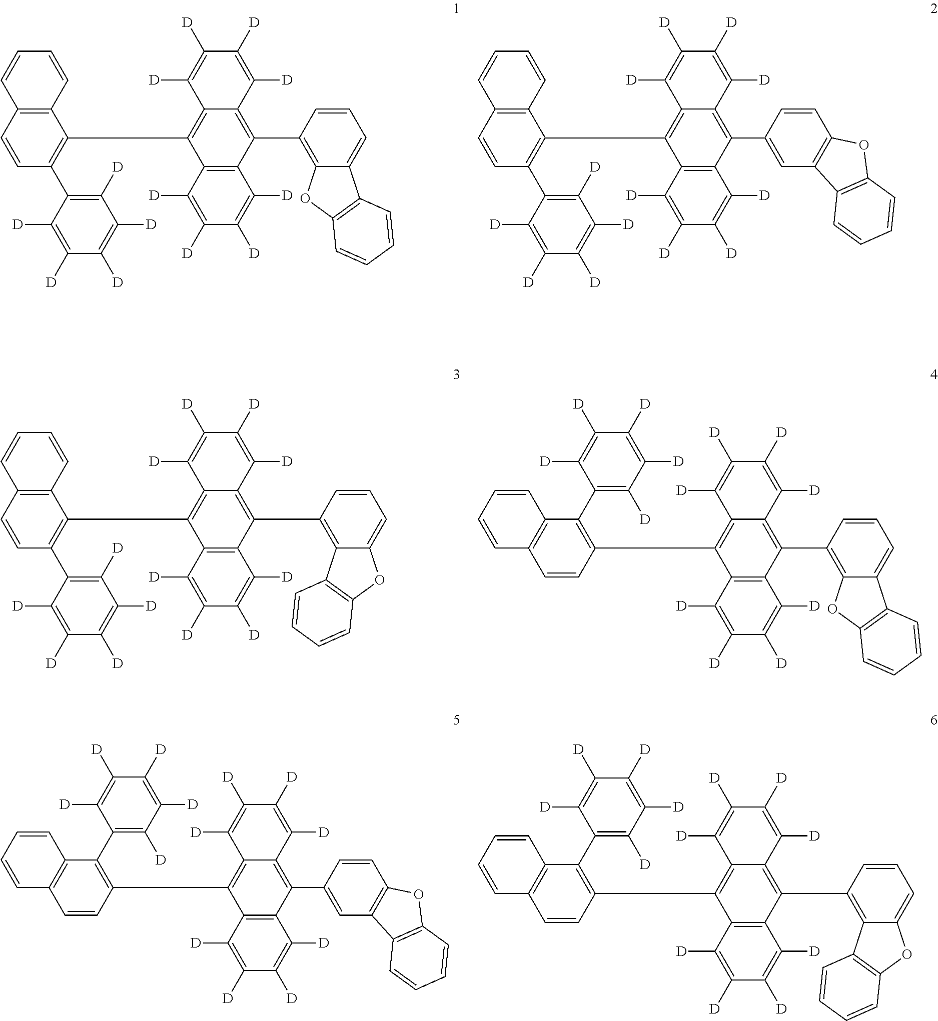

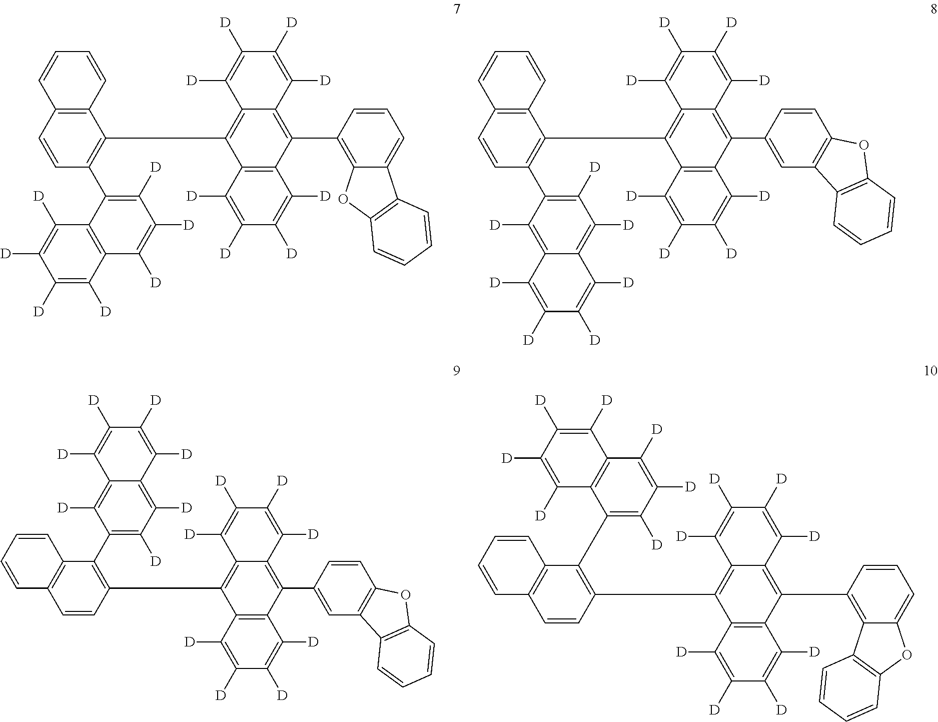

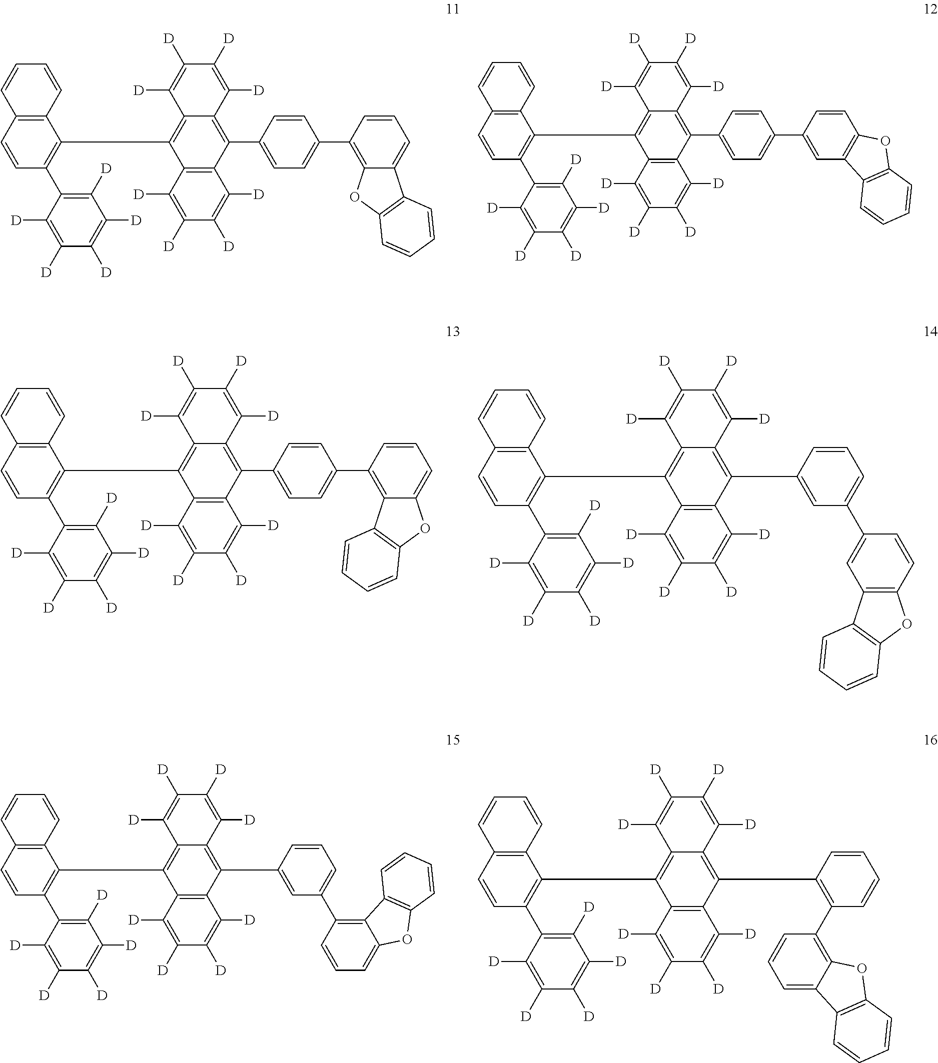

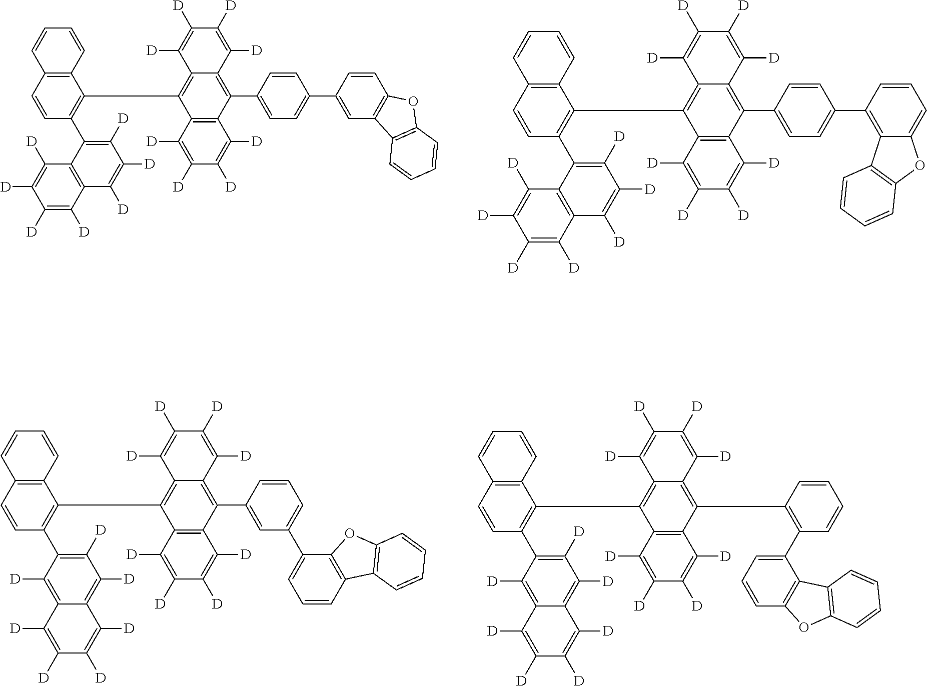

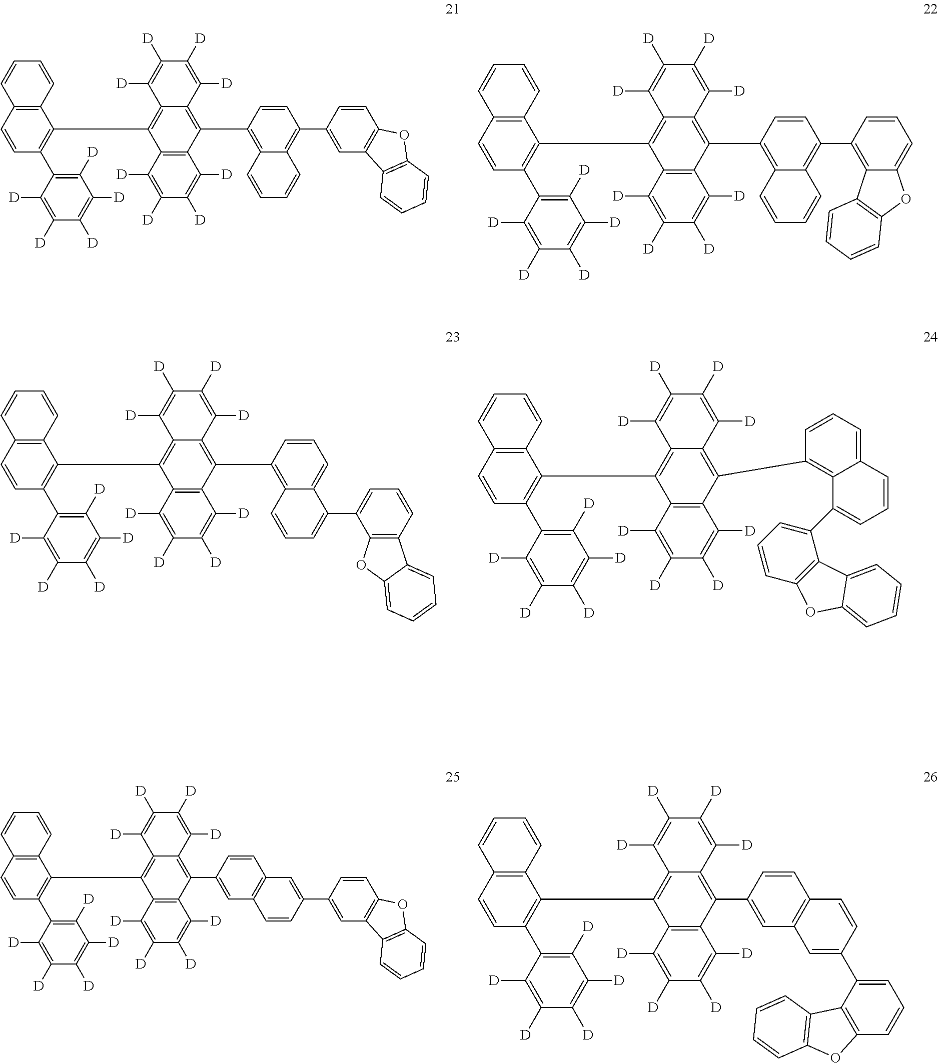

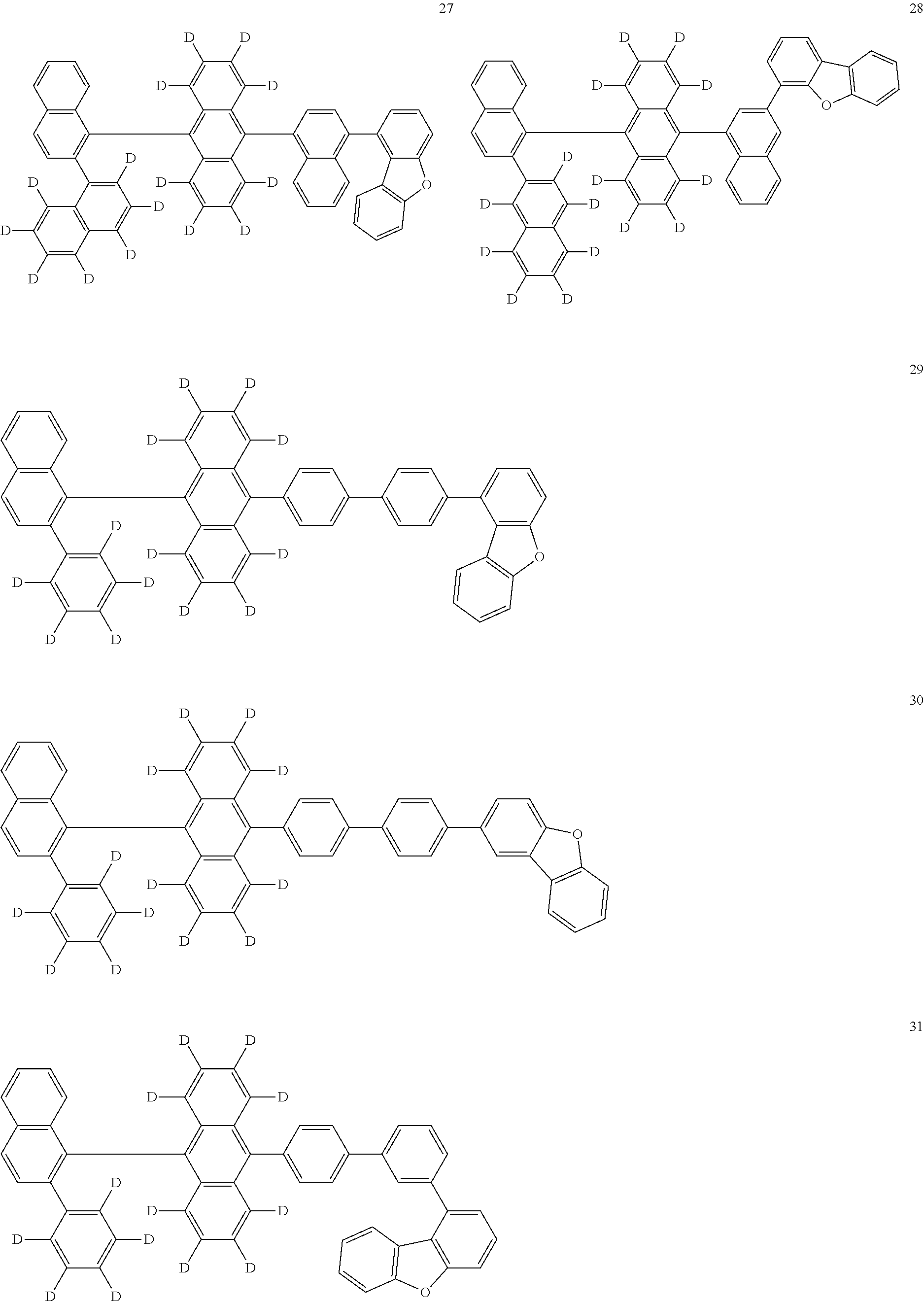

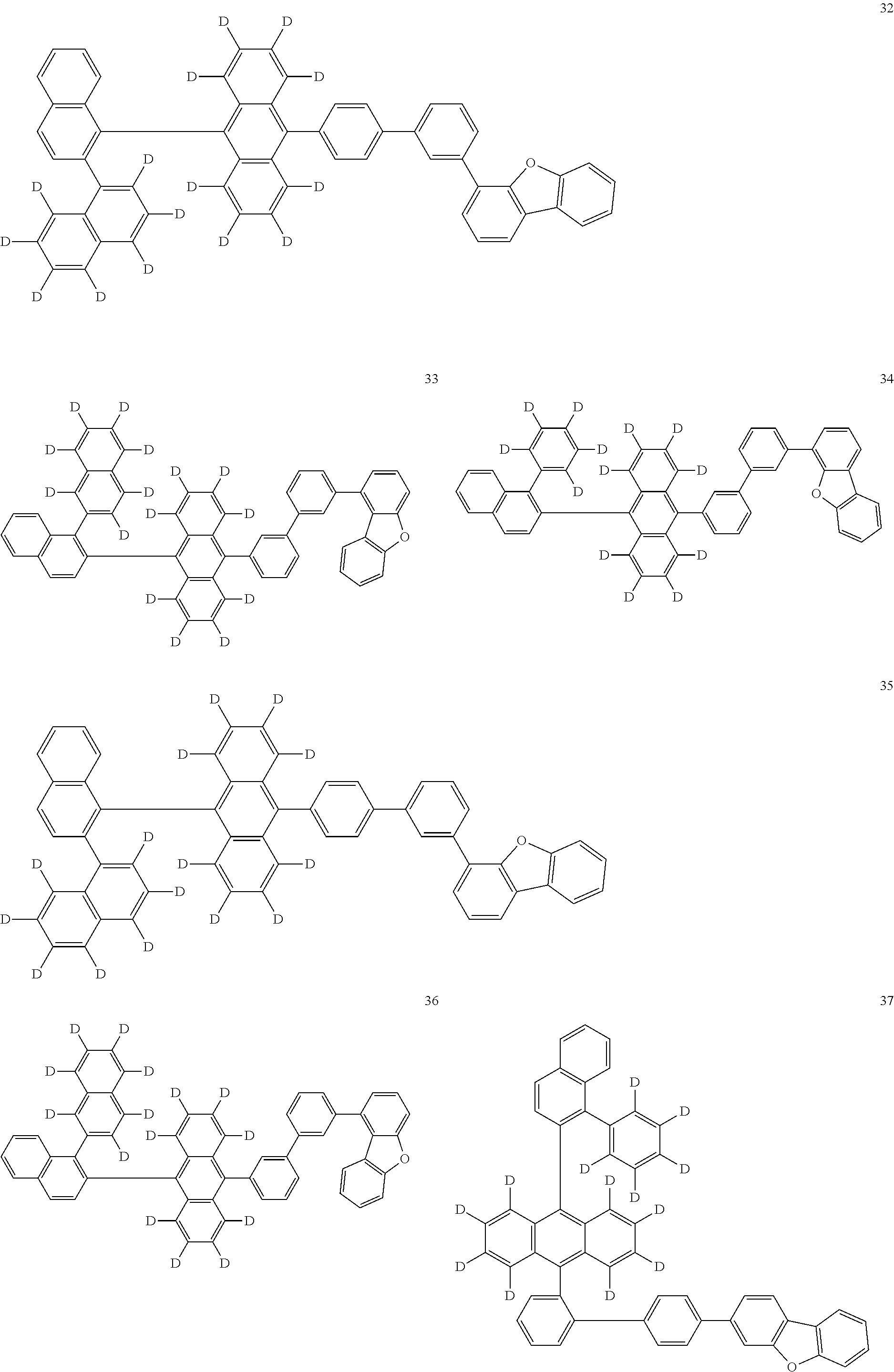

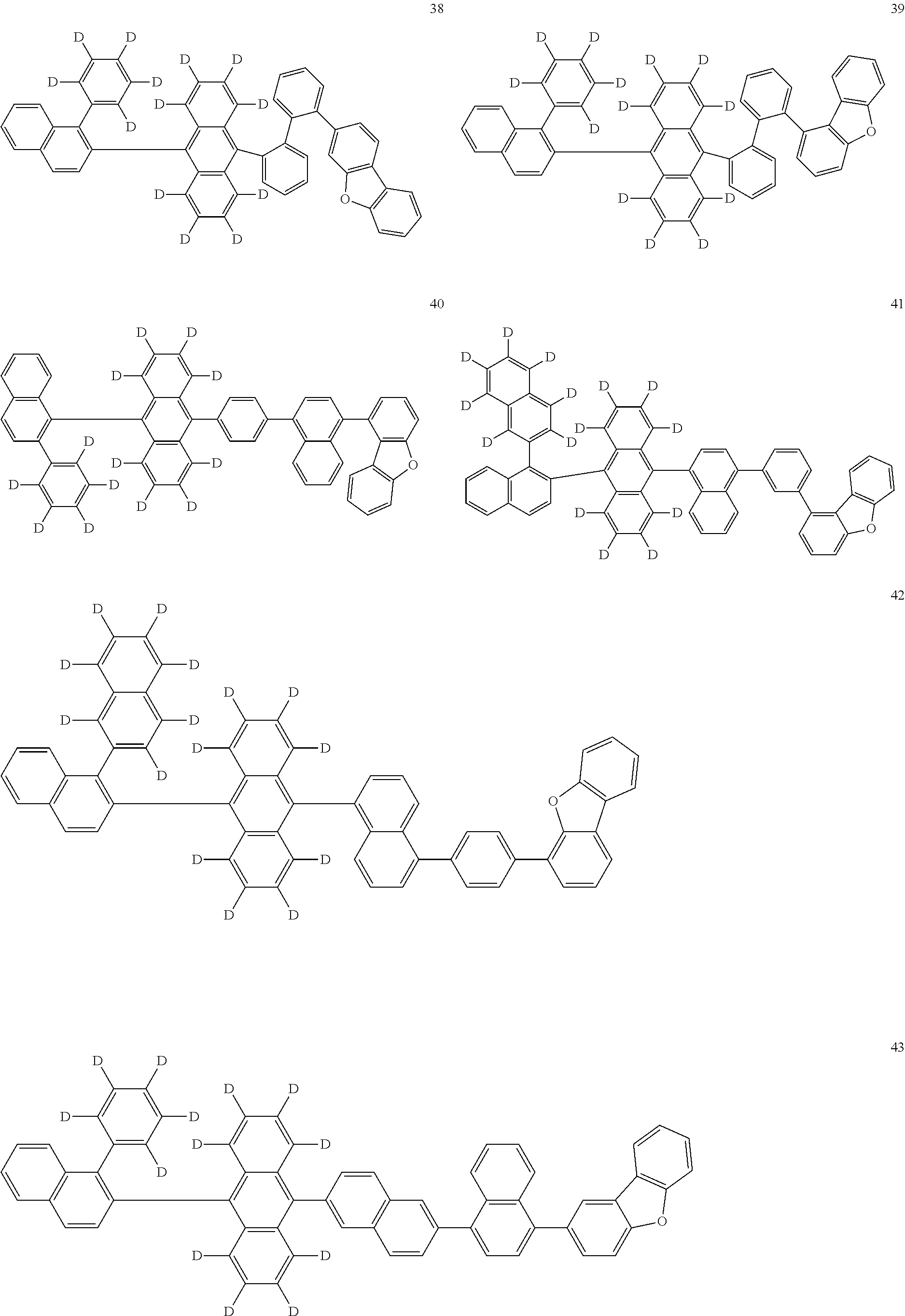

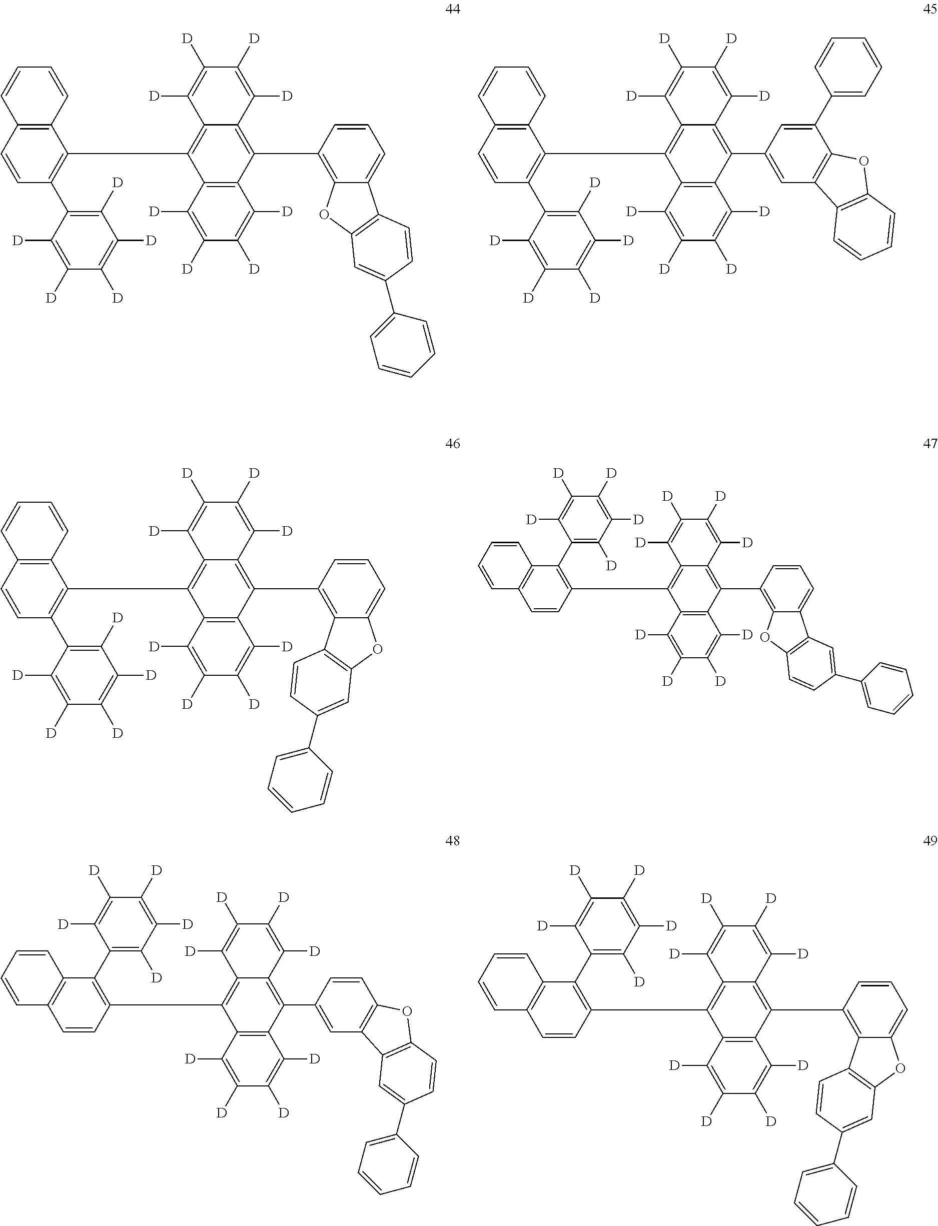

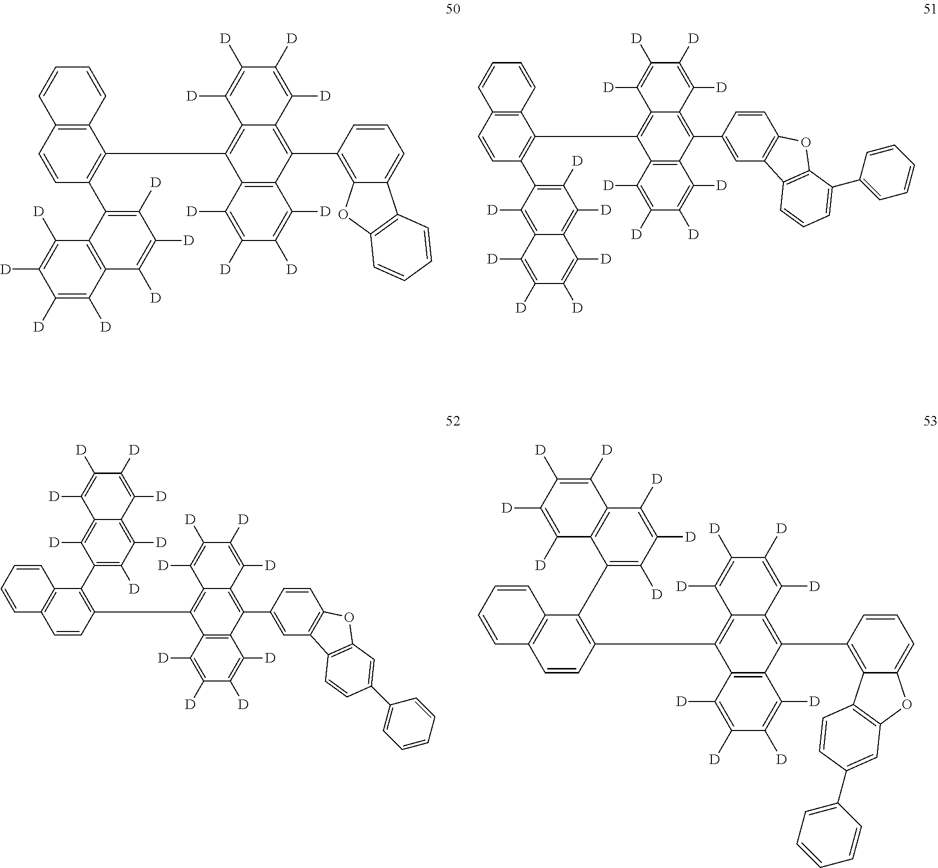

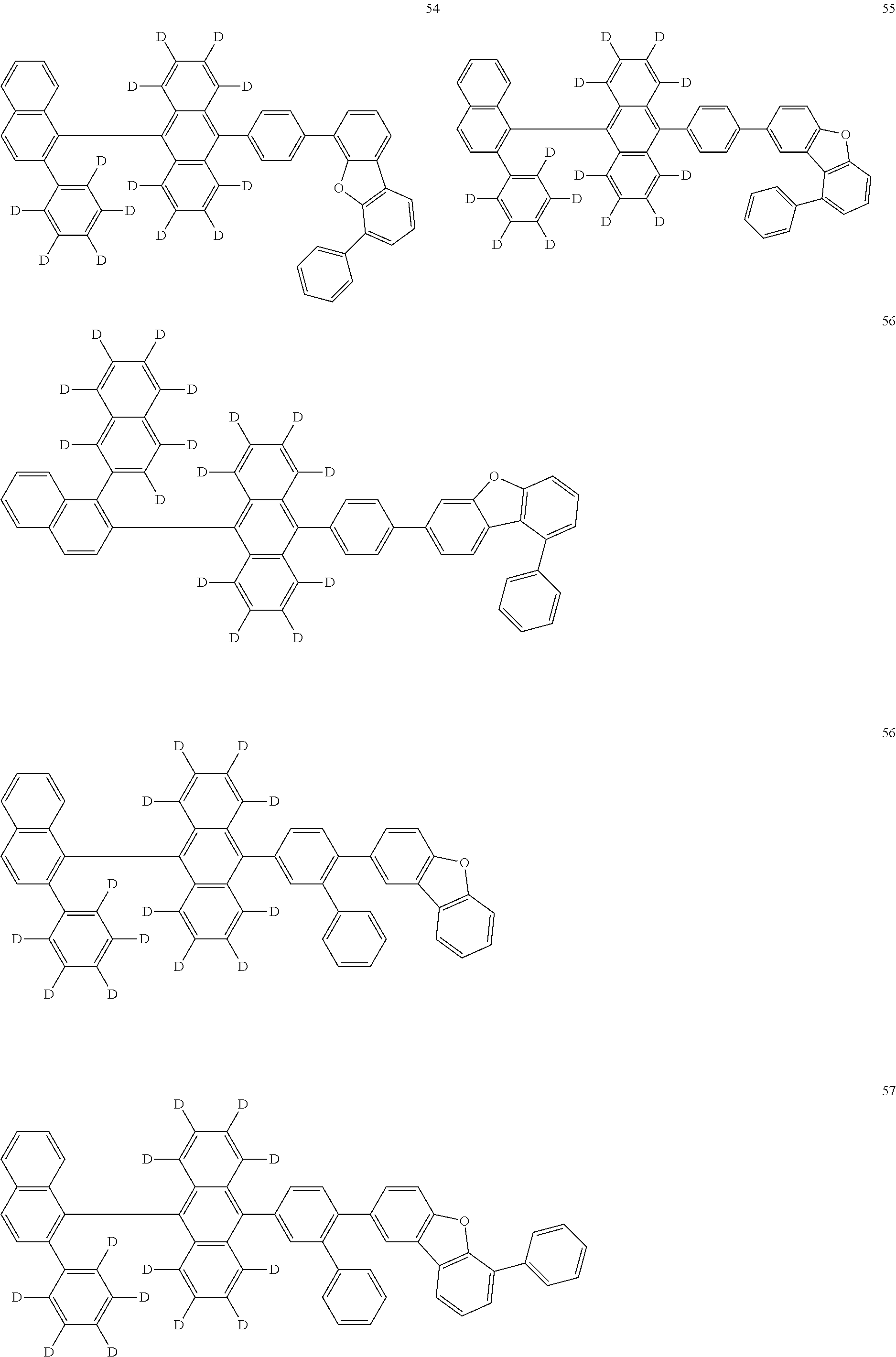

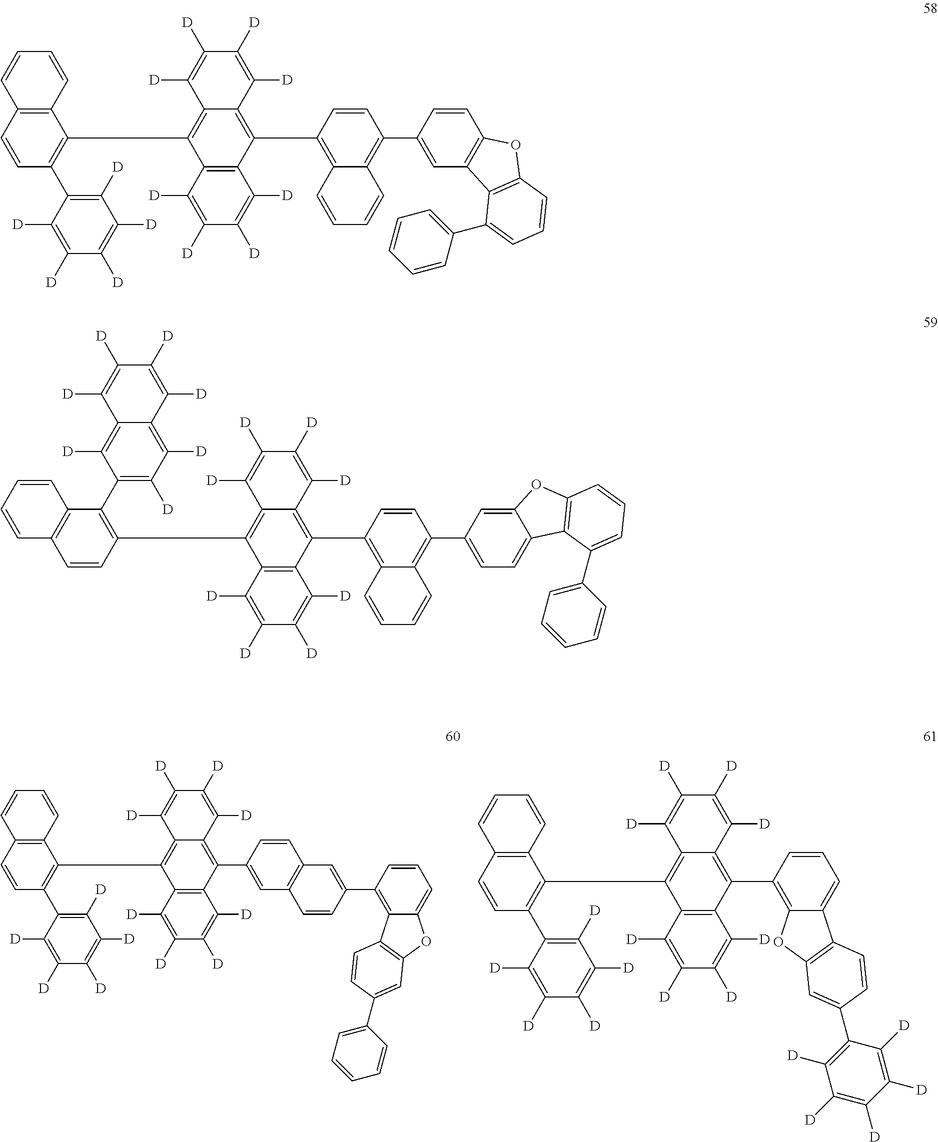

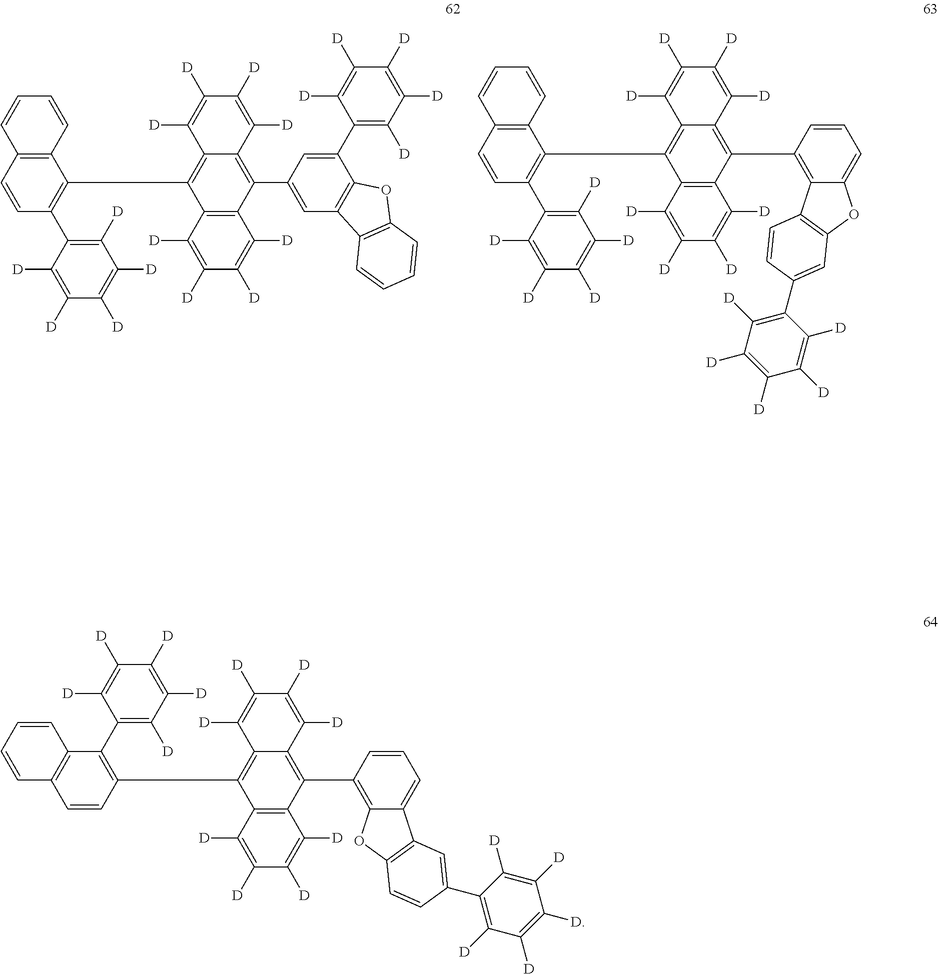

29. The compound of claim 17, wherein the compound of Chemical Formula 1 is a fluorescent host material.

30. An organic electroluminescent element comprising: an anode, a cathode, and one or more organic layers disposed between the anode and the cathode, wherein at least one of the one or more organic layers comprises the compound of Chemical Formula 1 according to claim 17.

31. The organic electroluminescent element of claim 30, wherein the compound of Chemical Formula 1 is represented by any one of Chemical Formulas 4 to 9 below: 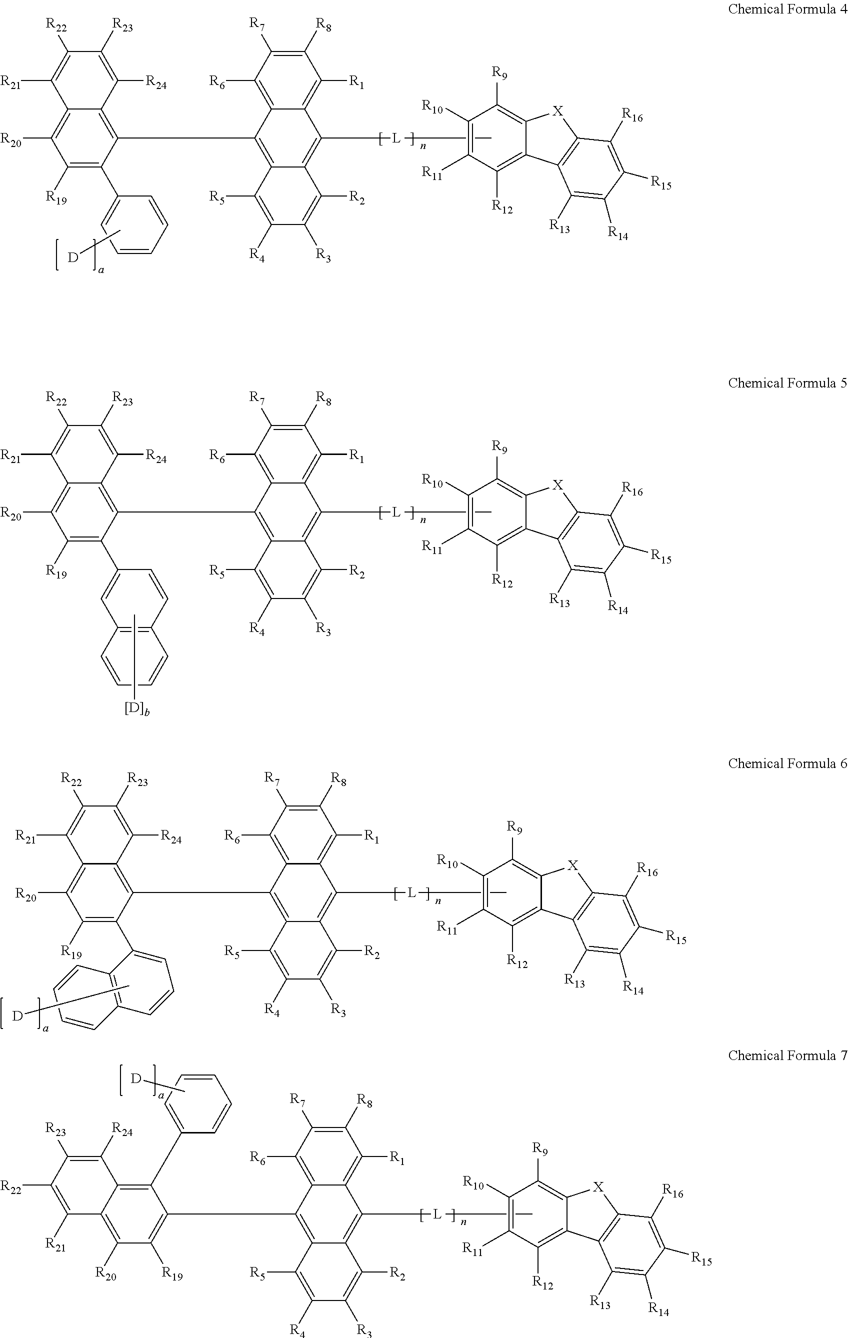 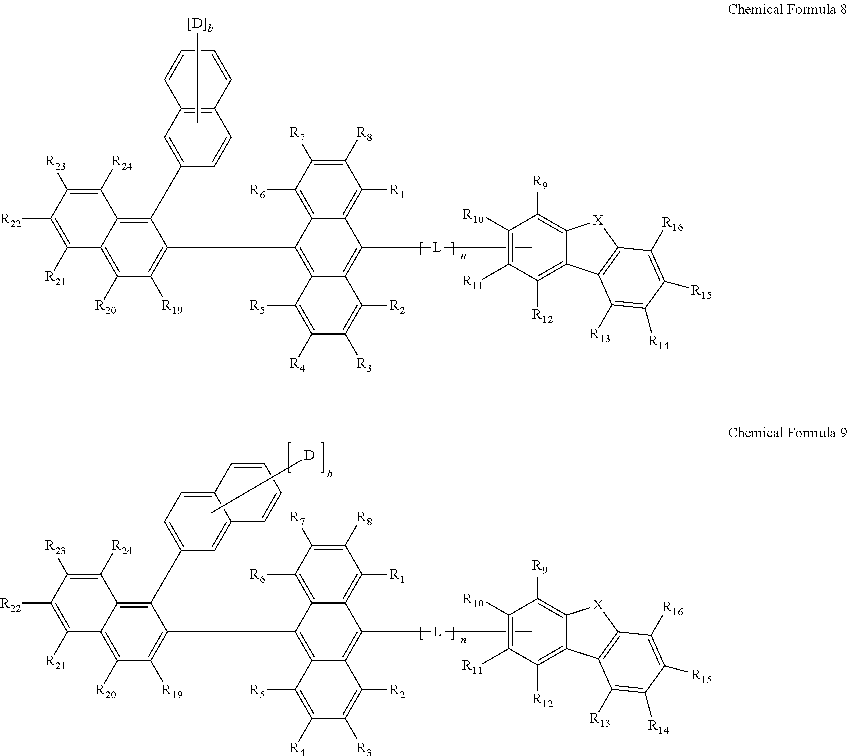
wherein in Chemical Formulas 4 to 9,
R₁ to R₈ are the same as or different from each other, each independently being hydrogen or deuterium, provided that at least one of R₁ to R₈ is deuterium,
X is O, S or CRₐR_{b},
Rₐ and R_{b} are the same as or different from each other, each independently being a C₁ to C₄₀ alkyl group or a C₆ to C₆₀ aryl group, or Rₐ and R_{b} being bonded to each other to form a condensed ring,
R₉ to R₁₆ are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group,
L is a single bond or is selected from: a C₆ to C₁₈ arylene group and a heteroarylene group having 5 to 18 nuclear atoms,
n is an integer in a range from 0 to 2,
A₁ is a substituent represented by the following Chemical Formula 2, 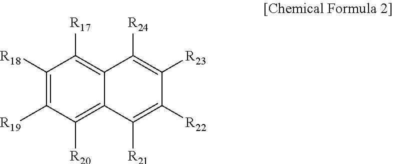
in Chemical Formula 2,
one of R₁₇ to R₂₄ is bonded to Chemical Formula 1, and another of R₁₇ to R₂₄ is bonded to an aryl group A₂ having at least one deuterium,
Chemical Formula 1 and the aryl group A₂ having at least one deuterium are bonded in an ortho position,
the others of R₁₇ to R₂₄ that are not bonded to Chemical Formula 1 and the aryl group having at least one deuterium are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group, and
the arylene group and the heteroarylene group of L and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the phosphine group, the phosphine oxide group, and the arylamine group of R₉ to R₁₄, Rₐ, R_{b}, and the others of R₁₇ to R₂₄ that are not bonded to Chemical Formula 1 and the aryl group having at least one deuterium are each independently substitutable with one or more kinds of substituents selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and when the substituents are plural in number, the substituents are the same as or different from each other.

32. The organic electroluminescent element of claim 30, wherein the compound of Chemical Formula 1 is represented by any one of Chemical Formulas 10 to 13 below: 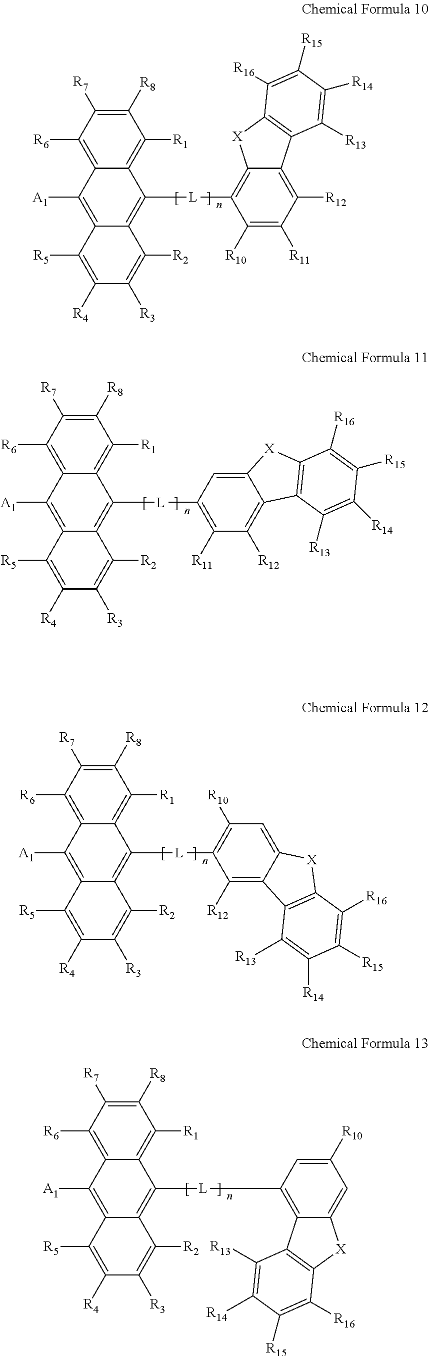
Wherein in Chemical Formulas 10 to 13,
R₁ to R₈ are the same as or different from each other, each independently being hydrogen or deuterium, provided that at least one of R₁ to R₈ is deuterium,
X is O, S or CRₐR_{b},
Rₐ and R_{b} are the same as or different from each other, each independently being a C₁ to C₄₀ alkyl group or a C₆ to C₆₀ aryl group, or Rₐ and R_{b} being bonded to each other to form a condensed ring, R₉ to R₁₆ are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group,
L is a single bond or is selected from: a C₆ to C₁₈ arylene group and a heteroarylene group having 5 to 18 nuclear atoms,
n is an integer in a range from 0 to 2,
A₁ is a substituent represented by the following Chemical Formula 2, 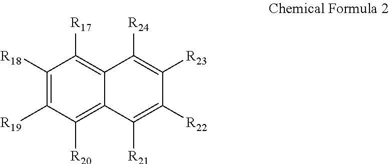
in Chemical Formula 2,
one of R₁₇ to R₂₄ is bonded to Chemical Formula 1, and another of R₁₇ to R₂₄ is bonded to an aryl group A₂ having at least one deuterium,
Chemical Formula 1 and the aryl group A₂ having at least one deuterium are bonded in an ortho position,
the others of R₁₇ to R₂₄ that are not bonded to Chemical Formula 1 and the aryl group having at least one deuterium are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group, and
the arylene group and the heteroarylene group of L and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the phosphine group, the phosphine oxide group, and the arylamine group of R₉ to R₁₄, Rₐ, R_{b}, and the others of R₁₇ to R₂₄ that are not bonded to Chemical Formula 1 and the aryl group having at least one deuterium are each independently substitutable with one or more kinds of substituents selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and when the substituents are plural in number, the substituents are the same as or different from each other.

33. The organic electroluminescent element of claim 30, wherein the compound of Chemical Formula 1 is represented by any one of Chemical Formulas 14 to 16 below: 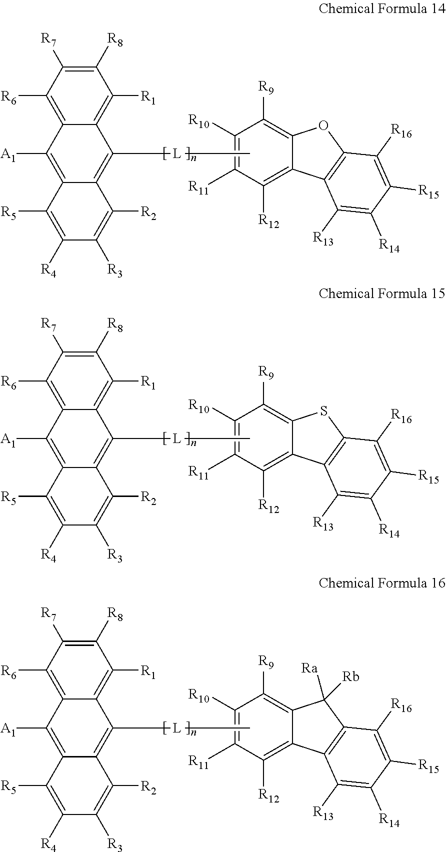
wherein in Chemical Formulas 14 to 16,
R₁ to R₈ are the same as or different from each other, each independently being hydrogen or deuterium, provided that at least one of R₁ to R₈ is deuterium,
X is O, S or CRₐR_{b},
Rₐ and R_{b} are the same as or different from each other, each independently being a C₁ to C₄₀ alkyl group or a C₆ to C₆₀ aryl group, or Rₐ and R_{b} being bonded to each other to form a condensed ring,
R₉ to R₁₆ are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group,
L is a single bond or is selected from: a C₆ to C₁₈ arylene group and a heteroarylene group having 5 to 18 nuclear atoms,
n is an integer in a range from 0 to 2,
A₁ is a substituent represented by the following Chemical Formula 2, 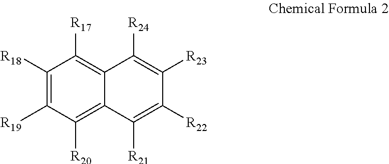
in Chemical Formula 2,
one of R₁₇ to R₂₄ is bonded to Chemical Formula 1, and another of R₁₇ to R₂₄ is bonded to an aryl group A₂ having at least one deuterium,
Chemical Formula 1 and the aryl group A₂ having at least one deuterium are bonded in an ortho position,
the others of R₁₇ to R₂₄ that are not bonded to Chemical Formula 1 and the aryl group having at least one deuterium are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group, and
the arylene group and the heteroarylene group of L and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the phosphine group, the phosphine oxide group, and the arylamine group of R₉ to R₁₄, Rₐ, R_{b}, and the others of R₁₇ to R₂₄ that are not bonded to Chemical Formula 1 and the aryl group having at least one deuterium are each independently substitutable with one or more kinds of substituents selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and when the substituents are plural in number, the substituents are the same as or different from each other.

34. The organic electroluminescent element of claim 30, wherein the compound of Chemical Formula 1 is represented by Chemical Formula 17 or Chemical Formula 18 below: 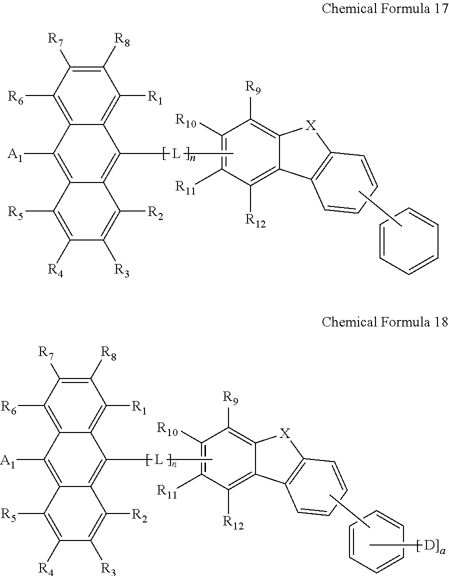
wherein in Chemical Formulas 17 to 18,
R₁ to R₈ are the same as or different from each other, each independently being hydrogen or deuterium, provided that at least one of R₁ to R₈ is deuterium,
X is O, S or CRₐR_{b},
Rₐ and R_{b} are the same as or different from each other, each independently being a C₁ to C₄₀ alkyl group or a C₆ to C₆₀ aryl group, or Rₐ and R_{b} being bonded to each other to form a condensed ring,
R₉ to R₁₂ are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group,
L is a single bond or is selected from: a C₆ to C₁₈ arylene group and a heteroarylene group having 5 to 18 nuclear atoms,
n is an integer in a range from 0 to 2,
A₁ is a substituent represented by the following Chemical Formula 2, 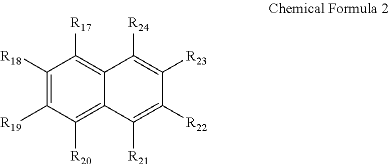
in Chemical Formula 2,
one of R₁₇ to R₂₄ is bonded to Chemical Formula 1, and another of R₁₇ to R₂₄ is bonded to an aryl group A₂ having at least one deuterium,
Chemical Formula 1 and the aryl group A₂ having at least one deuterium are bonded in an ortho position,
the others of R₁₇ to R₂₄ that are not bonded to Chemical Formula 1 and the aryl group having at least one deuterium are the same as or different from each other, each independently being selected from: hydrogen, deuterium, halogen, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, a C₆ to C₆₀ arylphosphine group, a C₆ to C₆₀ arylphosphine oxide group, and a C₆ to C₆₀ arylamine group, and
the arylene group and the heteroarylene group of L and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the phosphine group, the phosphine oxide group, and the arylamine group of R₉ to R₁₄, Rₐ, R_{b}, and the others of R₁₇ to R₂₄ that are not bonded to Chemical Formula 1 and the aryl group having at least one deuterium are each independently substitutable with one or more kinds of substituents selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and when the substituents are plural in number, the substituents are the same as or different from each other.

35. The organic electroluminescent element of claim 30, wherein the organic layer comprising the compound is selected from: a hole injection layer, a hole transport layer, a light emitting auxiliary layer, a light emitting layer, an electron transport layer, and an electron injection layer.

36. The organic electroluminescent element of claim 35, wherein the organic layer comprising the compound is a fluorescent light emitting layer.
